(19) United States
(12) Patent Application Publication
(10) Pub. No.: US 2024/0294489 A1
(43) Pub. Date: Sep. 5, 2024

(54) LOW MOLECULAR WEIGHT PROTEIN DEGRADERS AND THEIR APPLICATIONS

(71) Applicant: Captor Therapeutics S.A., Wroclaw (PL)

(72) Inventors: Sylvain COTTENS, Witterswil (CH); Niall DICKINSON, Skierniewice (PL); Katarzyna KACZANOWSKA, Wroclaw (PL); Krzysztofa ODRZYWÓL, Rozprza (PL); Roman PLUTA, Krakow (PL); Michal WALCZAK, Krakow (PL)

(21) Appl. No.: 18/040,383

(22) PCT Filed: Aug. 3, 2021
(86) PCT No.: PCT/EP2021/071694

§ 371 Date: Feb. 2, 2023

(30) Foreign Application Priority Data
Aug. 3, 2020 (WO) PCT/PL2020/000066

(51) Int. Cl. C07D 401/04; A61K 31/454; A61K 45/06; A61P 35/00; A61P 35/02; C07D 401/14; C07D 403/04; C07D 405/14

(52) U.S. Cl. CPC C07D 401/04; A61K 31/454; A61K 45/06; A61P 35/00; A61P 35/02; C07D 401/14; C07D 403/04; C07D 405/14

(57) ABSTRACT

The invention relates to compounds of formulae (Ia), (Ib), (Ic) and (II) and their use in methods of treating cancer. The compounds may be applied in combination with already existing cancer-fighting regimens to increase their effectiveness.

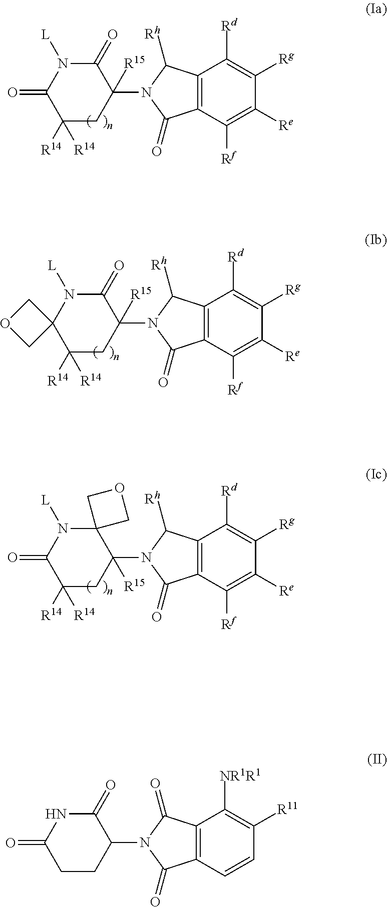

### CROSS-REFERENCE TO RELATED APPLICATIONS

This application is a U.S. National Stage Application under 35 U.S.C. § 371 of International Application No. PCT/EP2021/071694, filed Aug. 3, 2021, which claims priority to Patent Application No. PCT/PL2020/000066 (PL), filed Aug. 3, 2020, the contents of each of which are incorporated herein by reference in their entireties.

### FIELD OF THE INVENTION

The invention relates to compounds which modulate cellular concentrations of various disease-related proteins (for example, the transcription factor SALL4 and the translation termination factor GSPT1), and their applications.

### BACKGROUND

The Ubiquitin-Proteasome System (UPS) is responsible for the maintenance of healthy and well-balanced proteome. In the process of ubiquitination, ubiquitin units are covalently attached to the protein, forming a polyubiquitin chain, which marks the protein for degradation via the proteasome. Ubiquitination is central to the regulation of nearly all cellular processes and is also tightly regulated itself. Ubiquitin ligases such as cereblon (CRBN) facilitate ubiquitination of different proteins in vivo and contribute to precise regulation of the system. Upon recognition, the ubiquitin ligases mediate the attachment of ubiquitin moieties to the target protein, which label it for degradation by the proteasome.

The idea of selective target protein degradation (TPD) by modulation of UPS was first described in 1999 (US2002173049 A1 (PROTEINIX INC) 21 Nov. 2002). The implementation of this concept has been demonstrated for clinically approved thalidomide analogs, as binding of the thalidomide analogs to the CRL4^{CRBN} E3 ligase causes recruitment of selected target proteins, leading to their ubiquitination and subsequent proteasomal degradation. The recent scientific and clinical progress in TPD has been recently reviewed by Faust T B et al. Annu. Rev. Cancer Biol. 2021. 5:181-201.

### Cereblon Modulating Agents in the Treatment of Cancer

Cereblon (CRBN) is a protein which associates with DDB1 (damaged DNA binding protein 1), CUL4 (Cullin-4), and RBX1 (RING-Box Protein 1). Collectively, the proteins form a ubiquitin ligase complex, which belongs to Cullin RING Ligase (CRL) protein family and is referred to as CRL4^{CRBN}. Thalidomide, a drug approved for treatment of multiple myeloma in the late 1990s, binds to cereblon and modulates the substrate specificity of the CRL4^{CRBN} ubiquitin ligase complex. This mechanism underlies the pleiotropic effect of thalidomide on both immune cells and cancer cells (Lu G et al. Science. 2014 Jan. 17; 343(6168): 305-9).

The clinical applicability of cereblon modulating agents in numerous hematologic malignancies, such as multiple myeloma, myelodysplastic syndromes lymphomas and leukemia, has been demonstrated (Le Roy A et al. Front Immunol. 2018; 9: 977). The antitumor activity of CMAs is mediated by:
inhibition of cancer cell proliferation and induction of apoptosis,
disruption of trophic support from tumor stroma,
stimulation of immune cells, resulting in proliferation of T-cells, cytokine production and activation of NK (natural killer) cells.

Thalidomide's success in cancer therapy stimulated efforts towards development of analogues with higher potency and fewer detrimental side effects. As a result, various drug candidates were produced, including lenalidomide, pomalidomide, CC-220, CC-122, CC-885, and CC-90009. These compounds are collectively called Cereblon Modulating Agents (CMAs). For the discussion of these compounds, see—for example—U.S. Pat. No. 5,635,517(B2), WO2008039489 (A2), WO2017197055 (A1), WO2018237026 (A1), WO2017197051 (A1), U.S. Pat. No. 8,518,972 (B2), EP 2057143 (B1), WO2019014100 (A1), WO2004103274 (A2), and Surka Ch et al. Blood. 2021 Feb. 4; 137(5):661-677

Neosubstrate degradation profile of cereblon modulating agents mediates the phenotypic and clinical outcome in a context specific manner. For example, downregulation of lymphoid transcription factors IKZF1 (KAROS Family Zinc Finger 1) and IKZF3 (KAROS Family Zinc Finger 3) mediates clinical efficacy of lenalidomide and pomalidomide in multiple myeloma. Simultaneously, downregulation of IKZF1 and IKZ3 has been shown to contribute to the occurrence of side effects, that reduce the dose of the drug that can be administered to the patient suffering from myelodysplastic syndromes. Side effects occurring during the treatment with lenalidomide include neutropenia, leukopenia, thrombocytopenia, anemia, and hemorrhagic disorders (Stahl M et al. Cancer. 2017 May 15; 123(10):1703-1713). Thus, it is desired to advance the development of the cereblon modulating agents in order to achieve a desired substrate specificity of the CRL4^{CRBN} ubiquitin ligase complex to reach a desired efficacy and safety profile depending on the clinical context (Sievers Q L et al. Science. 2018 Nov. 2; 362 (6414).

### SALL4-Targeted Strategy in Eliminating Tumor Cells

Expression of Sal-like protein 4 (SALL4) transcription factor has been primarily detected in embryonic stem cells (ESCs) and adult germ cells and blood progenitor cells population, where it acts as a core controller regulating cell “stemness” in developmental events. However, SALL4 is re-activated and mis-regulated in various cancers, including: acute myeloid leukemia (AML), B-cell acute lymphocytic leukemia (B-ALL), germ cell tumors, breast cancer, hepatocellular carcinoma (HCC), lung cancer, glioma, and gastric cancer. Anomalous expression of SALL4 has been also detected in myelodysplastic syndrome (MDS) patients, its expression levels being correlated with disease progression. Additionally, SALL4 expression is correlated with worse survival and poor prognosis in hepatocellular carcinoma and with metastasis such as in endometrial cancer, colorectal carcinoma and esophageal squamous cell carcinoma (Yong K J et al. The New England Journal of Medicine, 2013, Forghanifard M M et. Al. Journal of Biomedical Science, 2013).

Downregulation of SALL4 causes increased apoptosis and cell cycle arrest (Gao C et al. Transfusion, 2013; Ma Y et al. Blood, 2006; Cao D et al. The American Journal of Surgical Pathology, 2009; Kobayashi D et al. International Journal of Oncology, 2011, Oikawa T et al. Hepatology, 2013, Morita S et al. The American Journal of Surgical Pathology, 2013, Zhang L et al.: Journal of Neuro-Oncology, 2015; Wang F et al. Journal of Hematology Oncology, 2013; Zhang L et al.: Oncogene, 2013). SALL4-derived peptide blocking its protein-protein interaction with the nucleosome remodeling and histone deacetylation (NuRD) complex results in notable leukemic cell death, but causes no cytotoxic effects on normal CD34+HSCs/HPCs (Gao C et al. Blood, 2013).

Studies show that up-regulating SALL4 in cancer cells can promote their proliferative and invasive abilities, as well as tumor chemo-resistance and down-regulation of SALL4 suppresses the growth of cancer cells. Additional approach to find more effective and efficient methods of cancer cell elimination is the combination of already existing practices. Here, the downregulation of SALL4 is a way to sensitize tumor cells to standard of care cancer therapy, such as surgery, chemotherapy, hormonal therapy, radiation treatment and/or biological therapy, and immunotherapy.

### GSPT1-Targeted Strategy in Eliminating Tumor Cells

GSPT1 is a translation termination factor downregulation of which may activate an integrated stress response leading to cancer cell death. It has been demonstrated that GSPT1 depletion plays a significant functional role in the anti-AML activity of CC-90009, which is now under the clinical development. GSPT1 degradation activates the GCN1/GCN2/eIF2α/ATF4 axis of the integrated stress response, and subsequent induction of acute apoptosis in AML (Surka Ch et al. Blood. 2021 Feb. 4; 137(5):661-677).

### SUMMARY OF INVENTION

The invention provides compounds which can modulate levels of target disease-related proteins (for example, SALL4 and GSPT1) in vitro and in vivo. The compounds of the invention exhibit preferential degradation of the target proteins resulting in a unique phenotypic profile.

The invention further provides a method for treating cancer, which method comprises administering the patient with a pharmaceutical composition comprising a compound of the present invention.

The invention relates to the developing of a drug candidate that inhibits the development of cancer and/or increases the effectiveness of currently available therapies. The small molecule drug efficacy relies on the induced degradation of the preferentially-targeted protein. An example of a protein which is preferentially targeted by the compounds of the present invention is SALL4, which plays an important role in the process of carcinogenesis and its progression. Another protein which is preferentially targeted by the compounds of the invention is GSPT1.

The invention provides a way to modulate the expression level of the therapeutic proteins, e.g., to increase efficacy and/or decrease side effects. The invention provides compounds which cause preferential degradation of specific targets (e.g. SALL4), thus provides a new mechanism of therapeutic activity for proteins that would not otherwise be susceptible to the action of small molecule compounds.

The compounds of the present invention potently inhibit growth of several cancer types: hepatocellular carcinoma (HEP3B, SNU-398), neuroblastoma (Kelly), leukemia (KG-1, KG-1a, UOC-M1, MOLT-3, MOLT-4, MOLM-13, MOLM-1, MOLM-6) prostate cancer (22Rv1), multiple myeloma (MOLP-2). Simultaneously, the compounds of the present invention do not exhibit activity towards H929 and various other cell lines (Table 10 and 12) which makes them unique in comparison to the known compounds CC-90009, Lenalidomide, Pomalidomide, CC-122, and CC-220. This surprising effect makes the compounds clinically attractive due to their enhanced selectivity that will likely correspond to a therapeutic window in particular cancer types, such as HCC.

The developed SALL4 degrading drug candidates can be applied to treatment of novel cancer types, where known IMiDs are not applicable.

The use of the compounds of the invention may eliminate side effects that occur in patients taking lenalidomide. Since these effects of lenalidomide result from degradation of IKZF1/IKZF3, they may be eliminated by using the compounds of the present invention.

To minimize occurrence of the potential adverse side effects resulting from the degradation of IKZF1 or IKZF3, drug candidates which target SALL4 preferentially are presented, with no or minor activity against indicated proteins as compared to current IMiD drugs.

The compounds of the present invention, which have high preference for SALL4 protein degradation, and induce protein degradation potently and rapidly, will significantly improve the prognosis of patients with cancers.

In a first aspect, the present invention provides a compound of formula (Ia), (Ib) or (Ic):

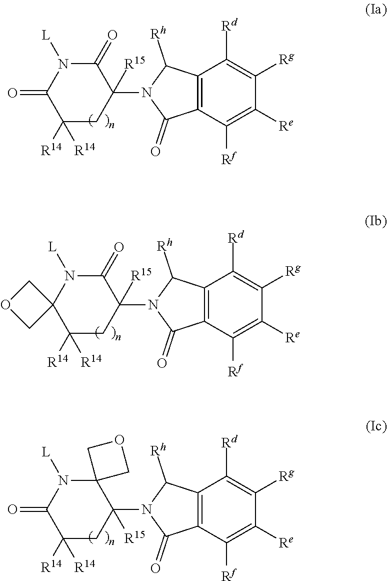

or a pharmaceutically acceptable salt, ester, optically active isomer, racemate, solvate, amino acid conjugate, or prodrug thereof, wherein
L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″ or P(O)(OR″)(OR″);
each R″ is independently selected from hydrogen, alkyl, cycloalkyl, alkenyl, cycloalkenyl, aryl, heteroaryl, or benzyl;
each R¹⁴ is independently selected from deuterium and hydrogen;
R¹⁵ is selected from hydrogen, deuterium and C₁-C₄ alkyl;
R^{g} is CR^{a}R^{b}R^{c},
R^{h} is selected from H and C₁-C₄ alkyl;
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
each of R^{d}, R^{e} and R^{f} is independently selected from H, deuterium, X, C₁-C₄ alkyl, and NH₂;
n is 0, 1 or 2;
X is selected from F, Cl, Br and I;
R¹ is selected from H and C₁-C₃ alkyl,
R² is selected from H, C₁-C₃ alkyl, —COR₃, and —COOR₃,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is selected from:
   unsubstituted C₁-C₄ alkyl;
   C₁-C₁₀ alkyl substituted with one or more R⁴, wherein each R⁴ is independently selected from NH₂, NHC(NH)NH₂, NHCOR⁵, NHCOOR⁵, —OH, OR⁵, OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, —OH, —CH₂—OH, —OCO(C₁-C₄ alkyl) and CH₂OCO(C₁-C₄ alkyl); and wherein R⁴ is not X;
   C₂-C₁₀ alkyl substituted with a halophenyl group;
   6-membered aryl substituted with one or more substituents independently selected from CH₂—OH or CH₂OCO(C₁-C₄ alkyl);
   unsubstituted 5- or 6-membered heterocyclyl
R⁵ is unsubstituted C₁-C₆ alkyl; or C₁-C₆ alkyl substituted with one or more substituents independently selected from 5-membered heterocyclyl, 6-membered heterocyclyl, 5-membered aryl, 6-membered aryl, 5-membered heteroaryl and 6-membered heteroaryl; and
R⁶ is unsubstituted cyclopentyl or cyclohexyl; or cyclopentyl or cyclohexyl substituted with one or more NH₂;
wherein, in formula (Ia):
when R^{a}, R^{b}, R¹ and R² are each H, then n is 0 or 1;
when R^{a}, R^{b}, R^{d}, R^{e}, R^{f}, R^{h}, R¹, R², R¹⁴ and L are each H, and R¹⁵ is H or C₁-C₄ alkyl, then n is 0;
when R^{a}, R^{b} and R¹ are each H and R² is —COR³, then n is 0 or 1; and
when R^{a}, R^{b}, R^{d}, R^{e}, R^{f} and R¹ are each H and R³ is unsubstituted C₁-C₄ alkyl, then n is 0.

In some embodiments, R³ is selected from:
unsubstituted C₁-C₄ alkyl;
C₁-C₁₀ alkyl substituted with one or more R⁴, wherein each R⁴ is independently selected from NH₂, NHC(NH)NH₂, NHCOR⁵, NHCOOR⁵, —OH, OR⁵, OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, OH, —CH—OH, —OCO(C₁-C₄ alkyl) and CH₂OCO(C₁-C₄ alkyl); and wherein R⁴ is not X;
6-membered aryl substituted with one or more substituents independently selected from CH₂—OH or CH₂OCO(C₁-C₄ alkyl); and
unsubstituted 5- or 6-membered heterocyclyl.

In some embodiments, R³ is selected from:
unsubstituted C₁-C₄ alkyl;
C₁-C₁₀ alkyl substituted with one or more R⁴, wherein each R⁴ is independently selected from NH₂, NHC(NH)NH₂, NHCOR⁵, NHCOOR⁵, —OH, OR⁵, OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, —OH, —CH₂—OH, —OCO(C₁-C₄ alkyl) and CH₂OCO(C₁-C₄ alkyl); wherein R⁴ is not X, and wherein when the C₁-C₁₀ alkyl is substituted with indole, the C₁-C₁₀ alkyl is further substituted with at least one additional R⁴;
6-membered aryl substituted with one or more substituents independently selected from CH₂—OH or CH₂OCO(C₁-C₄ alkyl); and
unsubstituted 5- or 6-membered heterocyclyl.

In a second aspect, the present invention provides a compound of formula (II):

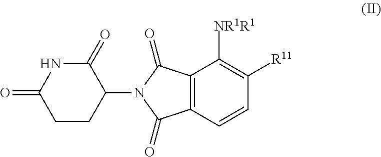

or a pharmaceutically acceptable salt, ester, optically active isomer, racemate, solvate, amino acid conjugate, or prodrug thereof,
wherein:
each R¹ is independently selected from H and C₁-C₄ alkyl;
R¹¹ is OH or OR^{5a}; and
R^{5a} is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

In some embodiments of any of the above aspects, R¹¹ is OH.

In some embodiments of any of the above aspects, NR¹R¹ is NH₂. In some embodiments of any of the above aspects, R^{h} is H. In other embodiments, R^{h} is methyl.

In some embodiments of any of the above aspects, R^{a} and R^{b} are each H. In other embodiments R^{a} and R^{b} are each deuterium. In other embodiments, R^{a} is H and R^{b} is methyl.

In some embodiments of any of the above aspects, R^{c} is selected from NHR², and OH.

In some embodiments of any of the above aspects the compound is selected from:

| Compound | |
|---|---|
| ID | Structure |
| 2 | 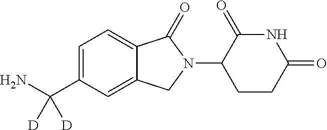 [SMILES: [2H]C([2H])(N)c1ccc2c(c1)CN(C1CCC(=O)NC1=O)C2=O] |
| 3 | 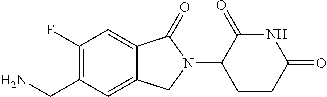 [SMILES: NCc1cc2c(cc1F)C(=O)N(C1CCC(=O)NC1=O)C2] |
| 4 | 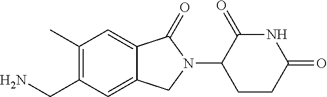 [SMILES: Cc1cc2c(cc1CN)CN(C1CCC(=O)NC1=O)C2=O] |
| 5 | 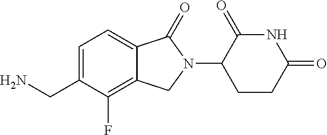 [SMILES: NCc1ccc2c(c1F)CN(C1CCC(=O)NC1=O)C2=O] |
| 6 | 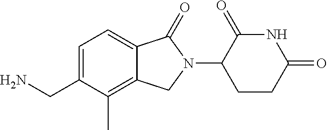 [SMILES: Cc1c(CN)ccc2c1CN(C1CCC(=O)NC1=O)C2=O] |
| 7 | 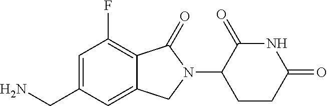 [SMILES: NCc1cc(F)c2c(c1)CN(C1CCC(=O)NC1=O)C2=O] |
| 8 | 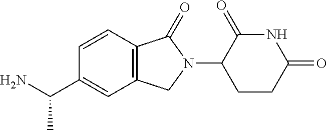 [SMILES: C[C@H](N)c1ccc2c(c1)CN(C1CCC(=O)NC1=O)C2=O] |
| 9 | 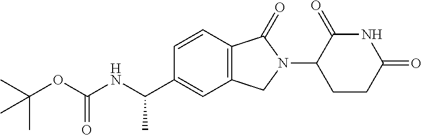 [SMILES: C[C@H](NC(=O)OC(C)(C)C)c1ccc2c(c1)CN(C1CCC(=O)NC1=O)C2=O] |
| 10 | 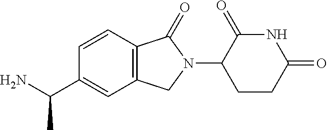 [SMILES: C[C@@H](N)c1ccc2c(c1)CN(C1CCC(=O)NC1=O)C2=O] |
| 11 | 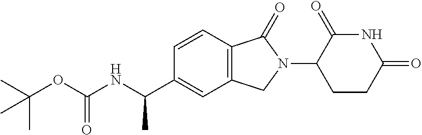 [SMILES: C[C@@H](NC(=O)OC(C)(C)C)c1ccc2c(c1)CN(C1CCC(=O)NC1=O)C2=O] |
| 13 | 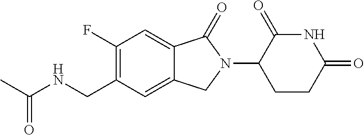 [SMILES: CC(=O)NCc1cc2c(cc1F)C(=O)N(C1CCC(=O)NC1=O)C2] |
| 14 | 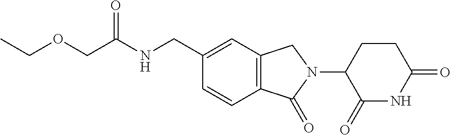 [SMILES: CCOCC(=O)NCc1ccc2c(c1)CN(C1CCC(=O)NC1=O)C2=O] |
| 16 | 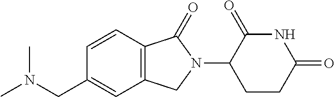 [SMILES: CN(C)Cc1ccc2c(c1)CN(C1CCC(=O)NC1=O)C2=O] |
| 17 | 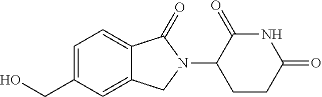 [SMILES: O=C1CCC(N2Cc3cc(CO)ccc3C2=O)C(=O)N1] |
| 18 | 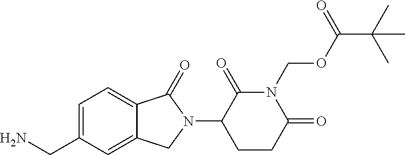 [SMILES: CC(C)(C)C(=O)OCN1C(=O)CCC(N2Cc3cc(CN)ccc3C2=O)C1=O] |
| 19 | 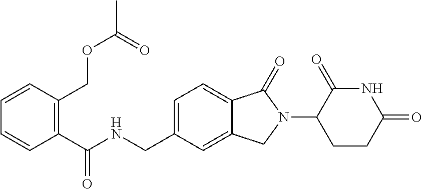 [SMILES: CC(=O)OCc1ccccc1C(=O)NCc1ccc2c(c1)CN(C1CCC(=O)NC1=O)C2=O] |
| 20 | 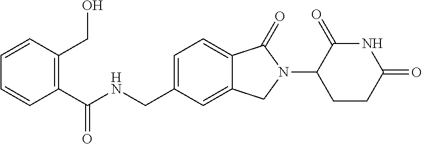 [SMILES: O=C1CCC(N2Cc3cc(CNC(=O)c4ccccc4CO)ccc3C2=O)C(=O)N1] |
| 21 | 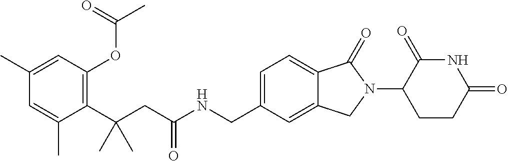 [SMILES: CC(=O)Oc1cc(C)cc(C)c1C(C)(C)CC(=O)NCc1ccc2c(c1)CN(C1CCC(=O)NC1=O)C2=O] |
| 22 | 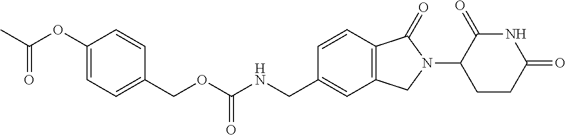 [SMILES: CC(=O)Oc1ccc(COC(=O)NCc2ccc3c(c2)CN(C2CCC(=O)NC2=O)C3=O)cc1] |
| 23 | 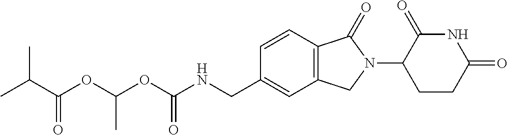 [SMILES: CC(OC(=O)NCc1ccc2c(c1)CN(C1CCC(=O)NC1=O)C2=O)OC(=O)C(C)C] |
| 24 | 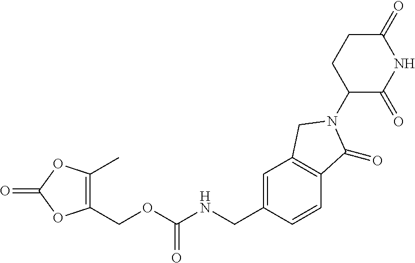 [SMILES: Cc1oc(=O)oc1COC(=O)NCc1ccc2c(c1)CN(C1CCC(=O)NC1=O)C2=O] |
| 25 | 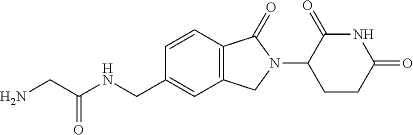 [SMILES: NCC(=O)NCc1ccc2c(c1)CN(C1CCC(=O)NC1=O)C2=O] |
| 26 | 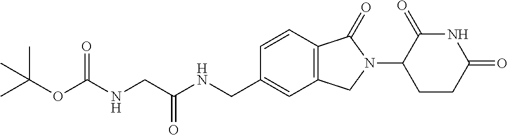 [SMILES: CC(C)(C)OC(=O)NCC(=O)NCc1ccc2c(c1)CN(C1CCC(=O)NC1=O)C2=O] |
| 27 | 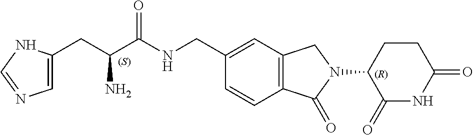 |
| 28 | 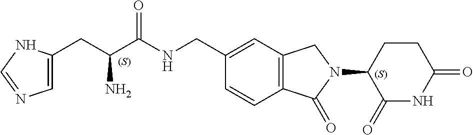 |
| 29 | 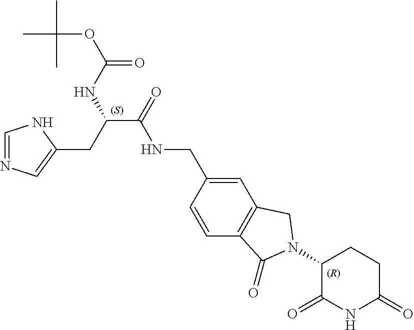 |
| 30 | 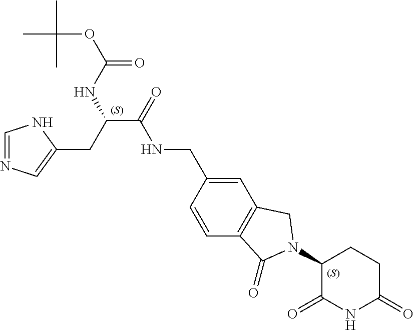 |
| 31 | 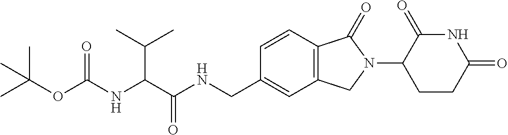 [SMILES: CC(C)C(NC(=O)OC(C)(C)C)C(=O)NCc1ccc2c(c1)CN(C1CCC(=O)NC1=O)C2=O] |
| 32 | 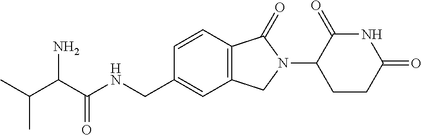 [SMILES: CC(C)C(N)C(=O)NCc1ccc2c(c1)CN(C1CCC(=O)NC1=O)C2=O] |
| 33 | 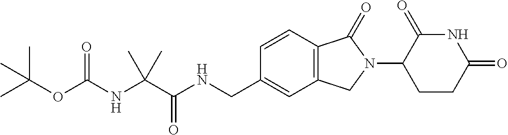 [SMILES: CC(C)(C)OC(=O)NC(C)(C)C(=O)NCc1ccc2c(c1)CN(C1CCC(=O)NC1=O)C2=O] |
| 34 | 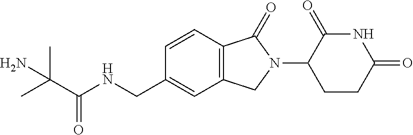 [SMILES: CC(C)(N)C(=O)NCc1ccc2c(c1)CN(C1CCC(=O)NC1=O)C2=O] |
| 35 | 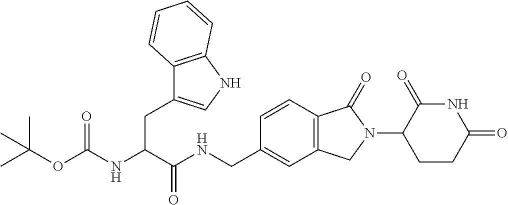 [SMILES: CC(C)(C)OC(=O)NC(Cc1c[nH]c2ccccc12)C(=O)NCc1ccc2c(c1)CN(C1CCC(=O)NC1=O)C2=O] |
| 37 | 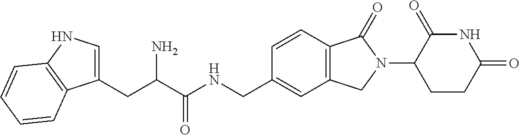 [SMILES: NC(Cc1c[nH]c2ccccc12)C(=O)NCc1ccc2c(c1)CN(C1CCC(=O)NC1=O)C2=O] |
| 38 | 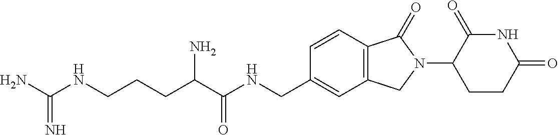 [SMILES: N=C(N)NCCCC(N)C(=O)NCc1ccc2c(c1)CN(C1CCC(=O)NC1=O)C2=O] |
| 39 | 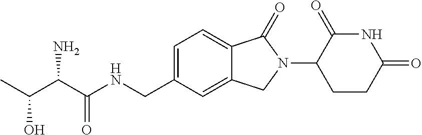 [SMILES: C[C@@H](O)[C@H](N)C(=O)NCc1ccc2c(c1)CN(C1CCC(=O)NC1=O)C2=O] |
| 40 | 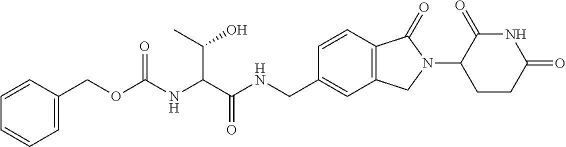 [SMILES: C[C@H](O)C(NC(=O)OCc1ccccc1)C(=O)NCc1ccc2c(c1)CN(C1CCC(=O)NC1=O)C2=O] |
| 41 | 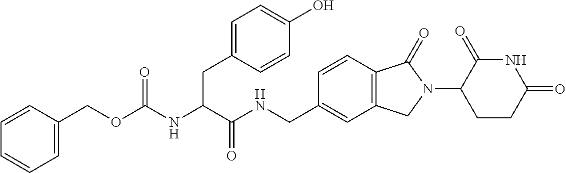 [SMILES: O=C1CCC(N2Cc3cc(CNC(=O)C(Cc4ccc(O)cc4)NC(=O)OCc4ccccc4)ccc3C2=O)C(=O)N1] |
| 43 | 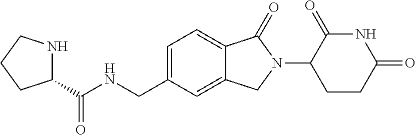 [SMILES: O=C1CCC(N2Cc3cc(CNC(=O)[C@@H]4CCCN4)ccc3C2=O)C(=O)N1] |
| 50 | 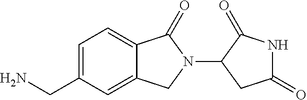 [SMILES: NCc1ccc2c(c1)CN(C1CC(=O)NC1=O)C2=O] |
| 52 | 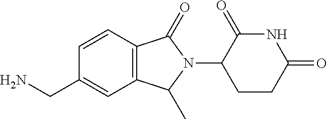 [SMILES: CC1c2cc(CN)ccc2C(=O)N1C1CCC(=O)NC1=O] |

and pharmaceutical acceptable salts, esters, optically active isomers, racemates, solvates, amino acid conjugates, or prodrugs thereof.

In some embodiments of any of the above aspects, R^{c} is NHR².

In some embodiments of any of the above aspects, R² is selected from H, —COR³, and —COOR³

In some embodiments of any of the above aspects, R³ is C₁-C₁₀ alkyl substituted with one or more R⁴. In some embodiments, each R⁴ is independently selected from NH₂, OCOR⁵, substituted or unsubstituted dioxolyl, indole, and 6-membered aryl substituted with one or more —OCO(C₁-C₄ alkyl); wherein R⁴ is not X.

In some embodiments of any of the above aspects, the compound is selected from compounds 51, 2, 22, 3, 24, 6, 23, 52, and 37:

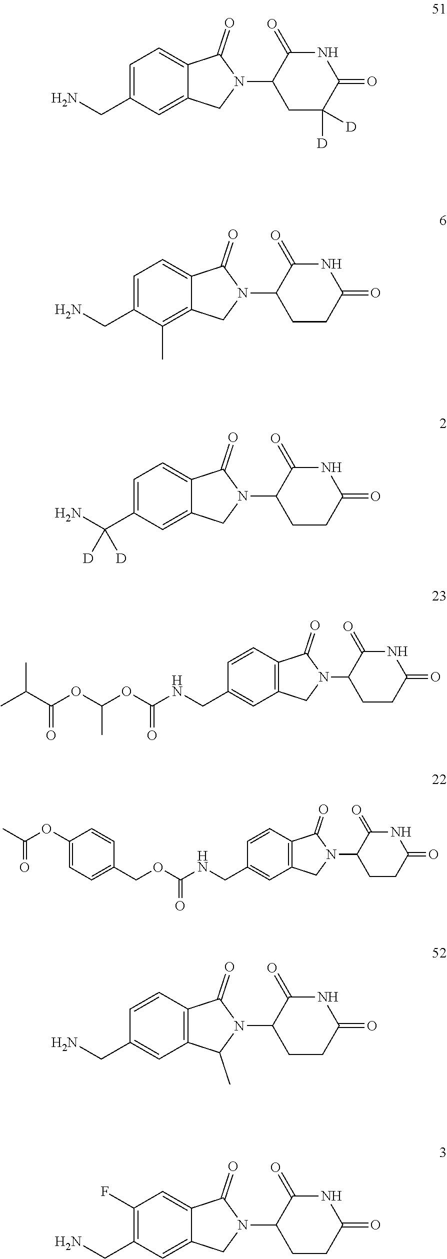

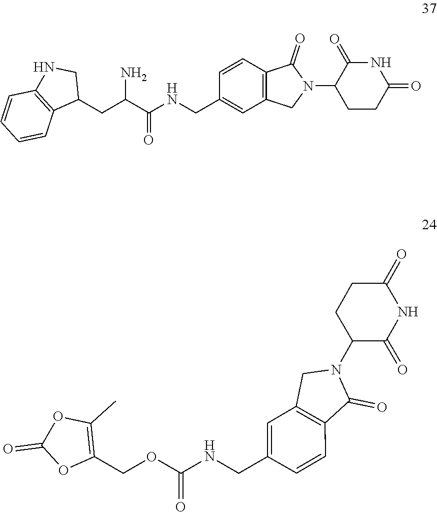 [SMILES: NC(CC1CNc2ccccc21)C(=O)NCc1ccc2c(c1)CN(C1CCC(=O)NC1=O)C2=O ; Cc1oc(=O)oc1COC(=O)NCc1ccc2c(c1)CN(C1CCC(=O)NC1=O)C2=O]

and pharmaceutically acceptable salts, esters, optically active isomers, racemates, solvates, amino acid conjugates, or prodrugs thereof.

In a third aspect, the present invention provides a pharmaceutical composition comprising a compound of any of the embodiments above.

In a fourth aspect, the present invention provides a compound for use in a method of treating cancer, the method comprising administering the compound to a subject in need thereof, wherein the compound is:
(i) a compound of formula (Ia), (Ib) or (Ic):

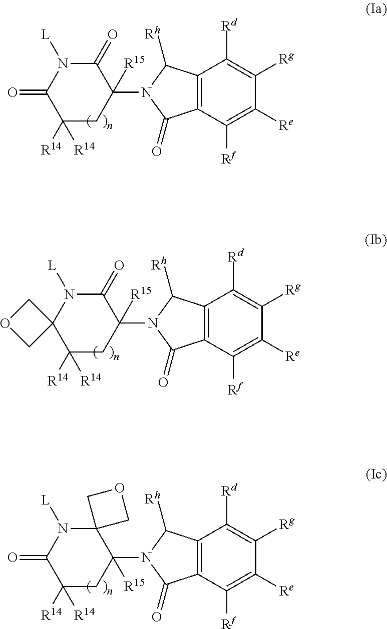

or a pharmaceutically acceptable salt, ester, optically active isomer, racemate, solvate, amino acid conjugate, or prodrug thereof, wherein
L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″ or P(O)(OR″)(OR″);
each R″ is independently selected from hydrogen, alkyl, cycloalkyl, alkenyl, cycloalkenyl, aryl, heteroaryl, or benzyl;
each R¹⁴ is independently selected from deuterium and hydrogen;
R¹⁵ is selected from hydrogen, deuterium and C₁-C₄ alkyl;
R^{g} is selected from —COOH and CR^{a}R^{b}R^{c},
R^{h} is selected from H and C₁-C₄ alkyl;
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
each of R^{d}, R^{e} and R^{f} is independently selected from H, deuterium, X, C₁-C₄ alkyl, and NH₂;
n is 0, 1 or 2;
X is selected from F, Cl, Br and I;
R¹ is selected from H and C₁-C₄ alkyl,
R² is selected from H, C₁-C₄ alkyl, —COR³, and —COOR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is selected from:
   unsubstituted C₁-C₄ alkyl;
   C₁-C₁₀ alkyl substituted with one or more R⁴, wherein each R⁴ is independently selected from NH₂, NHC(NH)NH₂, NHCOR⁵, NHCOOR⁵, —OH, OR⁵, OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, OH, —CH—OH, —OCO(C₁-C₄ alkyl) and CH₂OCO(C₁-C₄ alkyl); and wherein R⁴ is not X;
   C₂-C₁₀ alkyl substituted with a halophenyl group;
   6-membered aryl substituted with one or more substituents independently selected from CH₂—OH or CH₂OCO(C₁-C₄ alkyl); and
   unsubstituted 5- or 6-membered heterocyclyl
R⁵ is unsubstituted C₁-C₆ alkyl; or C₁-C₆ alkyl substituted with one or more substituents independently selected from 5-membered heterocyclyl, 6-membered heterocyclyl, 5-membered aryl, 6-membered aryl, 5-membered heteroaryl and 6-membered heteroaryl; and
R⁶ is unsubstituted cyclopentyl or cyclohexyl; or cyclopentyl or cyclohexyl substituted with one or more NH₂;
wherein when R^{a}, R^{b} and R¹ are each H and R² is H or —COR³, then n is 0 or 1;
or
(ii) a compound of formula (II):

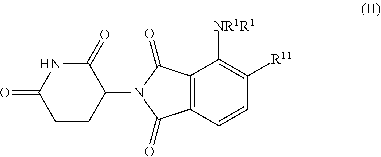

or a pharmaceutically acceptable salt, ester, optically active isomer, racemate, solvate, amino acid conjugate, or prodrug thereof,
wherein:
each R¹ is independently selected from H and C₁-C₄ alkyl;
R¹¹ is OH or OR^{5a}; and
R^{5a} is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

In a fifth aspect, the present invention provides a pharmaceutical composition for use in a method of treating cancer, the method comprising administering the pharmaceutical composition to a subject in need thereof, wherein the pharmaceutical composition comprises:
(i) a compound of formula (Ia), (Ib) or (Ic):

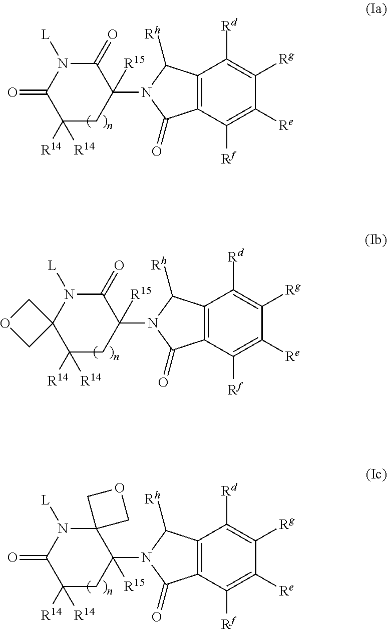

or a pharmaceutically acceptable salt, ester, optically active isomer, racemate, solvate, amino acid conjugate, or prodrug thereof,

wherein
L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″ or P(O)(OR″)(OR″);
each R″ is independently selected from hydrogen, alkyl, cycloalkyl, alkenyl, cycloalkenyl, aryl, heteroaryl, or benzyl;
each R¹⁴ is independently selected from deuterium and hydrogen;
R¹⁵ is selected from hydrogen, deuterium and C₁-C₄ alkyl;
R⁹ is selected from —COOH and CR^{a}R^{b}R^{c},
R^{h} is selected from H and C₁-C₄ alkyl;
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
each of R^{d}, R^{e} and R^{f} is independently selected from H, deuterium, X, C₁-C₄ alkyl, and NH₂;
n is 0, 1 or 2;
X is selected from F, Cl, Br and I;
R¹ is selected from H and C₁-C₄ alkyl,
R² is selected from H, C₁-C₄ alkyl, —COR³, and —COOR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is selected from:
   unsubstituted C₁-C₄ alkyl;
   C₁-C₁₀ alkyl substituted with one or more R⁴, wherein each R⁴ is independently selected from NH₂, NHC(NH)NH₂, NHCOR⁵, NHCOOR⁵, OH, OR⁵, OCOR, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, —OH, —CH₂—OH, —OCO(C₁-C₄ alkyl) and CH₂OCO(C₁-C₄ alkyl); and wherein R⁴ is not X;
   C₂-C₁₀ alkyl substituted with a halophenyl group;
   6-membered aryl substituted with one or more substituents independently selected from CH₂—OH or CH₂OCO(C₁-C₄ alkyl); and
   unsubstituted 5- or 6-membered heterocyclyl;
R⁵ is unsubstituted C₁-C₆ alkyl; or C₁-C₆ alkyl substituted with one or more substituents independently selected from 5-membered heterocyclyl, 6-membered heterocyclyl, 5-membered aryl, 6-membered aryl, 5-membered heteroaryl and 6-membered heteroaryl; and
R⁶ is unsubstituted cyclopentyl or cyclohexyl; or cyclopentyl or cyclohexyl substituted with one or more NH₂;
wherein when R^{a}, R^{b} and R¹ are each H and R² is H or —COR³, then n is 0 or 1;
or
(ii) a compound of formula (II):

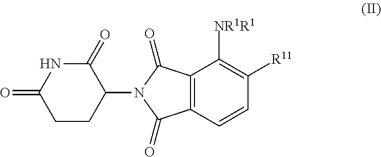

or a pharmaceutically acceptable salt, ester, optically active isomer, racemate, solvate, amino acid conjugate, or prodrug thereof,
wherein:
each R¹ is independently selected from H and C₁-C₄ alkyl;
R¹¹ is OH or OR^{5a}; and
R^{5a} is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

In a sixth aspect, the present invention provides a method of treating cancer comprising administering to a subject in need thereof a compound or pharmaceutical composition as described in any of the fourth and fifth aspects.

In some embodiments of any of the fourth to sixth aspects, R³ is selected from:
unsubstituted C₁-C₄ alkyl;
C₁-C₁₀ alkyl substituted with one or more R⁴, wherein each R⁴ is independently selected from NH₂, NHC(NH)NH₂, NHCOR⁵, NHCOOR⁵, —OH, OR⁵, OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, OH, —CH—OH, —OCO(C₁-C₄ alkyl) and CH₂OCO(C₁-C₄ alkyl); and wherein R⁴ is not X;
6-membered aryl substituted with one or more substituents independently selected from CH₂—OH or CH₂OCO(C₁-C₄ alkyl); and
unsubstituted 5- or 6-membered heterocyclyl.

In some embodiments of any of the fourth to sixth aspects, R³ is selected from:
unsubstituted C₁-C₄ alkyl;
C₁-C₁₀ alkyl substituted with one or more R⁴, wherein each R⁴ is independently selected from NH₂, NHC(NH)NH₂, NHCOR⁵, NHCOOR⁵, —OH, OR⁵, OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, —OH, —CH₂—OH, —OCO(C₁-C₄ alkyl) and CH₂OCO(C₁-C₄ alkyl); wherein R⁴ is not X, and wherein when the C₁-C₁₀ alkyl is substituted with indole, the C₁-C₁₀ alkyl is further substituted with at least one additional R⁴;
6-membered aryl substituted with one or more substituents independently selected from CH₂—OH or CH₂OCO(C₁-C₄ alkyl); and
unsubstituted 5- or 6-membered heterocyclyl.

In some embodiments of any of the fourth to sixth aspects, R¹¹ is OH.

In some embodiments of any of the fourth to sixth aspects, NR¹R¹ is NH₂.

In some embodiments of any of the fourth to sixth aspects, R^{h} is H. in other embodiments, R^{h} is methyl.

In some embodiments of any of the fourth to sixth aspects, R^{a} and R^{b} are each H. in other embodiments, R^{a} and R^{b} are each deuterium. In other embodiments, R^{a} is H and R^{b} is methyl.

In some embodiments of any of the fourth to sixth aspects, R^{c} is selected from NHR² and OH.

In some embodiments of any of the fourth to sixth aspects, the compound is selected from the compounds in Table 1:

TABLE 1

| Compound | |
|---|---|
| ID | Structure |
| 1 | 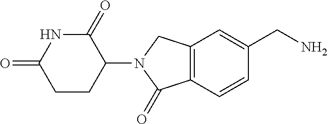 [SMILES: NCc1ccc2c(c1)CN(C1CCC(=O)NC1=O)C2=O] |
| 2 | 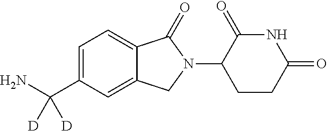 [SMILES: [2H]C([2H])(N)c1ccc2c(c1)CN(C1CCC(=O)NC1=O)C2=O] |
| 3 | 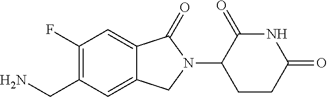 [SMILES: NCc1cc2c(cc1F)C(=O)N(C1CCC(=O)NC1=O)C2] |
| 4 | 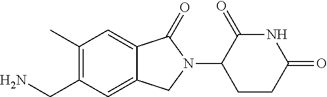 [SMILES: Cc1cc2c(cc1CN)CN(C1CCC(=O)NC1=O)C2=O] |
| 5 | 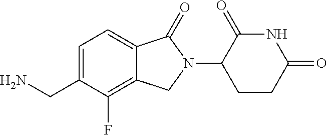 [SMILES: NCc1ccc2c(c1F)CN(C1CCC(=O)NC1=O)C2=O] |
| 6 | 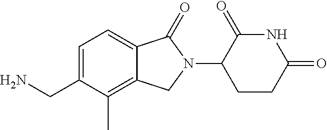 [SMILES: Cc1c(CN)ccc2c1CN(C1CCC(=O)NC1=O)C2=O] |
| 7 | 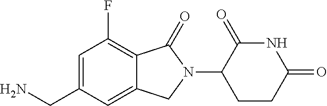 [SMILES: NCc1cc(F)c2c(c1)CN(C1CCC(=O)NC1=O)C2=O] |
| 8 | 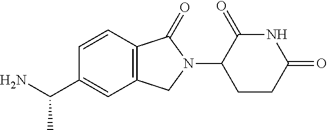 [SMILES: C[C@H](N)c1ccc2c(c1)CN(C1CCC(=O)NC1=O)C2=O] |
| 9 | 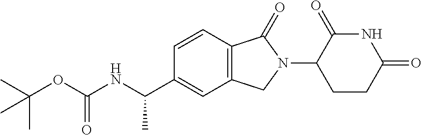 [SMILES: C[C@H](NC(=O)OC(C)(C)C)c1ccc2c(c1)CN(C1CCC(=O)NC1=O)C2=O] |
| 10 | 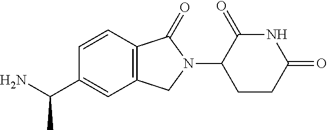 [SMILES: C[C@@H](N)c1ccc2c(c1)CN(C1CCC(=O)NC1=O)C2=O] |
| 11 | 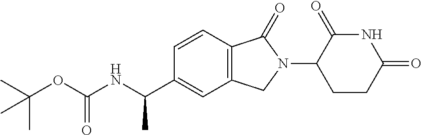 [SMILES: C[C@@H](NC(=O)OC(C)(C)C)c1ccc2c(c1)CN(C1CCC(=O)NC1=O)C2=O] |
| 12 | 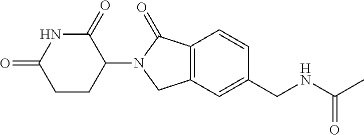 [SMILES: CC(=O)NCc1ccc2c(c1)CN(C1CCC(=O)NC1=O)C2=O] |
| 13 | 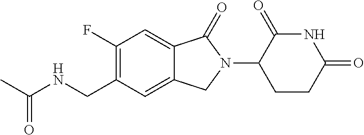 [SMILES: CC(=O)NCc1cc2c(cc1F)C(=O)N(C1CCC(=O)NC1=O)C2] |
| 14 | 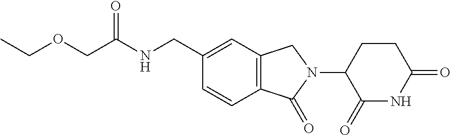 [SMILES: CCOCC(=O)NCc1ccc2c(c1)CN(C1CCC(=O)NC1=O)C2=O] |
| 15 | 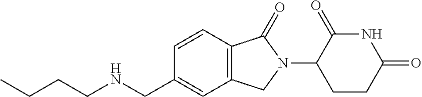 [SMILES: CCCCNCc1ccc2c(c1)CN(C1CCC(=O)NC1=O)C2=O] |
| 16 | 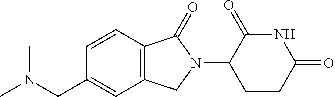 [SMILES: CN(C)Cc1ccc2c(c1)CN(C1CCC(=O)NC1=O)C2=O] |
| 17 | 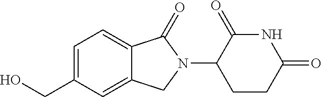 [SMILES: O=C1CCC(N2Cc3cc(CO)ccc3C2=O)C(=O)N1] |
| 18 | 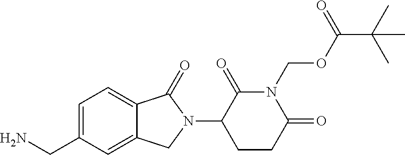 [SMILES: CC(C)(C)C(=O)OCN1C(=O)CCC(N2Cc3cc(CN)ccc3C2=O)C1=O] |
| 19 | 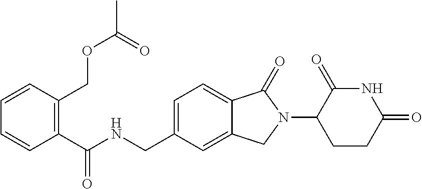 [SMILES: CC(=O)OCc1ccccc1C(=O)NCc1ccc2c(c1)CN(C1CCC(=O)NC1=O)C2=O] |
| 20 | 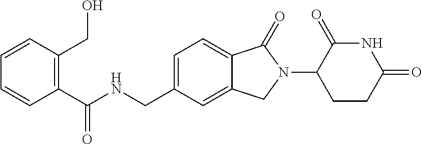 [SMILES: O=C1CCC(N2Cc3cc(CNC(=O)c4ccccc4CO)ccc3C2=O)C(=O)N1] |
| 21 | 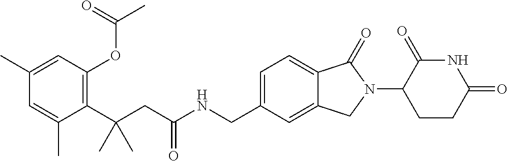 [SMILES: CC(=O)Oc1cc(C)cc(C)c1C(C)(C)CC(=O)NCc1ccc2c(c1)CN(C1CCC(=O)NC1=O)C2=O] |
| 22 | 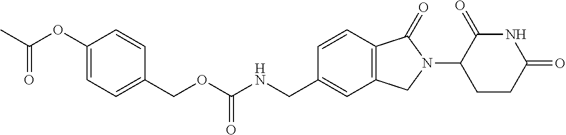 [SMILES: CC(=O)Oc1ccc(COC(=O)NCc2ccc3c(c2)CN(C2CCC(=O)NC2=O)C3=O)cc1] |
| 23 | 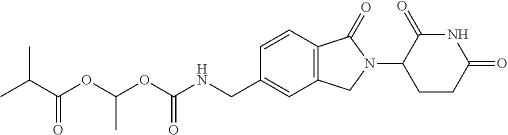 [SMILES: CC(OC(=O)NCc1ccc2c(c1)CN(C1CCC(=O)NC1=O)C2=O)OC(=O)C(C)C] |
| 24 | 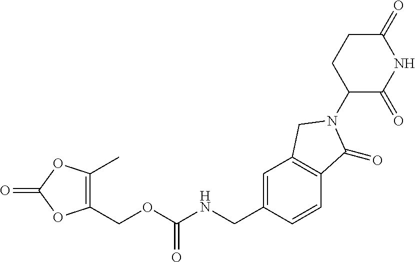 [SMILES: Cc1oc(=O)oc1COC(=O)NCc1ccc2c(c1)CN(C1CCC(=O)NC1=O)C2=O] |
| 25 | 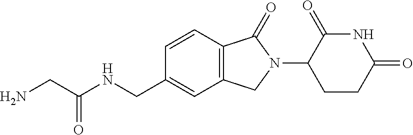 [SMILES: NCC(=O)NCc1ccc2c(c1)CN(C1CCC(=O)NC1=O)C2=O] |
| 26 | 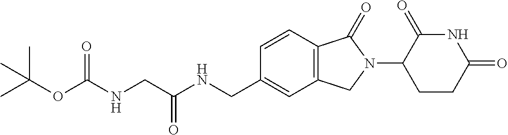 [SMILES: CC(C)(C)OC(=O)NCC(=O)NCc1ccc2c(c1)CN(C1CCC(=O)NC1=O)C2=O] |
| 27 | 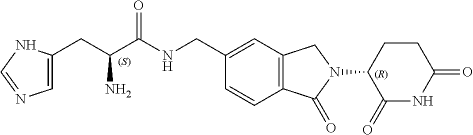 |
| 28 | 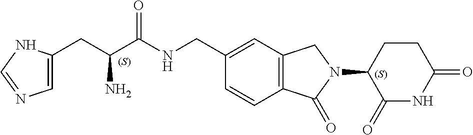 |
| 29 | 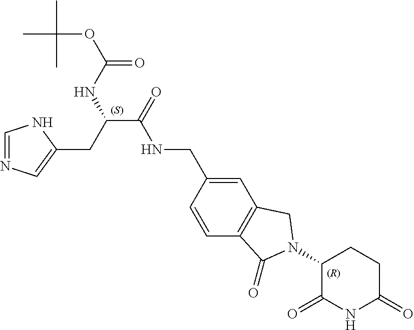 |
| 30 | 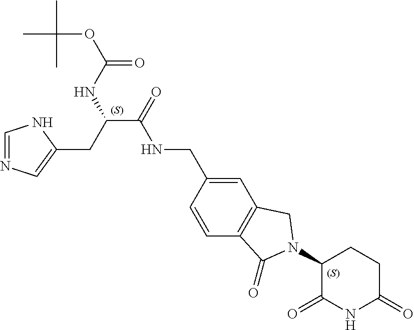 |
| 31 | 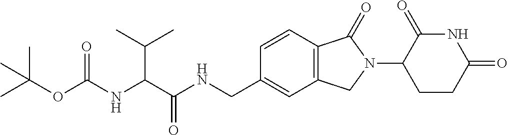 [SMILES: CC(C)C(NC(=O)OC(C)(C)C)C(=O)NCc1ccc2c(c1)CN(C1CCC(=O)NC1=O)C2=O] |
| 32 | 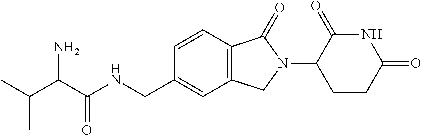 [SMILES: CC(C)C(N)C(=O)NCc1ccc2c(c1)CN(C1CCC(=O)NC1=O)C2=O] |
| 33 | 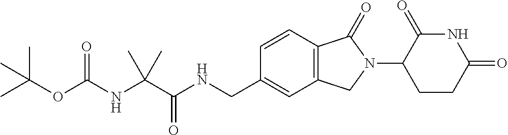 [SMILES: CC(C)(C)OC(=O)NC(C)(C)C(=O)NCc1ccc2c(c1)CN(C1CCC(=O)NC1=O)C2=O] |
| 34 | 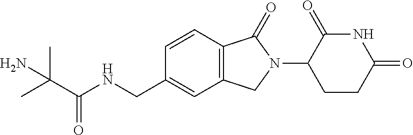 [SMILES: CC(C)(N)C(=O)NCc1ccc2c(c1)CN(C1CCC(=O)NC1=O)C2=O] |
| 35 | 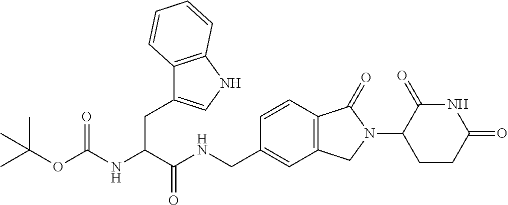 [SMILES: CC(C)(C)OC(=O)NC(Cc1c[nH]c2ccccc12)C(=O)NCc1ccc2c(c1)CN(C1CCC(=O)NC1=O)C2=O] |
| 36 | 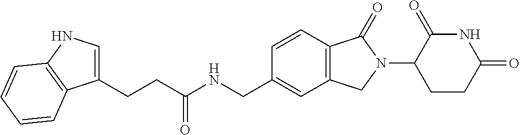 [SMILES: O=C(CCc1c[nH]c2ccccc12)NCc1ccc2c(c1)CN(C1CCC(=O)NC1=O)C2=O] |
| 37 | 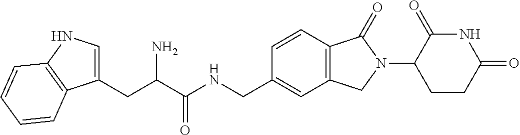 [SMILES: NC(Cc1c[nH]c2ccccc12)C(=O)NCc1ccc2c(c1)CN(C1CCC(=O)NC1=O)C2=O] |
| 38 | 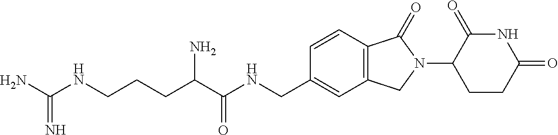 [SMILES: N=C(N)NCCCC(N)C(=O)NCc1ccc2c(c1)CN(C1CCC(=O)NC1=O)C2=O] |
| 39 | 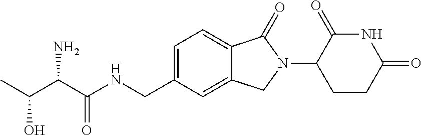 [SMILES: C[C@@H](O)[C@H](N)C(=O)NCc1ccc2c(c1)CN(C1CCC(=O)NC1=O)C2=O] |
| 40 | 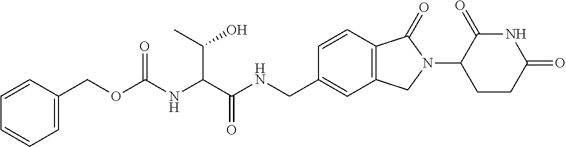 [SMILES: C[C@H](O)C(NC(=O)OCc1ccccc1)C(=O)NCc1ccc2c(c1)CN(C1CCC(=O)NC1=O)C2=O] |
| 41 | 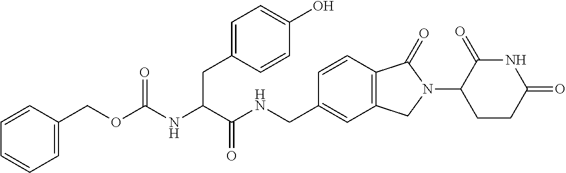 [SMILES: O=C1CCC(N2Cc3cc(CNC(=O)C(Cc4ccc(O)cc4)NC(=O)OCc4ccccc4)ccc3C2=O)C(=O)N1] |
| 42 | 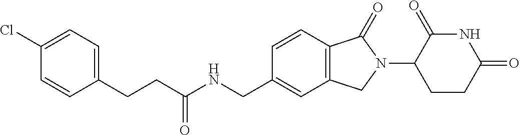 [SMILES: O=C(CCc1ccc(Cl)cc1)NCc1ccc2c(c1)CN(C1CCC(=O)NC1=O)C2=O] |
| 43 | 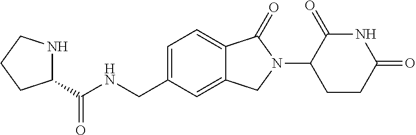 [SMILES: O=C1CCC(N2Cc3cc(CNC(=O)[C@@H]4CCCN4)ccc3C2=O)C(=O)N1] |
| 44 | 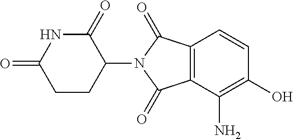 [SMILES: Nc1c(O)ccc2c1C(=O)N(C1CCC(=O)NC1=O)C2=O] |
| 50 | 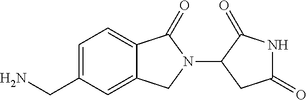 [SMILES: NCc1ccc2c(c1)CN(C1CC(=O)NC1=O)C2=O] |
| 51 | 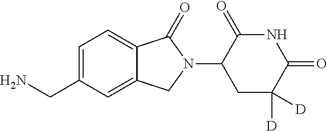 [SMILES: [2H]C1([2H])CC(N2Cc3cc(CN)ccc3C2=O)C(=O)NC1=O] |
| 52 | 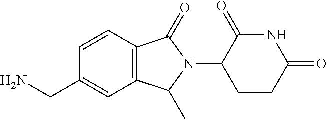 [SMILES: CC1c2cc(CN)ccc2C(=O)N1C1CCC(=O)NC1=O] |
| 53 | 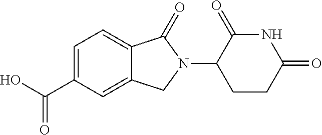 [SMILES: O=C1CCC(N2Cc3cc(C(=O)O)ccc3C2=O)C(=O)N1] |
| 54 | 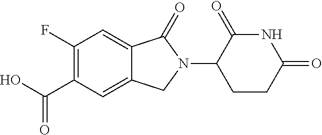 [SMILES: O=C1CCC(N2Cc3cc(C(=O)O)c(F)cc3C2=O)C(=O)N1] |
| 48 | 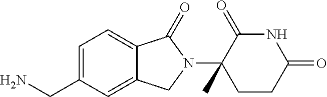 [SMILES: C[C@]1(N2Cc3cc(CN)ccc3C2=O)CCC(=O)NC1=O] |
| 49 | 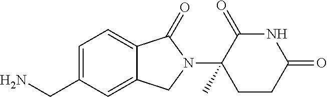 [SMILES: C[C@@]1(N2Cc3cc(CN)ccc3C2=O)CCC(=O)NC1=O] |

and pharmaceutically acceptable salts, esters, optically active isomers, racemates, solvates, amino acid conjugates, or prodrugs thereof.

In some embodiments of any of the fourth to sixth aspects, R^{c} is NHR².

In some embodiments of any of the fourth to sixth aspects, R² is selected from H, —COR³, and —COOR³.

In some embodiments of any of the fourth to sixth aspects, R³ is C₁-C₁₀ alkyl substituted with one or more R⁴.

In some embodiments of any of the fourth to sixth aspects, each R⁴ is independently selected from NH₂, OCOR⁵, indole, and 6-membered aryl substituted with one or more —OCO(C₁-C₄ alkyl); wherein R⁴ is not X.

In some embodiments of any of the fourth to sixth aspects, the compound is selected from compounds 51, 2, 22, 3, 24, 6, 23, 52, 37, and 1:

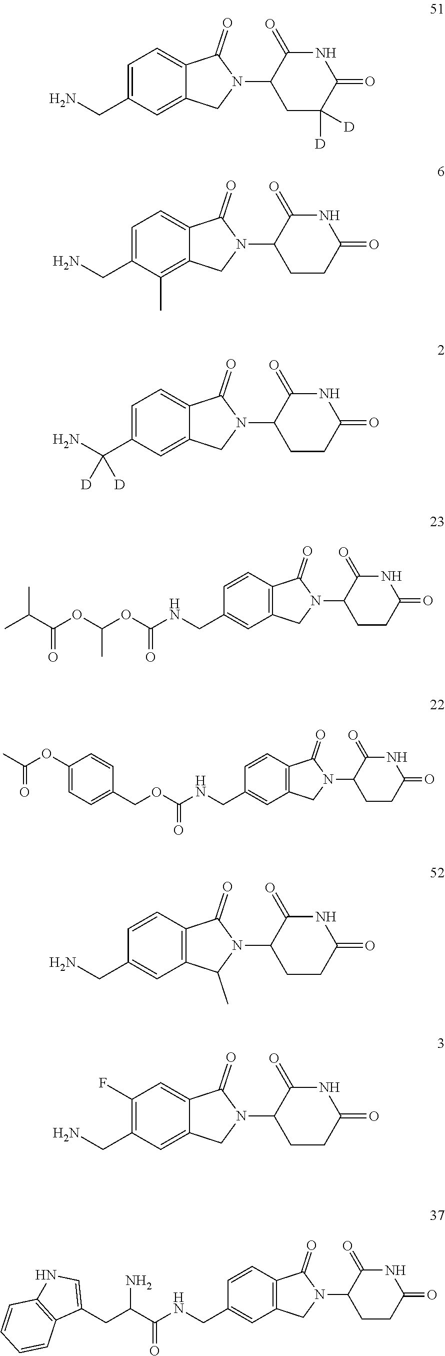

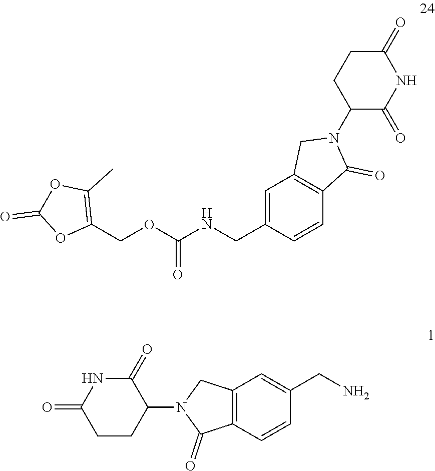 [SMILES: Cc1oc(=O)oc1COC(=O)NCc1ccc2c(c1)CN(C1CCC(=O)NC1=O)C2=O ; NCc1ccc2c(c1)CN(C1CCC(=O)NC1=O)C2=O]

In some embodiments of any of the fourth to sixth aspects, the cancer is hepatocellular carcinoma, neuroblastoma, leukemia, acute myeloid leukemia (AML), acute promyelocytic leukemia (APL), multiple myeloma, breast cancer, prostate cancer, bladder cancer, kidney cancer, muscle cancer, ovary cancer, skin cancer, pancreas cancer, breast cancer, colon cancer, hematological cancer, cancer of a connective tissue, placenta cancer, bone cancer, uterus cancer, cervical cancer, choriocarcinoma, endometrial cancer, gastric cancer, or lung cancer. In some embodiments, the cancer is hepatocellular carcinoma, neuroblastoma, leukemia, prostate cancer, or multiple myeloma.

In some embodiments of any of the fourth to sixth aspects, the cancer is hepatocellular carcinoma. In some such embodiments, the compound is:
(a) selected from compounds 6, 3, 36, 42, 26, 23, 24, 1, 52, 28, 27, 37, 39, 38, and 5;
(b) selected from compounds 6, 3, 36, 42, 26, 23, 24, 1, and 52.

In some embodiments of any of the fourth to sixth aspects, the cancer is neuroblastoma. In some such embodiments, the compound is selected from compounds 3, 36, 42, 37, 28, 27, and 1.

In some embodiments of any of the fourth to sixth aspects, the cancer is leukemia. In some such embodiments, the compound is selected from compounds 3, 36, 42, 37, 28, 27, 24, and 1.

In some embodiments of any of the fourth to sixth aspects, the method of treating cancer further comprises administering a second cancer therapy to the subject. In some embodiments, the second cancer therapy is chemotherapy, radiotherapy or immunotherapy. In some embodiments, the second cancer therapy comprises administration of an agent selected from a therapeutic antibody that specifically binds to a cancer antigen, a hematopoietic growth factor, a cytokine, anti-cancer agent, an antibiotic, a cox-2 inhibitor, an immunomodulatory agent, an immunosuppressive agent, a corticosteroid or a pharmacologically active mutant or derivative thereof.

In some embodiments of any of the fourth to sixth aspects, the method comprises oral administration of the compound or the pharmaceutical composition to the subject.

In some embodiments of any of the fourth to sixth aspects, the cancer is associated with one or more proteins selected from the group consisting of SALL4 or GSPT1.

In some embodiments of any of the first to sixth aspects, the compound is of formula (Ia) or formula (II)

In some embodiments of any of the first to sixth aspects, the compound is of formula (Ib).

In some embodiments of any of the first to sixth aspects, the compound is of formula (Ic).

In some embodiments of any of the first to sixth aspects, the compound is of formula (Ia) or formula (Ic).

In some embodiments of any of the first to sixth aspects, the compound is of formula (II).

In some embodiments of any of the first to sixth aspects, L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″. In some embodiments, L is alkyl, benzyl, —CH₂OC(O)Me, or —CH₂OC(O)^{t}Bu, In other embodiments, L is hydrogen.

In some embodiments of any of the first to sixth aspects, n is 1. In other embodiments, n is 0.

In some embodiments of any of the first to sixth aspects, each R¹⁴ is deuterium. In other embodiments, each R¹⁴ is hydrogen.

In some embodiments of any of the first to sixth aspects, R¹⁵ is deuterium. In other embodiments, R¹⁵ is hydrogen.

In some embodiments of any of the first to sixth aspects, R^{e} is X.

In some embodiments of any of the first to sixth aspects, R¹ is selected from H and methyl. In some embodiments, R¹ is H.

In some embodiments of any of the first to sixth aspects R² is selected from H, methyl, —COR³, and —COOR³. In some embodiments, R² is H or methyl. In some embodiments, R² is —COR³ or —COOR³.

In some embodiments of any of the first to sixth aspects, R¹ is H and R² is H. In other embodiments, R¹ is methyl and R² is methyl. In other embodiments, R¹ is H and R² is —COR³ or —COOR³.

In some embodiments of any of the first to sixth aspects, administration of the compound or pharmaceutical composition to a subject reduces levels of a target protein in the subject.

In some embodiments, the target protein is selected from SALL-4 and GSPT1.

In some embodiments of any of the first to sixth aspects, administration of the compound or pharmaceutical composition to the subject induces minimal reduction or substantially no reduction in IKZF1 or IKZF3 protein levels.

### BRIEF DESCRIPTION OF THE DRAWINGS

FIG. 1 shows representative results from the SALL4 degradation assay in the Kelly cell line. Cells were treated with the compounds: 1 and 44 of the invention and reference compounds Thalidomide and Lenalidomide, at the concentrations of 0.01-1 μM for 24 h.

FIG. 2 shows SALL4 degradation in the Kelly cell line—Time Course. Cells were treated with the compounds: Lenalidomide, 1 and 44 at the concentration of 0.1 μM for 3, 6, 12, 24, 48 and 72 h. A) WB membrane, B) densitometry percent of DMSO control, normalized to the loading control.

FIG. 3 shows representative results from the GSPT1 degradation assay in the Hep3B cell line. Cells were treated with the compounds: 52, 5, 7, and 54 of the invention at the concentrations of 10 μM for 24 h.

FIG. 4 shows representative results from the Ikaros (IKZF1) degradation assay in the H929 cell line. A) Cells were treated with the compounds: 1, 44, 28, and 27 of the present invention and reference compound Lenalidomide at the concentrations 1 and 10 μM for 24 h. B) Cells were treated with the compounds: 4, 52, 5, 7, and 54 and the reference compounds 100, CC-90009 and Pomalidomide at the concentration of 10 μM for 24 h.

FIG. 5 shows representative results from the Aiolos (IKZF3) degradation assay in the H929 cell line. Cells were treated with the compounds: 1, 44, 28, and 27 of the present invention and reference compound Lenalidomide at the concentrations 1 and 10 μM for 24 h.

FIG. 6 shows the influence of various compounds on cell viability. Luminescence (RLU) values normalized to DMSO control. A) Hep3B cells were treated with compounds 1, 2, 3, 6, 23, 37, and 52 of the present invention at the range of concentrations 0.001-50 μM for 72 h B) H929 cells were treated with compounds 1, 3, 37, 52 of the present invention and reference compounds CC-90009 and pomalidomide at the range of concentrations 0.001-50 μM for 72 h C) SNU-398 cells were treated with compound 3 of the present invention and reference compound CC-90009 at the range of concentrations 0.001-50 μM for 72 h.

FIG. 7 shows the influence of various compounds on cell survival. A) Kelly and B) Hep3B cells were treated with compounds: Lenalidomide or 1 at the range of concentrations 0.1-10 μM. The crystal violet staining was performed after 9-10 days of culture.

### DETAILED DESCRIPTION OF THE INVENTION

The present invention provides compounds of formula (Ia), (Ib), (Ic) and (II) as defined above, and pharmaceutical compositions comprising these compounds.

The invention also provides a method for treating cancer, which comprises administering a compound or pharmaceutical composition of the present invention to a subject in need thereof.

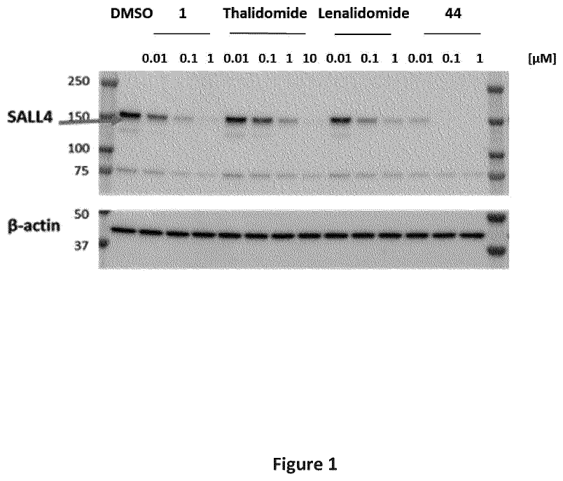

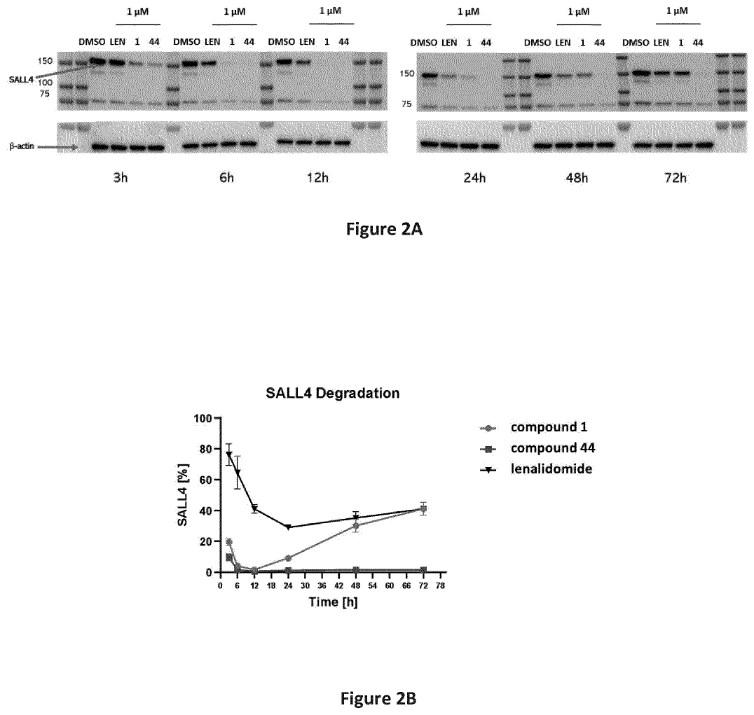

The compounds of the invention induce potent degradation of SALL4 protein in the Kelly (neuroblastoma) cell line in a broad concentration range (see FIG. 1). The compounds of the present invention may therefore be useful as anti-cancer drug candidates. The compounds of the present invention have a unique degradation profile, as they induce potent degradation of selected oncogenic proteins, for example SALL4 protein and GSPT1 protein but are inactive or less potent against Ikaros (IKZF1) and Aiolos (IKZF3). The unique degradation profile of the compounds is surprising, given the profile of existing degraders, such as Thalidomide and Lenalidomide (see FIGS. 1-5). Moreover, the dynamics of SALL4 degradation was assessed (see FIG. 2). The compounds of the present invention degraded SALL4 more rapidly and effectively than lenalidomide, which suggests that the inventive compounds could be administered at lower doses than the reference compounds.

The compounds of the present invention potently inhibit growth of several cancer types: hepatocellular carcinoma (HEP3B, SNU-398), neuroblastoma (Kelly), leukemia (KG-1, KG-1a, UOC-M1, MOLT-3, MOLT-4, MOLM-13, MOLM-1, MOLM-6) prostate cancer (22Rv1), multiple myeloma (MOLP-2). Simultaneously, the compounds of the present invention do not exhibit activity towards H929 and various other cell lines (Table 10 and 12) which makes them unique in comparison to the known compounds CC-90009, Lenalidomide, Pomalidomide, CC-122, and CC-220. This surprising effect makes the compounds clinically attractive due to their enhanced selectivity that will likely correspond to a therapeutic window in particular cancer types, such as HCC.

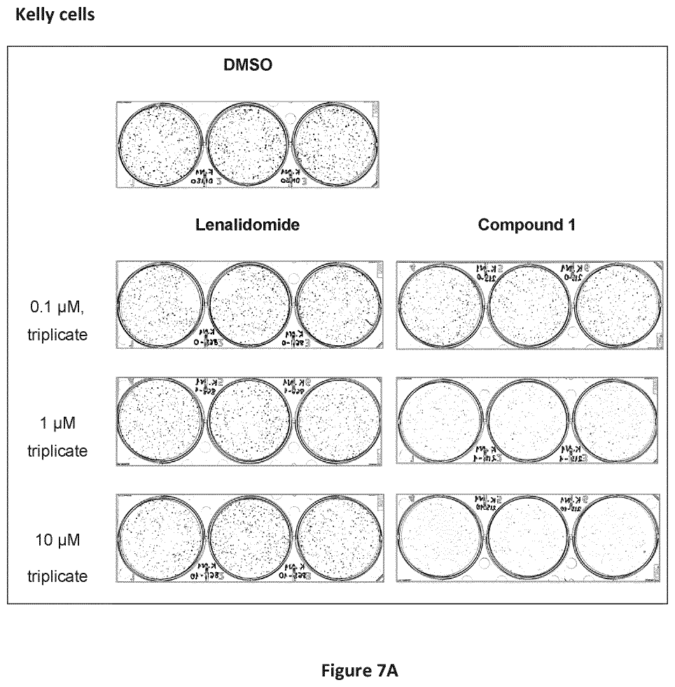

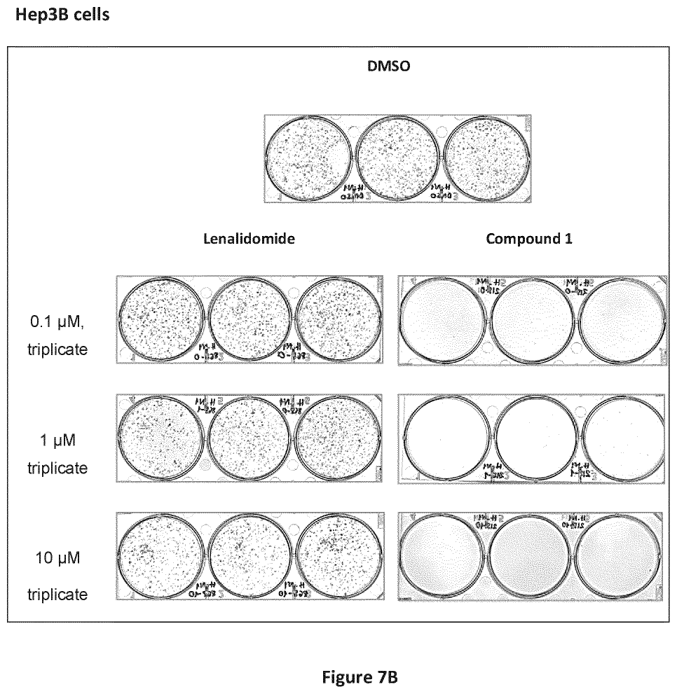

Further, the ability of the compounds of the present invention to influence the survival of cancer cell lines was evaluated (see FIGS. 7A and 7B). In the SALL4 expressing cell lines (Kelly, Hep3B), the survival of the cells was impaired by the compound of the present invention, while lenalidomide presented no activity.

The compounds of the present invention also exhibit particularly favorable pharmacokinetics.

Compounds of the present invention (or for use in accordance with the present invention) include:

| Compound | |
|---|---|
| ID | Structure |
| 1 | 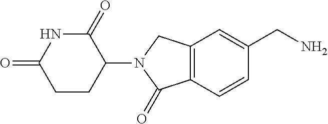 [SMILES: NCc1ccc2c(c1)CN(C1CCC(=O)NC1=O)C2=O] |
| 2 | 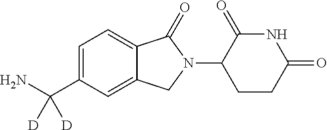 [SMILES: [2H]C([2H])(N)c1ccc2c(c1)CN(C1CCC(=O)NC1=O)C2=O] |
| 3 | 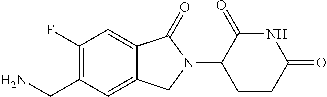 [SMILES: NCc1cc2c(cc1F)C(=O)N(C1CCC(=O)NC1=O)C2] |
| 4 | 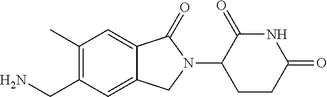 [SMILES: Cc1cc2c(cc1CN)CN(C1CCC(=O)NC1=O)C2=O] |
| 5 | 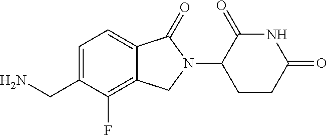 [SMILES: NCc1ccc2c(c1F)CN(C1CCC(=O)NC1=O)C2=O] |
| 6 | 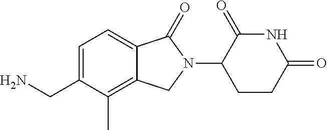 [SMILES: Cc1c(CN)ccc2c1CN(C1CCC(=O)NC1=O)C2=O] |
| 7 | 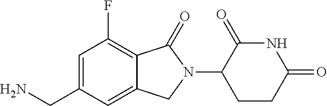 [SMILES: NCc1cc(F)c2c(c1)CN(C1CCC(=O)NC1=O)C2=O] |
| 8 | 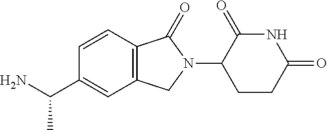 [SMILES: C[C@H](N)c1ccc2c(c1)CN(C1CCC(=O)NC1=O)C2=O] |
| 9 | 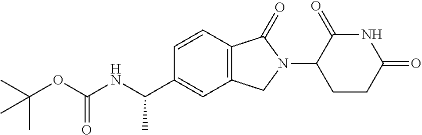 [SMILES: C[C@H](NC(=O)OC(C)(C)C)c1ccc2c(c1)CN(C1CCC(=O)NC1=O)C2=O] |
| 10 | 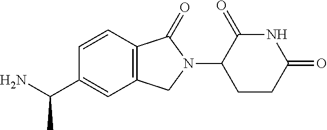 [SMILES: C[C@@H](N)c1ccc2c(c1)CN(C1CCC(=O)NC1=O)C2=O] |
| 11 | 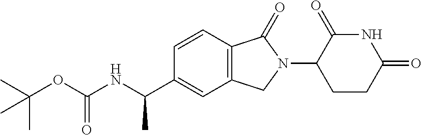 [SMILES: C[C@@H](NC(=O)OC(C)(C)C)c1ccc2c(c1)CN(C1CCC(=O)NC1=O)C2=O] |
| 12 | 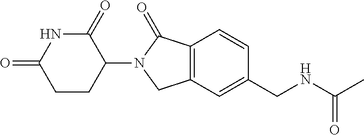 [SMILES: CC(=O)NCc1ccc2c(c1)CN(C1CCC(=O)NC1=O)C2=O] |
| 13 | 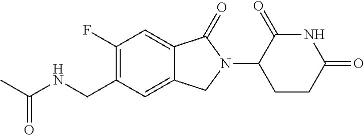 [SMILES: CC(=O)NCc1cc2c(cc1F)C(=O)N(C1CCC(=O)NC1=O)C2] |
| 14 | 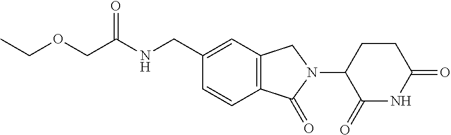 [SMILES: CCOCC(=O)NCc1ccc2c(c1)CN(C1CCC(=O)NC1=O)C2=O] |
| 15 | 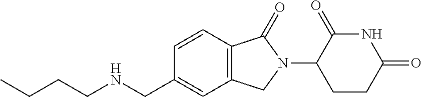 [SMILES: CCCCNCc1ccc2c(c1)CN(C1CCC(=O)NC1=O)C2=O] |
| 16 | 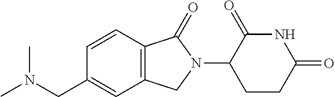 [SMILES: CN(C)Cc1ccc2c(c1)CN(C1CCC(=O)NC1=O)C2=O] |
| 17 | 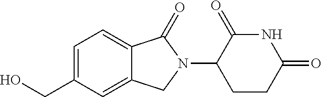 [SMILES: O=C1CCC(N2Cc3cc(CO)ccc3C2=O)C(=O)N1] |
| 18 | 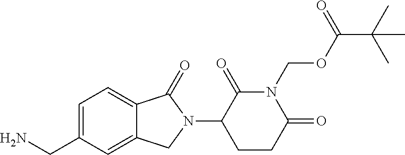 [SMILES: CC(C)(C)C(=O)OCN1C(=O)CCC(N2Cc3cc(CN)ccc3C2=O)C1=O] |
| 19 | 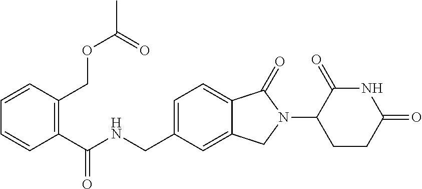 [SMILES: CC(=O)OCc1ccccc1C(=O)NCc1ccc2c(c1)CN(C1CCC(=O)NC1=O)C2=O] |
| 20 | 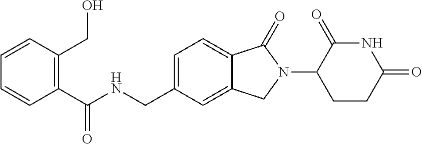 [SMILES: O=C1CCC(N2Cc3cc(CNC(=O)c4ccccc4CO)ccc3C2=O)C(=O)N1] |
| 21 | 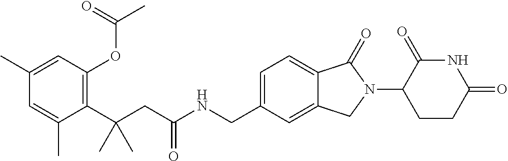 [SMILES: CC(=O)Oc1cc(C)cc(C)c1C(C)(C)CC(=O)NCc1ccc2c(c1)CN(C1CCC(=O)NC1=O)C2=O] |
| 22 | 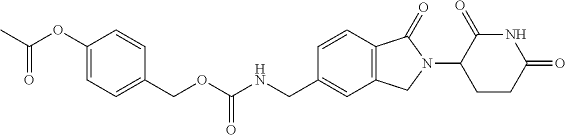 [SMILES: CC(=O)Oc1ccc(COC(=O)NCc2ccc3c(c2)CN(C2CCC(=O)NC2=O)C3=O)cc1] |
| 23 | 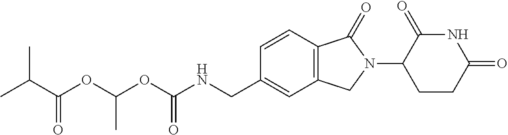 [SMILES: CC(OC(=O)NCc1ccc2c(c1)CN(C1CCC(=O)NC1=O)C2=O)OC(=O)C(C)C] |
| 24 | 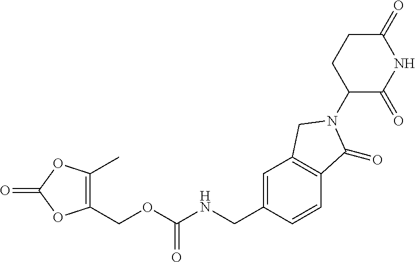 [SMILES: Cc1oc(=O)oc1COC(=O)NCc1ccc2c(c1)CN(C1CCC(=O)NC1=O)C2=O] |
| 25 | 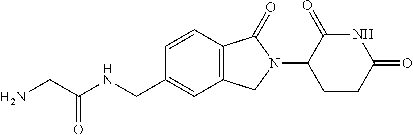 [SMILES: NCC(=O)NCc1ccc2c(c1)CN(C1CCC(=O)NC1=O)C2=O] |
| 26 | 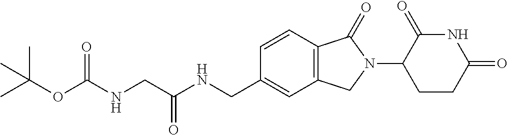 [SMILES: CC(C)(C)OC(=O)NCC(=O)NCc1ccc2c(c1)CN(C1CCC(=O)NC1=O)C2=O] |
| 27 | 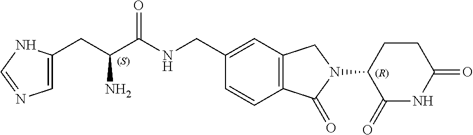 |
| 28 | 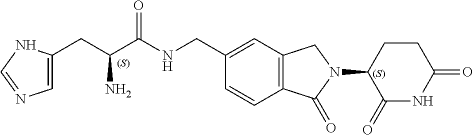 |
| 29 | 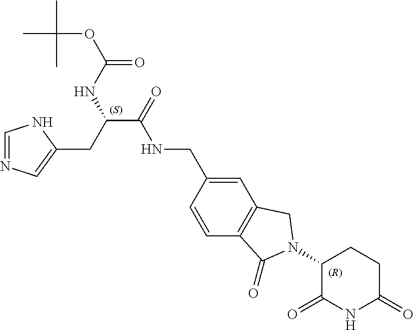 |
| 30 | 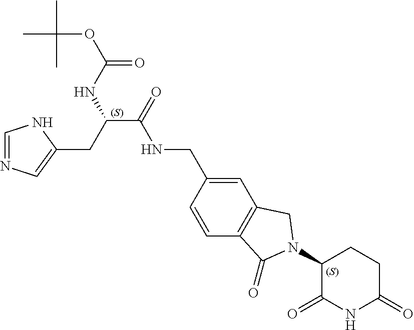 |
| 31 | 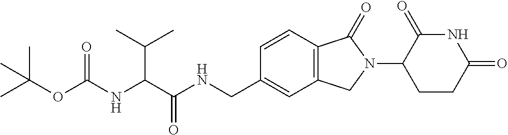 [SMILES: CC(C)C(NC(=O)OC(C)(C)C)C(=O)NCc1ccc2c(c1)CN(C1CCC(=O)NC1=O)C2=O] |
| 32 | 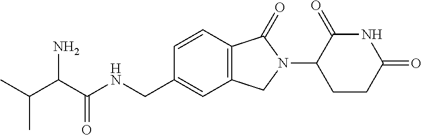 [SMILES: CC(C)C(N)C(=O)NCc1ccc2c(c1)CN(C1CCC(=O)NC1=O)C2=O] |
| 33 | 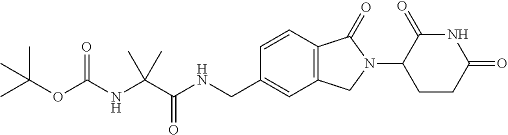 [SMILES: CC(C)(C)OC(=O)NC(C)(C)C(=O)NCc1ccc2c(c1)CN(C1CCC(=O)NC1=O)C2=O] |
| 34 | 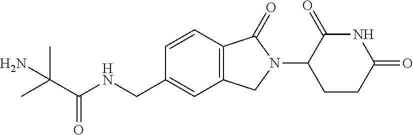 [SMILES: CC(C)(N)C(=O)NCc1ccc2c(c1)CN(C1CCC(=O)NC1=O)C2=O] |
| 35 | 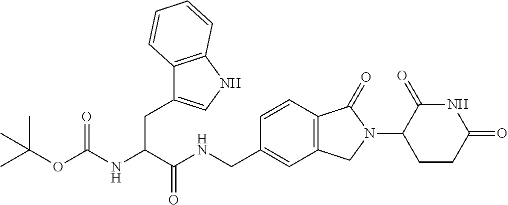 [SMILES: CC(C)(C)OC(=O)NC(Cc1c[nH]c2ccccc12)C(=O)NCc1ccc2c(c1)CN(C1CCC(=O)NC1=O)C2=O] |
| 36 | 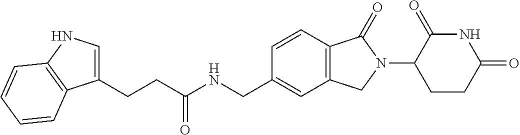 [SMILES: O=C(CCc1c[nH]c2ccccc12)NCc1ccc2c(c1)CN(C1CCC(=O)NC1=O)C2=O] |
| 37 | 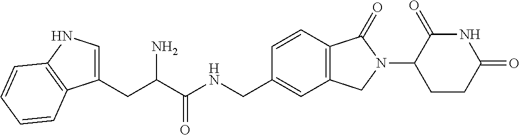 [SMILES: NC(Cc1c[nH]c2ccccc12)C(=O)NCc1ccc2c(c1)CN(C1CCC(=O)NC1=O)C2=O] |
| 38 | 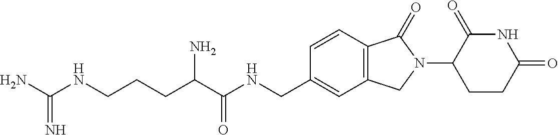 [SMILES: N=C(N)NCCCC(N)C(=O)NCc1ccc2c(c1)CN(C1CCC(=O)NC1=O)C2=O] |
| 39 | 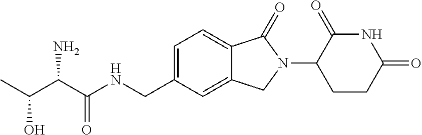 [SMILES: C[C@@H](O)[C@H](N)C(=O)NCc1ccc2c(c1)CN(C1CCC(=O)NC1=O)C2=O] |
| 40 | 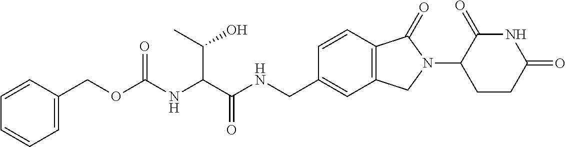 [SMILES: C[C@H](O)C(NC(=O)OCc1ccccc1)C(=O)NCc1ccc2c(c1)CN(C1CCC(=O)NC1=O)C2=O] |
| 41 | 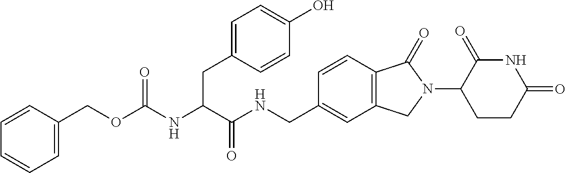 [SMILES: O=C1CCC(N2Cc3cc(CNC(=O)C(Cc4ccc(O)cc4)NC(=O)OCc4ccccc4)ccc3C2=O)C(=O)N1] |
| 42 | 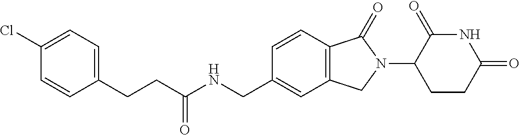 [SMILES: O=C(CCc1ccc(Cl)cc1)NCc1ccc2c(c1)CN(C1CCC(=O)NC1=O)C2=O] |
| 43 | 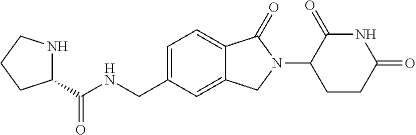 [SMILES: O=C1CCC(N2Cc3cc(CNC(=O)[C@@H]4CCCN4)ccc3C2=O)C(=O)N1] |
| 44 | 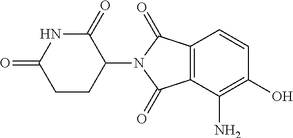 [SMILES: Nc1c(O)ccc2c1C(=O)N(C1CCC(=O)NC1=O)C2=O] |
| 50 | 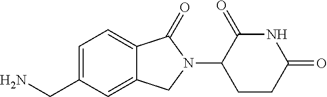 [SMILES: NCc1ccc2c(c1)CN(C1CCC(=O)NC1=O)C2=O] |
| 51 | 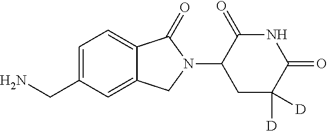 [SMILES: [2H]C1([2H])CC(N2Cc3cc(CN)ccc3C2=O)C(=O)NC1=O] |
| 52 | 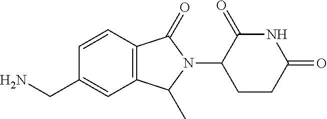 [SMILES: CC1c2cc(CN)ccc2C(=O)N1C1CCC(=O)NC1=O] |
| 53* | 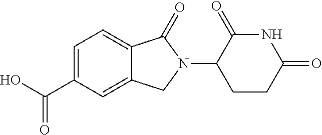 [SMILES: O=C1CCC(N2Cc3cc(C(=O)O)ccc3C2=O)C(=O)N1] |
| 54 | 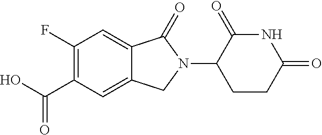 [SMILES: O=C1CCC(N2Cc3cc(C(=O)O)c(F)cc3C2=O)C(=O)N1] |
| 48 | 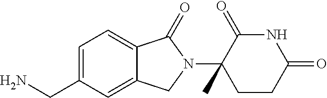 [SMILES: C[C@]1(N2Cc3cc(CN)ccc3C2=O)CCC(=O)NC1=O] |
| 49 | 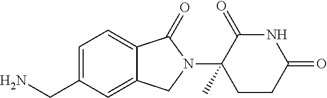 [SMILES: C[C@@]1(N2Cc3cc(CN)ccc3C2=O)CCC(=O)NC1=O] |

*commercially available

The compounds may be in the form of pharmaceutically acceptable salts, esters, optically active isomers, racemates, solvates (e.g. hydrates), amino acid conjugates, or prodrugs thereof.

As used herein, and unless otherwise specified, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. Suitable are hydrochloric, hydrobromic, phosphoric, and sulfuric acids. As used herein, and unless otherwise specified, the term “solvate” means a compound of the present invention or a salt thereof, that further includes a stoichiometric or non-stoichiometric amount of solvent bound by non-covalent intermolecular forces. Where the solvent is water, the solvate is a hydrate.

As used herein, and unless otherwise specified, the term “prodrug” means a derivative of a compound that can hydrolyze, oxidize, or otherwise react under biological conditions {in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO2, —ONO, or —ONO2 moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein, and unless otherwise specified, the term “amino acid conjugate” means a conjugate of a compound (e.g. a compound of formula (I), (II) or (III) as disclosed herein) with any suitable amino acid. For example, suitable amino acids may include (but are not limited to) alanine, valine, isoleucine, leucine, methionine, phenylalanine, tyrosine, tryptophan, serine, threonine, asparagine, glutamine, cysteine, glycine, proline, arginine, histidine, lysine, aspartic acid, and glutamic acid. Particularly suitable amino acids include (but are not limited to) valine, threonine, tyrosine, tryptophan, and arginine.

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “optically active isomer” means a selected isomer of an optically active compounds that exists in at least two isomeric pairs (defined by a chiral center) that rotate the plane polarized light in opposite directions.

As used herein, and unless otherwise specified, the term “stereoisomer” encompasses all enantiomerically/stereomerically pure and enantiomerically/stereomerically enriched compounds of this invention.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “stereomerically enriched” means a composition that comprises greater than about 55% by weight of one stereoisomer of a compound, greater than about 60% by weight of one stereoisomer of a compound, preferably greater than about 70% by weight, more preferably greater than about 80% by weight of one stereoisomer of a compound. As used herein, and unless otherwise indicated, the term “enantiomerically pure” means a stereomerically pure composition of a compound having one chiral center. Similarly, the term “enantiomerically enriched” means a stereomerically enriched composition of a compound having one chiral center.

As used herein, references to a compound inducing “minimal reduction” in levels of a particular protein means a reduction in levels of the protein of less than 25% following 24 hrs incubation of the test cells with 10 μM of the compound. As used herein, references to a compound inducing “substantially no reduction” in levels of a particular protein means a reduction in levels of the protein of less than 25% following 24 hrs incubation of the test cells with 20 μM of the compound.

### EXAMPLES

### General Procedures

The compounds of the present invention are advantageous in terms of their synthetic feasibility. The synthesis of the compounds can be summarized in the following general procedure as set out below:

### Example Method 1: Coupling of Amine with Acid

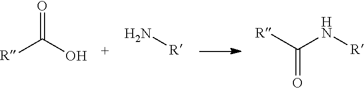

### Reaction Scheme 1: Coupling of Amine with Acid

To a solution of amine (R²NH₂, hydrochloride salt, 1 eq), appropriate acid (R¹COOH in the above reaction scheme) (1.2 eq) and DMAP (0-0.1 eq) in dry DMF under an inert atmosphere were added DIPEA (2.2-5 eq) and HATU (1.2-2.5 eq) in dry DMF. The reaction mixture was stirred overnight at RT. The crude product was purified by preparative HPLC or/and by preparative TLC.

### Example Method 2: Hydrolysis of Lactone

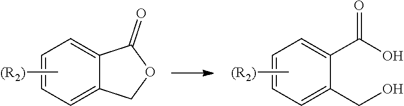

### Reaction Scheme 2. Hydrolysis of Lactone

To a solution of substituted phthalide in a mixture of MeOH, THF (or 2-MeTHF) and H₂O (1:1:1) was added NaOH (4 eq) and the reaction mixture was stirred at RT for 1-18 h. Upon completion, the mixture was diluted with water, acidified with 10% KHSO₄ and the product was extracted using 2-MeTHF. Organic layer was dried over anhydrous Na₂SO₄ and concentrated under reduced pressure to give the product as free acid.

### Example Method 3: Oxidation of Hydroxyacid

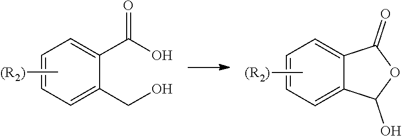

### Reaction Scheme 3. Oxidation of Hydroxyacid

To the suspension of pyridinium chlorochromate (1.5 eq) in anhydrous DCM was added a solution of hydroxyacid (1 eq) in DCM. The reaction mixture was stirred for 1-3 h at RT, diluted with diethyl ether, filtered through Celite and concentrated under reduced pressure. The product was purified by flash column chromatography.

### Example Method 4: Reductive Amination with Amine

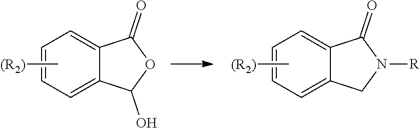

### Reaction Scheme 4. Reductive Amination

To a solution of hydroxyfuranone (1 eq) and amine (R²NH₂, hydrochloride salt, 1.5 eq) in DMF was added NaBH(OAc)₃ (2.5-5 eq) followed by TFA or AcOH (0-50 eq). The reaction mixture was stirred for 18 h at RT, concentrated under reduced pressure and the crude product was purified by flash column chromatography or/and preparative HPLC or/and preparative TLC.

### Example Method 5: Reaction of o-(bromometyl)arylester with amine

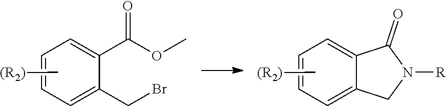

### Reaction Scheme 5. Reaction of o-(bromometyl)arylester with amine

To a mixture of 2-(bromomethyl)aryl ester (1 eq) and amine (R²NH₂, hydrochloride salt, 1.0-1.2 eq) in DMF or ACN, DIPEA (2-5 eq) was added and the mixture was stirred at 90° C. for 6-18 h. The reaction mixture was concentrated under reduced pressure and the crude product was purified by preparative HPLC or/and by preparative TLC.

### Example Method 6: Deprotection of Tert-Butyl Carbamate

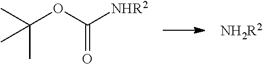

### Reaction Scheme 6. Deprotection of Tert-Butyl Carbamate

### Procedure A

Boc-protected amine was treated with TFA at RT (either neat or as a solution in DCM). The reaction mixture was stirred at RT for 1-24 h and concentrated under reduced pressure to give a product. 0.01 M HCl was added to convert it to HCl salt.

### Procedure B

To the mixture of Boc-protected amine in 1,4-dioxane/H₂O was added concentrated HCl at RT. The reaction mixture was stirred at RT for 1-24 h and concentrated to obtain the product.

### Example 1: Synthesis of 3-(5-(aminomethyl)-1-oxoisoindolin-2-yl)piperidine-2,6-dione (1)

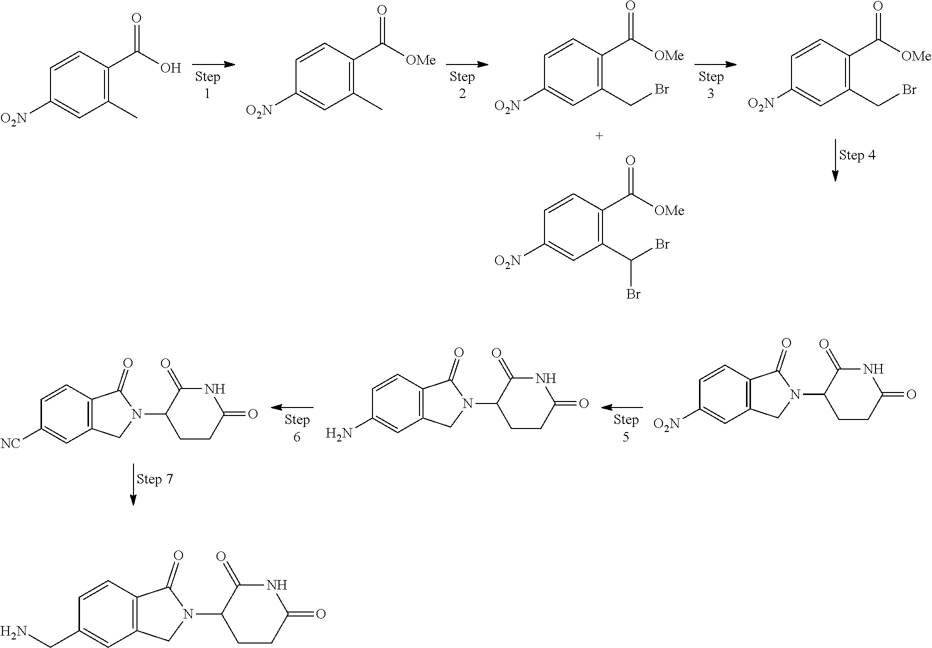

Step 1. 2-Methyl-4-nitrobenzoic acid (181.2 g, 1 mol) was dissolved in MeOH (500 mL), SOCl₂ (119 g, 73 mL, 1 mmol) was added and the mixture was refluxed for 6 h. The solvent was evaporated under reduced pressure. To the residue was added NaHCO_{3(aq)}, the product was extracted into CHCl₃ and concentrated under reduced pressure to obtain 181.5 g of methyl 2-methyl-4-nitrobenzoate (93% yield).

Step 2. Methyl 2-methyl-4-nitrobenzoate (195.2 g, 1 mol) was dissolved in CCl₄ (600 mL), N-Bromosuccinimide (178.0 g, 1 mol) was added and the mixture was stirred for 30 minutes. Catalytic amount of benzoyl peroxide was added and the mixture was refluxed for 3 h, cooled to RT and filtered. The mother liquid was evaporated to yield a mixture of methyl 2-(bromomethyl)-4-nitrobenzoate and methyl 2-(dibromomethyl)-4-nitrobenzoate that was used for the next step without purification.

Step 3. The mixture of methyl 2-(bromomethyl)-4-nitrobenzoate and methyl 2-(dibromomethyl)-4-nitrobenzoate was dissolved in THF (400 mL), diethyl phosphite (1 eq) and DIPEA (1 eq) were added. The reaction mixture was stirred at RT for 12 h. The solvent was removed and the residue was dissolved in EtOAc (300 mL), filtered and the filtrate was washed with water (3×150 mL). The organic layer was separated, dried over anhydrous Na₂SO₄ and evaporated under reduced pressure to obtain 184 g of 2-(bromomethyl)-4-nitrobenzoate (67% yield, for two steps).

Step 4. To a mixture of 2-(bromomethyl)-4-nitrobenzoate (274 g, 1 mol) and 3-aminoglutarimide hydrochloride (198 g, 1.200 mol) in DMF (150 mL), DIPEA (259 g, 350 mL, 2 mol) was added and the mixture was stirred at 90° C. for 6 h, cooled and diluted with water (300 mL). The precipitate was filtered, washed with water and to give 209 g of 3-(5-nitro-1-oxoisoindolin-2-yl)piperidine-2,6-dione (72% yield).

Step 5. 3-(5-nitro-1-oxoisoindolin-2-yl)piperidine-2,6-dione (145 g, 500 mmol) was dissolved in acetic acid (150 mL) with Pd/C 5% (10 mmol) was added and the reaction mixture was stirred in hydrogen atmosphere (30 bars) and at 50° C. for 12 h. The mixture was filtered, washed with EtOAc (2×100 mL) and combined filtrates were evaporated under reduced pressure. Purification of the residue by column chromatography afforded 108 g of 3-(5-amino-1-oxoisoindolin-2-yl)piperidine-2,6-dione (83% yield).

Step 6. A suspension of 3-(5-amino-1-oxoisoindolin-2-yl)piperidine-2,6-dione (25.92 g, 100 mmol) in acetic acid (100 mL) was cooled to +15° C. and treated dropwise with solution of NaNO₂ (8.3 g, 120 mmol) in 50 mL of water. The suspension was stirred at RT for 2 h and then a solution of CuCN (134.5 g, 1.5 mol) and NaCN (49 g, 1 mol) in water (75 mL) was added dropwise over 30 minutes. The mixture was stirred at RT for 3 h and then heated at 60° C. for 2 h. The precipitate was filtered, washed with water and crystallized from DMF/i-PrOH (1:1) to obtain 14 g of 2-(2,6-dioxopiperidin-3-yl)-1-oxoisoindoline-5-carbonitrile (52% yield).

Step 7. To a solution of 2-(2,6-dioxopiperidin-3-yl)-1-oxoisoindoline-5-carbonitrile (2.69 g, 10 mmol) in 1,4-dioxane (150 mL) was added acetic acid (10 mL), mixture was stirred in hydrogen atmosphere (60 bars) at 50° C. for 10 h. Upon completion the mixture was filtered, washed with EtOAc (2×100 mL) and combined filtrates were evaporated under reduced pressure. Purification of the residue by column chromatography afforded 2.08 g of 3-[5-(aminomethyl)-1-oxo-2,3-dihydro-1H-isoindol-2-yl]piperidine-2,6-dione hydrochloride (67% yield).

LCMS: (ESI+) m/z 274.2 [M+H]⁺

¹H NMR (500 MHz, DMSO) δ 11.00 (s, 1H), 8.46 (s, 3H), 7.79 (dd, J=7.8, 0.7 Hz, 1H), 7.73 (s, 1H), 7.64 (dd, J=7.9, 1.5 Hz, 1H), 5.14 (dd, J=13.3, 5.1 Hz, 1H), 4.49 (d, J=17.4 Hz, 1H), 4.36 (d, J=17.4 Hz, 1H), 4.16 (s, 2H), 2.93 (ddd, J=17.3, 13.7, 5.4 Hz, 1H), 2.66-2.57 (m, 1H), 2.48-2.38 (m, 1H), 2.03 (dtd, J=12.7, 5.3, 2.3 Hz, 1H).

### Example 2: Synthesis of 3-(5-(aminomethyl-d₂)-1-oxoisoindolin-2-yl)piperidine-2,6-dione (2)

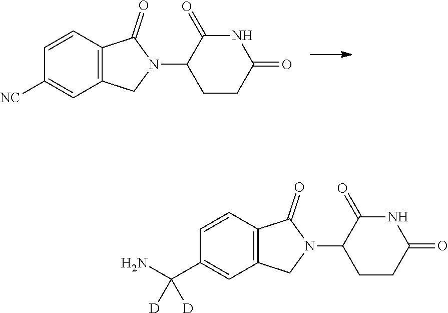 [SMILES: N#Cc1ccc2c(c1)CN(C1CCC(=O)NC1=O)C2=O ; [2H]C([2H])(N)c1ccc2c(c1)CN(C1CCC(=O)NC1=O)C2=O]

A mixture of 2-(2,6-dioxopiperidin-3-yl)-1-oxoisoindoline-5-carbonitrile (0.1 g, 0.37 mmol) and PtO₂ (0.05 g) in i-PrOD (3 mL), DMF (1 mL) and 4 M DCI (0.3 mL) was degassed and the reaction mixture was stirred under D₂ gas (1 bar) at RT for 24 h. The reaction mixture was filtered and concentrated. The crude product was purified by preparative HPLC to give 2 mg of 3-(5-(aminomethyl-d₂)-1-oxoisoindolin-2-yl)piperidine-2,6-dione acetate (20% yield).

LCMS: (ESI+) m/z 276.2 [M+H]⁺

¹H NMR (400 MHz, DMSO) δ 7.65 (d, J=7.5 Hz, 1H), 7.56 (s, 1H), 7.46 (d, J=7.7 Hz, 1H), 5.10 (dd, J=13.3, 5.0 Hz, 1H), 4.43 (d, J=17.0 Hz, 1H), 4.29 (d, J=17.1 Hz, 1H), 2.91 (t, J=13.4 Hz, 1H), 2.62 (s, 1H), 2.40 (d, J=12.4 Hz, 1H), 2.00 (s, 1H). Exchangeable protons not visible.

### Example 3: Synthesis of 3-[5-(aminomethyl)-6-fluoro-1-oxo-2,3-dihydro-1H-isoindol-2-yl]piperidine-2,6-dione (3)

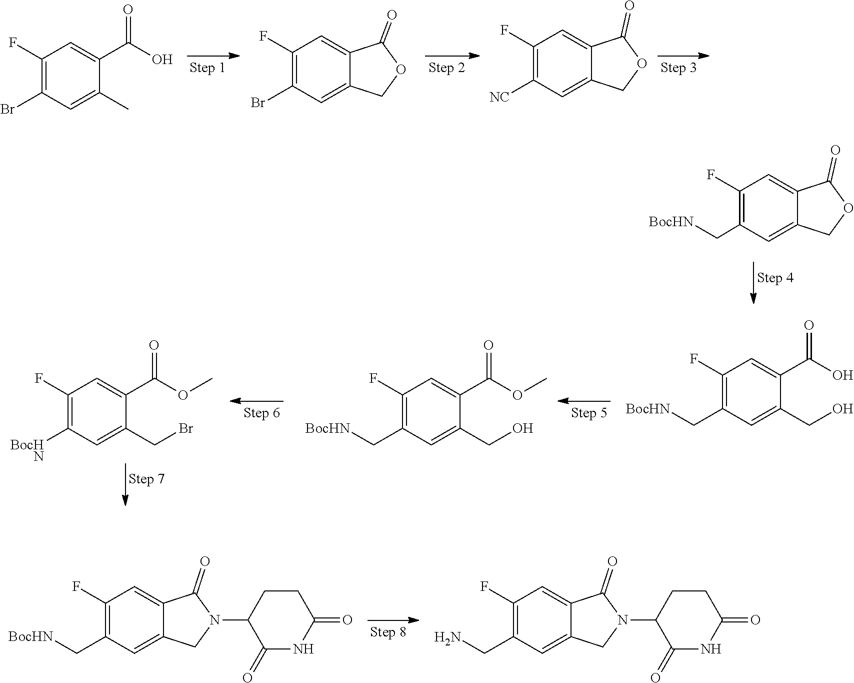

Step 1. To a solution of 4-bromo-5-fluoro-2-methylbenzoic acid (2.0 g, 8.62 mmol) in a mixture of EtOAc/H₂O (25/20 mL) were added NaBrO₃ (4.0 g, 25.86 mmol) and NaHSO₃ (2.7 g, 25.86 mmol) at RT and the reaction mixture was stirred for 48 h. The mixture was washed with water, dried over anhydrous Na₂SO₄, concentrated and purified by flash column chromatography to give 0.8 g of 5-bromo-6-fluoroisobenzofuran-1 (3H)-one (40% yield).

Step 2. To a solution of 5-bromo-6-fluoroisobenzofuran-1 (3H)-one (300 mg, 1.30 mmol) in DMF (7 mL) was added Zn(CN)₂ (384 mg, 3.26 mmol), mixture was purged with argon for 10 minutes. Then to the reaction mixture was added Pd(PPh₃)₄ (227 mg, 0.2 mmol) and it was purged with argon for 10 minutes. The reaction mixture was stirred for 16 h at 90° C. in a sealed tube. After completion of reaction, mixture was filtered through celite bed and washed with EtOAc. Filtrate was diluted with EtOAc and washed with water. The organic layer was dried over anhydrous Na₂SO₄, concentrated under reduced pressure to get the crude material. Crude was purified by column chromatography to give 110 mg of 6-fluoro-1-oxo-1,3-dihydroisobenzofuran-5-carbonitrile (47.6% yield) as light yellow solid.

Step 3. To a solution of 6-fluoro-1-oxo-1,3-dihydroisobenzofuran-5-carbonitrile (630 mg, 3.56 mmol) in ethanol (10 mL), Raney Ni and Boc-anhydride (3.3 mL, 14.26 mmol) were added and reaction mixture was stirred at RT for 16 h under H₂ atmosphere. After completion of reaction, mixture was filtered through celite bed and washed with ethanol. The filtrate was concentrated under reduced pressure and crude was purified by column chromatography to give 600 mg of tert-butyl ((6-fluoro-1-oxo-1,3-dihydroisobenzofuran-5-yl)methyl)carbamate (59.9% yield) as off white solid.

Step 4. To a solution of tert-butyl ((6-fluoro-1-oxo-1,3-dihydroisobenzofuran-5-yl)methyl)carbamate (500 mg, 1.88 mmol) in a mixture of THF (10 mL) and water (8.0 mL) at 0° C. was added NaOH (227 mg, 5.65 mmol). The reaction mixture was then stirred for 16 h at RT. Volatiles were evaporated under reduced pressure, the residue was dissolved in water. It was extracted with EtOAc (20 mL) and then the water phase was acidified with 1(N) HCl while cooling. It was extracted with EtOAc, the combined organic layers were washed with water followed by brine solution. The organic layer was dried over anhydrous Na₂SO₄, concentrated under reduced pressure to give 450 mg 4-(((tert-butoxycarbonyl)amino)methyl)-5-fluoro-2-(hydroxymethyl)benzoic acid (87% yield).

Step 5. To a solution of 4-(((tert-butoxycarbonyl)amino)methyl)-5-fluoro-2-(hydroxymethyl)benzoic acid (600 mg, 2.0 mmol) in methanol (8 mL) and EtOAc (8 mL) was added TMS-diazomethane (11 mL, 20.06 mmol) (2M soln in diethyl ether) dropwise at −10° C. The reaction mixture was then stirred for 3 h at RT. The reaction mixture was then quenched by addition of water and extracted with EtOAc. The combined organic layers were washed with water followed by brine solution. The organic layer was dried over anhydrous Na₂SO₄, concentrated under reduced pressure to give crude 600 mg of methyl 4-(((tert-butoxycarbonyl)amino)methyl)-5-fluoro-2-(hydroxymethyl)benzoate which was used for the next step synthesis without further purification.

Step 6. To a solution of methyl 4-(((tert-butoxycarbonyl)amino)methyl)-5-fluoro-2-(hydroxymethyl)benzoate (900 mg, 2.87 mmol) in THF (20 mL) were added PPh₃ (1.51 g, 5.75 mmol) and carbon CBr₄ (1.91 g, 5.75 mmol) at 0° C. The reaction mixture was then stirred for 16 h at RT under nitrogen atmosphere. The reaction mixture was quenched by addition of water and extracted with EtOAc. The combined organic layers were washed with water followed by brine solution. The organic layer was dried over anhydrous Na₂SO₄, concentrated under reduced pressure to give the crude material. Crude was purified by flash column chromatography to give 360 mg methyl 2-(bromomethyl)-4-(((tert-butoxycarbonyl)amino)methyl)-5-fluorobenzoate (31% yield) as white solid.

Step 7. tert-butyl N-{[2-(2,6-dioxopiperidin-3-yl)-6-fluoro-1-oxo-2,3-dihydro-1H-isoindol-5-yl]methyl}carbamate was synthesized using the general procedure shown in Reaction Scheme 5 and Example method 5, above (73.8% yield), and methyl 2-(bromomethyl)-4-({[(tert-butoxy)carbonyl]amino}methyl)-5-fluorobenzoate (50.0 mg, 0.133 mmol), 3-aminopiperidine-2,6-dione hydrochloride (1.200 eq) as a starting materials.

LCMS: (ESI+) m/z 392.2 [M+H]⁺, (ESI−) m/z 389.9 [M−H]⁻

¹H NMR (500 MHz, DMSO) δ 10.98 (s, 1H), 7.58-7.41 (m, 3H), 5.11 (dd, J=13.4, 5.1 Hz, 1H), 4.45 (d, J=17.2 Hz, 1H), 4.31 (d, J=17.2 Hz, 1H), 4.26 (d, J=6.0 Hz, 2H), 2.91 (ddd, J=17.3, 13.6, 5.4 Hz, 1H), 2.60 (ddd, J=17.4, 4.5, 2.3 Hz, 1H), 2.38 (qd, J=13.2, 4.5 Hz, 1H), 2.06-1.96 (m, 1H), 1.40 (s, 9H).

Step 8. 3-[5-(aminomethyl)-6-fluoro-1-oxo-2,3-dihydro-1H-isoindol-2-yl]piperidine-2,6-dione was synthesized using the general procedure shown in Reaction Scheme 6 and Example method 6, Procedure B, above (97.7% yield), and tert-butyl N-{[2-(2,6-dioxopiperidin-3-yl)-6-fluoro-1-oxo-2,3-dihydro-1H-isoindol-5-yl]methyl}carbamate (7.2 mg, 0.018 mmol) as a starting material.

LCMS: (ESI+) m/z 292.1 [M+H]⁺

¹H NMR (500 MHz, DMSO) δ 11.01 (s, 1H), 8.26 (s, 3H), 7.76 (d, J=6.1 Hz, 1H), 7.65 (d, J=8.6 Hz, 1H), 5.13 (dd, J=13.3, 5.1 Hz, 1H), 4.49 (d, J=17.3 Hz, 1H), 4.35 (d, J=17.3 Hz, 1H), 4.19 (d, J=5.5 Hz, 2H), 2.92 (ddd, J=16.6, 13.4, 5.1 Hz, 1H), 2.59 (d, J=8.7 Hz, 1H), 2.41 (dd, J=13.1, 4.5 Hz, 1H), 2.03 (dtd, J=12.5, 5.3, 2.2 Hz, 1H).

### Example 4: Synthesis of 3-[5-(aminomethyl)-6-methyl-1-oxo-2,3-dihydro-1H-isoindol-2-yl]piperidine-2,6-dione (4)

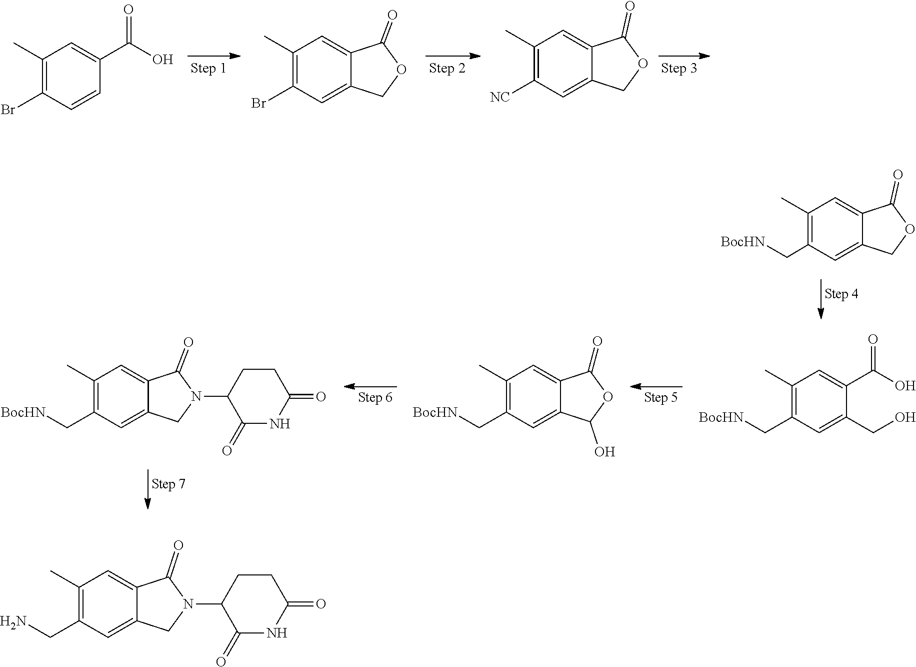

Step 1. To a solution of 4-bromo-3-methylbenzoic acid (2.5 g, 11.628 mmol) in CH₂Br₂ (25 mL) were added K₂HPO₄ (6.07 g, 34.88 mmol) and Pd(OAc)₂ (261 mg, 1.163 mmol). The reaction mixture was stirred at 140° C. for 48 h in a sealed tube under inert atmosphere. The mixture was filtered, concentrated and purified by flash column chromatography to give 750 mg of 5-bromo-6-methylisobenzofuran-1 (3H)-one (28% yield).

Step 2. To a solution of 5-bromo-6-methylisobenzofuran-1 (3H)-one (1.5 g, 6.60 mmol) in DMF (15 mL) was added Zn(CN)₂ (1.933 g, 16.52 mmol) followed by Pd(PPh₃)₄ (0.763 g, 0.661 mmol) and the reaction mixture was heated at 100° C. for 16 h under inert atmosphere. The reaction was quenched with ice water and the product was extracted into EtOAc. The organic layer was dried over Na₂SO₄, concentrated and purified by flash column chromatography to give 900 mg of 6-methyl-1-oxo-1,3-dihydroisobenzofuran-5-carbonitrile (78% yield).

Step 3. To a solution of 6-methyl-1-oxo-1,3-dihydroisobenzofuran-5-carbonitrile (400 mg, 2.30 mmol) in ethanol (5 mL) was added Boc₂O (1.056 mL, 4.598 mmol) followed by Raney Ni (80 mg) and the reaction mixture was stirred under hydrogen atmosphere (1 bar) for 16 h. The reaction mixture was filtered, concentrated under reduced pressure. The crude was purified by flash column chromatography to give 360 mg of tert-butyl ((6-methyl-1-oxo-1,3-dihydroisobenzofuran-5-yl)methyl)carbamate (56% yield).

Step 4. 4-({[(tert-butoxy)carbonyl]amino}methyl)-2-(hydroxymethyl)-5-methylbenzoic acid was synthesized using the general procedure shown in Reaction Scheme 2 and Example method 2, above (82% yield), and tert-butyl N-[(6-methyl-1-oxo-1,3-dihydro-2-benzofuran-5-yl)methyl]carbamate (30.0 mg, 0.108 mmol) as a starting material.

Step 5. tert-butyl N-[(3-hydroxy-6-methyl-1-oxo-1,3-dihydro-2-benzofuran-5-yl)methyl]carbamate was synthesized using the general procedure shown in Reaction Scheme 3 and Example method 3, above (87% yield), and 4-({[(tert-butoxy)carbonyl]amino}methyl)-2-(hydroxymethyl)-5-methylbenzoic acid (26.4 mg, 0.089 mmol) as a starting material.

Step 6. tert-butyl N-{[2-(2,6-dioxopiperidin-3-yl)-6-methyl-1-oxo-2,3-dihydro-1H-isoindol-5-yl]methyl}carbamate was synthesized using the general procedure shown in Reaction Scheme 4 and Example method 4, above (21% yield), and tert-butyl N-[(3-hydroxy-6-methyl-1-oxo-1,3-dihydro-2-benzofuran-5-yl)methyl]carbamate (22.9 mg, 0.070 mmol) as a starting material.

Step 7. 3-[5-(aminomethyl)-6-methyl-1-oxo-2,3-dihydro-1H-isoindol-2-yl]piperidine-2,6-dione was synthesized using the general procedure shown in Reaction Scheme 6 and Example method 6, Procedure A, above (100% yield), and tert-butyl N-{[2-(2,6-dioxopiperidin-3-yl)-6-methyl-1-oxo-2,3-dihydro-1H-isoindol-5-yl]methyl}carbamate (5.1 mg, 0.013 mmol) as a starting material.

LCMS: (ESI+) m/z 288.1 [M+H]⁺

¹H NMR (500 MHz, DMSO, 300 K) δ 10.98 (s, 1H), 8.19 (s, 3H), 7.64 (s, 1H), 7.58 (s, 1H), 5.12 (dd, J=13.3, 5.1 Hz, 1H), 4.44 (d, J=17.1 Hz, 1H), 4.31 (d, J=17.2 Hz, 1H), 4.15 (q, J=5.8 Hz, 2H), 2.92 (ddd, J=17.3, 13.6, 5.4 Hz, 1H), 2.63-2.57 (m, 1H), 2.45 (s, 3H), 2.41 (dd, J=13.0, 4.6 Hz, 1H), 2.02 (ddt, J=12.8, 5.5, 2.8 Hz, 1H).

### Example 5: Synthesis of 3-[5-(aminomethyl)-4-fluoro-1-oxo-2,3-dihydro-1H-isoindol-2-yl]piperidine-2,6-dione (5)

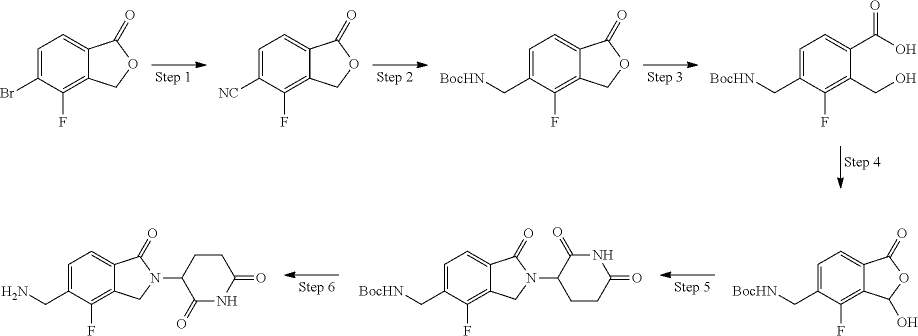

Step 1. To a solution of 5-bromo-4-fluoroisobenzofuran-1 (3H)-one (1.00 g, 4.35 mmol) in DMF (10 mL) was added Zn(CN)₂ (1.24 g, 10.87 mmol) followed by Pd(PPh₃)₄ (0.753 g, 0.652 mmol) and the reaction mixture was heated at 90° C. for 16 h under inert atmosphere. The reaction was quenched with ice water and the product was extracted into EtOAc. The organic layer was dried over Na₂SO₄, concentrated and purified by flash column chromatography to give 500 mg of 4-fluoro-1-oxo-1,3-dihydroisobenzofuran-5-carbonitrile (65% yield).

Step 2. To a solution of 4-fluoro-1-oxo-1,3-dihydroisobenzofuran-5-carbonitrile (500 mg, 2.80 mmol) in ethanol (10 mL) was added Boc₂O (1.29 mL, 5.61 mmol) followed by Raney Ni (100 mg) and the reaction mixture was stirred under hydrogen atmosphere (1 bar) for 16 h. The reaction mixture was filtered, concentrated under reduced pressure. The crude was purified by flash column chromatography to give 395 mg of tert-butyl ((6-methyl-1-oxo-1,3-dihydroisobenzofuran-5-yl)methyl)carbamate (50% yield).

Step 3. 4-({[(tert-butoxy)carbonyl]amino}methyl)-3-fluoro-2-(hydroxymethyl)benzoic acid was synthesized using the general procedure shown in Reaction Scheme 2 and Example method 2, above (98.4% yield), and tert-butyl N-[(4-fluoro-1-oxo-1,3-dihydro-2-benzofuran-5-yl)methyl]carbamate (30.0 mg, 0.107 mmol) as a starting material.

Step 4. tert-butyl N-[(4-fluoro-3-hydroxy-1-oxo-1,3-dihydro-2-benzofuran-5-yl)methyl]carbamate was synthesized using the general procedure shown in Reaction Scheme 3 and Example method 3, above (58% yield), and 4-({[(tert-butoxy)carbonyl]amino}methyl)-3-fluoro-2-(hydroxymethyl)benzoic acid (31.4 mg, 0.105 mmol) as a starting material.

Step 5. tert-butyl N-{[2-(2,6-dioxopiperidin-3-yl)-4-fluoro-1-oxo-2,3-dihydro-1H-isoindol-5-yl]methyl}carbamate was synthesized using the general procedure shown in Reaction Scheme 4 and Example method 4, above (32% yield), and tert-butyl N-[(4-fluoro-3-hydroxy-1-oxo-1,3-dihydro-2-benzofuran-5-yl)methyl]carbamate (20.2 mg, 0.061 mmol) as a starting material.

Step 6. 3-[5-(aminomethyl)-4-fluoro-1-oxo-2,3-dihydro-1H-isoindol-2-yl]piperidine-2,6-dione was synthesized using the general procedure shown in Reaction Scheme 6 and Example method 6, Procedure A, above (100% yield), and tert-butyl N-{[2-(2,6-dioxopiperidin-3-yl)-4-fluoro-1-oxo-2,3-dihydro-1H-isoindol-5-yl]methyl}carbamate (3.8 mg, 0.010 mmol) as a starting material.

LCMS: (ESI+) m/z 333.1 [M+H+ACN]⁺

¹H NMR (500 MHz, DMSO, 300 K) δ 11.01 (s, 1H), 8.28 (s, 3H), 7.73-7.66 (m, 2H), 5.13 (dd, J=13.3, 5.1 Hz, 1H), 4.60 (d, J=17.6 Hz, 1H), 4.43 (d, J=17.5 Hz, 1H), 4.21 (s, 2H), 2.93 (ddd, J=17.3, 13.7, 5.5 Hz, 1H), 2.63-2.56 (m, 1H), 2.42 (dd, J=13.4, 4.5 Hz, 1H), 2.03 (dtd, J=12.6, 5.4, 2.3 Hz, 1H).

### Example 6: Synthesis of 3-[5-(aminomethyl)-4-methyl-1-oxo-2,3-dihydro-1H-isoindol-2-yl]piperidine-2,6-dione (6)

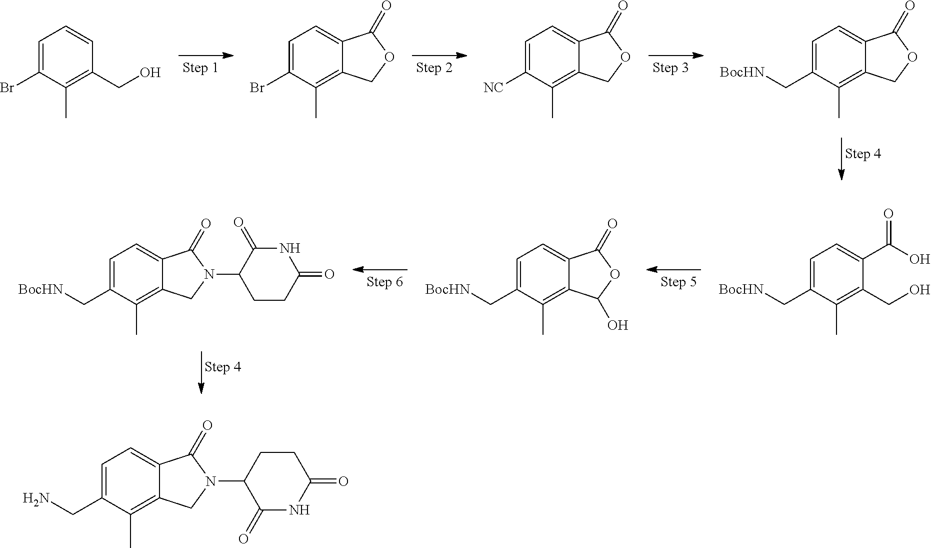

Step 1. To a solution of (3-bromo-2-methylphenyl)methanol (2.3 g, 11.439 mmol) in TFA (10 mL) was added thallium(II) trifluoroacetate (8.081 mg, 14.871 mmol) at 0° C. and the reaction mixture was stirred at RT for 16 h. The reaction mixture was concentrated under reduced pressure, azeotroped with DCE (two times) and dissolved in degassed MeOH (12 mL). MgO (968 mg, 24.023 mmol), LiCl (970 mg, 22.879 mmol) and PdCl₂ (203 mg, 1.144 mmol) were added and the reaction mixture was stirred under CO atmosphere (1 bar) for 4 h. The mixture was filtered, concentrated and purified by flash column chromatography to give 1.55 g of 5-bromo-4-methylisobenzofuran-1 (3H)-one (60% yield).

Step 2. To a solution of 5-bromo-4-methylisobenzofuran-1 (3H)-one (1.5 g, 6.60 mmol) in DMF (15 mL) was added Zn(CN)₂ (1.933 g, 16.52 mmol) followed by Pd(PPh₃)₄ (0.763 g, 6.61 mmol) and the reaction mixture was heated at 100° C. for 16 h under inert atmosphere. The reaction was quenched with ice water and the product was extracted into EtOAc. The organic layer was dried over Na₂SO₄, concentrated and purified by flash column chromatography to give 700 mg of 4-methyl-1-oxo-1,3-dihydroisobenzofuran-5-carbonitrile (61% yield).

Step 3. To a solution of 4-methyl-1-oxo-1,3-dihydroisobenzofuran-5-carbonitrile (1.5 g, 8.67 mmol) in ethanol (15 mL) was added Boc₂O (3.98 mL, 17.34 mmol) followed by Raney Ni (250 mg) and the reaction mixture was stirred under hydrogen atmosphere (1 bar) for 16 h. The reaction mixture was filtered, concentrated and under reduced pressure. The crude was purified by flash column chromatography to give 360 mg of tert-butyl ((4-methyl-1-oxo-1,3-dihydroisobenzofuran-5-yl)methyl)carbamate (45% yield).

Step 4. 4-({[(tert-butoxy)carbonyl]amino}methyl)-2-(hydroxymethyl)-3-methylbenzoic acid was synthesized using the general procedure shown in Reaction Scheme 2 and Example method 2, above (100.0% yield), and tert-butyl N-[(4-methyl-1-oxo-1,3-dihydro-2-benzofuran-5-yl)methyl]carbamate (30.0 mg, 0.108 mmol) as a starting material.

Step 5. tert-butyl N-[(3-hydroxy-4-methyl-1-oxo-1,3-dihydro-2-benzofuran-5-yl)methyl]carbamate was synthesized using the general procedure shown in Reaction Scheme 3 and Example method 3, above (52% yield), and 4-({[(tert-butoxy)carbonyl]amino}methyl)-2-(hydroxymethyl)-3-methylbenzoic acid (36.1 mg, 0.116 mmol) as a starting material.

Step 6. tert-butyl N-{[2-(2,6-dioxopiperidin-3-yl)-4-methyl-1-oxo-2,3-dihydro-1H-isoindol-5-yl]methyl}carbamate was synthesized using the general procedure shown in Reaction Scheme 4 and Example method 4, above (15% yield), and tert-butyl N-[(3-hydroxy-4-methyl-1-oxo-1,3-dihydro-2-benzofuran-5-yl)methyl]carbamate (36.1 mg, 0.064 mmol) as a starting material.

Step 7. 3-[5-(aminomethyl)-4-methyl-1-oxo-2,3-dihydro-1H-isoindol-2-yl]piperidine-2,6-dione was synthesized using the general procedure shown in Reaction Scheme 6 and Example method 6, Procedure A, above (22.6% yield), and tert-butyl N-{[2-(2,6-dioxopiperidin-3-yl)-4-methyl-1-oxo-2,3-dihydro-1H-isoindol-5-yl]methyl}carbamate (11.8 mg, 0.031 mmol) as a starting material.

LCMS: (ESI+) m/z 288.1 [M+H]⁺

¹H NMR (500 MHz, DMSO, 300 K) δ 10.98 (s, 1H), 8.19 (s, 3H), 7.64 (s, 1H), 7.58 (s, 1H), 5.12 (dd, J=13.3, 5.1 Hz, 1H), 4.44 (d, J=17.1 Hz, 1H), 4.31 (d, J=17.2 Hz, 1H), 4.15 (q, J=5.8 Hz, 2H), 2.92 (ddd, J=17.3, 13.6, 5.4 Hz, 1H), 2.63-2.57 (m, 1H), 2.45 (s, 3H), 2.41 (dd, J=13.0, 4.6 Hz, 1H), 2.02 (ddt, J=12.8, 5.5, 2.8 Hz, 1H).

### Example 7: Synthesis of 3-[5-(aminomethyl)-7-fluoro-1-oxo-2,3-dihydro-1H-isoindol-2-yl]piperidine-2,6-dione (7)

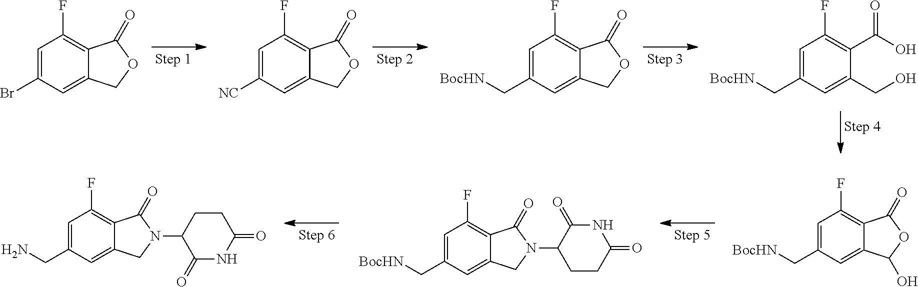

Step 1. To a solution of 5-bromo-7-fluoroisobenzofuran-1 (3H)-one (250 mg, 1.082 mmol) in dioxane (7 mL) was added Zn(CN)₂ (254 mg, 2.165 mmol) followed by Pd₂dba₃ (99 mg g, 0.11 mmol) and Xantphos (94 mg, 0.162 mmol) and the reaction mixture was heated at 100° C. for 16 h under inert atmosphere. The reaction was filtered, concentrated and purified by flash column chromatography to give 100 mg of 7-fluoro-1-oxo-1,3-dihydroisobenzofuran-5-carbonitrile (52% yield).

Step 2. To a solution of 7-fluoro-1-oxo-1,3-dihydroisobenzofuran-5-carbonitrile (500 mg, 2.825 mmol) in ethanol (20 mL) was added Boc₂O (739 mg, 3.39 mmol) followed by Raney Ni (500 mg) and the reaction mixture was stirred under hydrogen atmosphere (1 bar) for 16 h. The reaction mixture was filtered, concentrated and under reduced pressure. The crude was purified by flash column chromatography to give 300 mg of tert-butyl ((7-fluoro-methyl-1-oxo-1,3-dihydroisobenzofuran-5-yl)methyl)carbamate (37% yield).

Step 3. 4-({[(tert-butoxy)carbonyl]amino}methyl)-2-fluoro-6-(hydroxymethyl)benzoic acid was synthesized using the general procedure shown in Reaction Scheme 2 and Example method 2, above (92.4% yield), and tert-butyl N-[(7-fluoro-1-oxo-1,3-dihydro-2-benzofuran-5-yl)methyl]carbamate (30.0 mg, 0.107 mmol) as a starting material.

Step 4. tert-butyl N-[(7-fluoro-3-hydroxy-1-oxo-1,3-dihydro-2-benzofuran-5-yl)methyl]carbamate was synthesized using the general procedure shown in Reaction Scheme 3 and Example method 3, above (77% yield), and 4-({[(tert-butoxy)carbonyl]amino}methyl)-2-fluoro-6-(hydroxymethyl)benzoic acid (29.5 mg, 0.089 mmol) as a starting material.

Step 5. tert-butyl N-{[2-(2,6-dioxopiperidin-3-yl)-7-fluoro-1-oxo-2,3-dihydro-1H-isoindol-5-yl]methyl}carbamate was synthesized using the general procedure shown in Reaction Scheme 4 and Example method 4, above (17.9% yield), and tert-butyl N-[(7-fluoro-3-hydroxy-1-oxo-1,3-dihydro-2-benzofuran-5-yl)methyl]carbamate (20.3 mg, 0.061 mmol) as a starting material.

Step 6. This compound was synthesized using the general procedure shown in Reaction Scheme 6 and Example method 6, Procedure A, above (100.0% yield), and tert-butyl N-{[2-(2,6-dioxopiperidin-3-yl)-7-fluoro-1-oxo-2,3-dihydro-1H-isoindol-5-yl]methyl}carbamate (4.3 mg, 0.011 mmol) as a starting material.

LCMS: (ESI+) m/z 292.1 [M+H]⁺, m/z 333.1 [M+H+ACN]⁺

¹H NMR (500 MHz, DMSO, 300 K) δ 11.00 (s, 1H), 8.25 (s, 3H), 7.50 (s, 1H), 7.41 (d, J=10.3 Hz, 1H), 5.09 (dd, J=13.4, 5.1 Hz, 1H), 4.51 (d, J=17.9 Hz, 1H), 4.37 (d, J=18.0 Hz, 1H), 4.17 (d, J=4.8 Hz, 2H), 2.92 (ddd, J=17.3, 13.6, 5.4 Hz, 1H), 2.63-2.57 (m, 1H), 2.39 (dd, J=13.1, 4.5 Hz, 1H), 2.02 (dtd, J=10.6, 5.4, 2.8 Hz, 1H).

### Example 8: Synthesis of tert-butyl N-[(1S)-1-[2-(2,6-dioxopiperidin-3-yl)-1-oxo-2,3-dihydro-1H-isoindol-5-yl]ethyl]carbamate (9) and 3-{5-[(1S)-1-aminoethyl]-1-oxo-2,3-dihydro-1H-isoindol-2-yl}piperidine-2,6-dione (8)

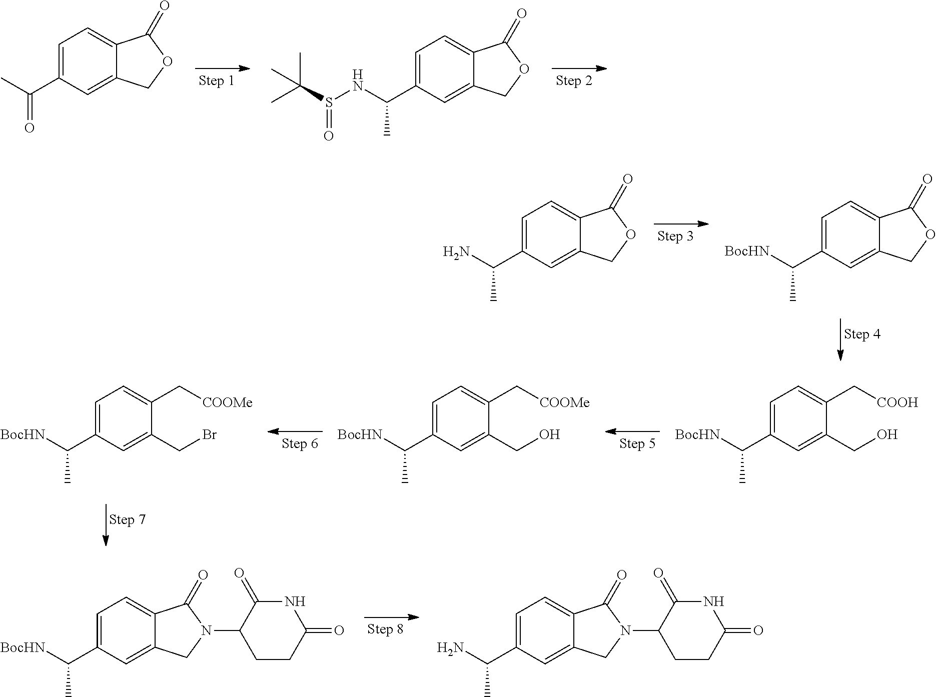

Step 1. To a solution of 5-acetylisobenzofuran-1 (3H)-one (3.0 g, 17.04 mmol) and (S)-2-methylpropane-2-sulfinamide (2.27 g, 18.74 mmol) in THF (50 mL) was added Ti(OEt)₄ (7.14 mL, 34.08 mmol) at 0° C. and the reaction mixture was stirred at 70° C. for 20 h. The reaction mixture was then added dropwise to the suspension of NaBH₄ (2.57 g, 68.16 mmol) in THF at −60° C. and slowly warmed to RT. The reaction mixture was quenched with MeOH (10 mL) and poured into the brine solution, filtered and diluted with water. The product was extracted into extracted with EtOAc. The organic layer was dried over anhydrous Na₂SO₄, concentrated under reduced pressure and purified by flash column chromatography to give 2.8 g of (S)-2-methyl-N—((S)-1-(1-oxo-1,3-dihydroisobenzofuran-5-yl)ethyl)propane-2-sulfinamide (58% yield) as a white solid.

Step 2. To a solution of (S)-2-methyl-N—((S)-1-(1-oxo-1,3-dihydroisobenzofuran-5-yl)ethyl)propane-2-sulfinamide (510 mg, 1.815 mmol) in 1,4-dioxane (2 mL) was added 4M HCl in 1,4-dioxane at 10° C. The reaction mixture was stirred at RT for 1 h and concentrated to give 315 mg (S)-5-(1-aminoethyl)isobenzofuran-1 (3H)-one (97% yield) and forwarded to the next step.

Step 3. To a solution of (S)-5-(1-aminoethyl)isobenzofuran-1 (3H)-one (2.3 g, 12.99 mmol) in THF/H₂O (30/20 mL) were added Boc₂O (4.47 mL, 19.49 mmol) and NaHCO₃ (2.18 g, 25.98 mmol) at 0° C. and the reaction mixture was stirred at RT for 16 h. The product was extracted into EtOAc. The organic layer was dried over anhydrous Na₂SO₄, concentrated under reduced pressure and purified by flash column chromatography to give 1.9 g tert-butyl (S)-(1-(1-oxo-1,3-dihydroisobenzofuran-5-yl)ethyl)carbamate (52% yield) as a white solid.

Step 4. To a solution of tert-butyl (S)-(1-(1-oxo-1,3-dihydroisobenzofuran-5-yl)ethyl)carbamate (1.9 g, 6.85 mol) in THF/H₂O (6/24 mL) was added NaOH (412 mg, 10.29 mmol) at 0° C., reaction mixture was stirred at RT for 1 h. After completion of the reaction, the mixture was acidified (pH˜5) by 10% HCl solution at 0° C. and extracted with EtOAc. The combined organic layers were dried over Na₂SO₄ and concentrated to give 1.75 g (S)-4-(1-((tert-butoxycarbonyl)amino)ethyl)-2-(hydroxymethyl)benzoic acid (88.5% yield) as a white solid.

Step 5. To a solution of (S)-4-(1-((tert-butoxycarbonyl)amino)ethyl)-2-(hydroxymethyl)benzoic acid (1.0 g, 3.37 mmol) in MeOH/EtOAc (6/6 mL) was added TMS-diazomethane (0.912 mL, 16.89 mmol) at −10° C. The reaction mixture was stirred for 30 minutes and quenched with ice water. The product was extracted into EtOAc, dried over Na₂SO₄ and concentrated to give 1.1 g of methyl (S)-4-(1-((tert-butoxycarbonyl)amino)ethyl)-2-(hydroxymethyl)benzoate (crude). The product was used in the next step without further purification.

Step 6. To a solution of methyl (S)-4-(1-((tert-butoxycarbonyl)amino)ethyl)-2-(hydroxymethyl)benzoate (1.1 g, 3.56 mmol, crude) in THF (15 mL) were added PPh₃ (1.76 g, 5.34 mmol) and CBr₄ (1.4 g, 5.34 mmol) at 0° C. and the reaction mixture was stirred at RT for 1 h. The reaction was quenched with ice water and the product was extracted into EtOAc. The organic layer was dried over anhydrous Na₂SO₄, concentrated under reduced pressure and purified by flash column chromatography to give 610 mg of methyl (S)-2-(bromomethyl)-4-(1-((tert-butoxycarbonyl)amino)ethyl)benzoate (36% yield, for two steps) as a white solid.

Step 7. tert-butyl N-[(1S)-1-[2-(2,6-dioxopiperidin-3-yl)-1-oxo-2,3-dihydro-1H-isoindol-5-yl]ethyl]carbamate was synthesized using the general procedure shown in Reaction Scheme 5 and Example method 5, above (67% yield), and methyl 2-(bromomethyl)-4-[(1S)-1-{[(tert-butoxy)carbonyl]amino}ethyl]benzoate (50.0 mg, 0.134 mmol), 3-aminopiperidine-2,6-dione hydrochloride (1.200 eq) as starting materials.

LCMS: (ESI+) m/z 387.8 [M+H]⁺, (ESI−) m/z) 385.9 [M−H]⁻

¹H NMR (500 MHz, DMSO) δ 10.97 (s, 1H), 7.67 (d, J=7.8 Hz, 1H), 7.50 (s, 2H), 7.44 (d, J=7.8 Hz, 1H), 5.10 (dd, J=13.3, 5.1 Hz, 1H), 4.71 (t, J=7.5 Hz, 1H), 4.44 (dd, J=17.2, 4.6 Hz, 1H), 4.30 (dd, J=17.2, 5.0 Hz, 1H), 2.91 (ddd, J=17.2, 13.7, 5.4 Hz, 1H), 2.60 (d, J=17.5 Hz, 1H), 2.40 (td, J=13.2, 4.5 Hz, 1H), 2.03-1.96 (m, 1H), 1.44-1.27 (m, 12H).

Step 8. 3-{5-[(1S)-1-aminoethyl]-1-oxo-2,3-dihydro-1H-isoindol-2-yl}piperidine-2,6-dione was synthesized using the general procedure shown in Reaction Scheme 6 and Example method 6, Procedure B, above (95% yield), and tert-butyl N-{[2-(2,6-dioxopiperidin-3-yl)-6-fluoro-1-oxo-2,3-dihydro-1H-isoindol-5-yl]methyl}carbamate (30.0 mg, 0.077 mmol) as a starting material.

LCMS: (ESI+) m/z 287.8 [M+H]⁺

1H NMR (500 MHz, DMSO) δ 10.99 (s, 1H), 8.54 (s, 3H), 7.80 (dd, J=7.9, 1.6 Hz, 1H), 7.75 (d, J=3.3 Hz, 1H), 7.65 (ddd, J=8.1, 3.4, 1.5 Hz, 1H), 5.13 (dd, J=13.3, 5.1 Hz, 1H), 4.60-4.50 (m, 1H), 4.50-4.44 (m, 1H), 4.35 (dd, J=17.4, 10.0 Hz, 1H), 2.92 (ddd, J=17.3, 13.7, 5.4 Hz, 1H), 2.66-2.56 (m, 1H), 2.46-2.37 (m, 1H), 2.02 (ddq, J=10.3, 5.3, 3.1, 2.6 Hz, 1H), 1.55 (d, J=6.8 Hz, 3H).

### Example 9: Synthesis of tert-butyl N-[(1R)-1-[2-(2,6-dioxopiperidin-3-yl)-1-oxo-2,3-dihydro-1H-isoindol-5-yl]ethyl]carbamate (11) and 3-{5-[(1R)-1-aminoethyl]-1-oxo-2,3-dihydro-1H-isoindol-2-yl}piperidine-2,6-dione (10)

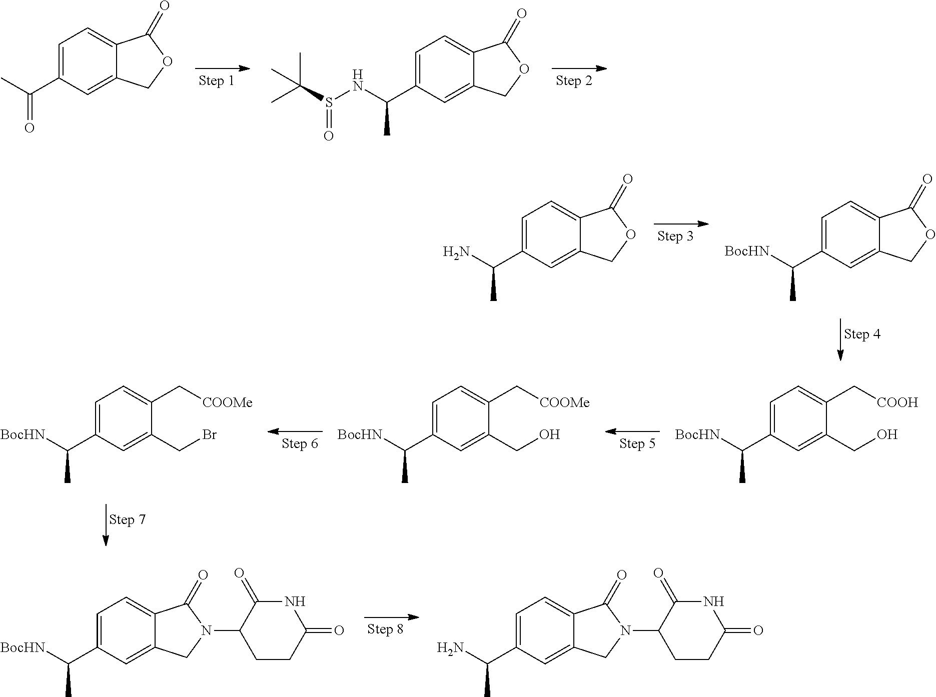

Step 1. To a solution of 5-acetylisobenzofuran-1 (3H)-one (3.5 g, 19.88 mmol) and (R)-2-methylpropane-2-sulfinamide (2.65 mmol) in THF (50 mL) was added Ti(OEt)₄ (8.34 mL, 39.90 mmol) at 0° C. and the reaction mixture was stirred at 70° C. for 20 h. The reaction mixture was then added dropwise to the suspension of NaBH₄ (3.00 g, 79.5 mmol) in THF at −60° C. and slowly warmed to RT. The reaction mixture was quenched with MeOH (10 mL) and poured into the brine solution, filtered and diluted with water. The product was extracted into extracted with EtOAc. The organic layer was dried over anhydrous Na₂SO₄, concentrated under reduced pressure and purified by flash column chromatography to give 2.8 g of (R)-2-methyl-N—((R)-1-(1-oxo-1,3-dihydroisobenzofuran-5-yl)ethyl)propane-2-sulfinamide (50% yield) as a white solid.

Step 2. To a solution of (S)-2-methyl-N—((S)-1-(1-oxo-1,3-dihydroisobenzofuran-5-yl)ethyl)propane-2-sulfinamide (510 mg, 1.815 mmol) in 1,4-dioxane (2 mL) was added 4M HCl in 1,4-dioxane at 10° C. The reaction mixture was stirred at RT for 1 h and concentrated to give to give 2.1 g of (R)-5-(1-aminoethyl)isobenzofuran-1 (3H)-one (95% yield) as a white solid.

Step 3. To a solution of (R)-5-(1-aminoethyl)isobenzofuran-1 (3H)-one (2.1 g, 11.86 mmol) in THF/H₂O (20/20 mL) were added Boc₂0 and NaHCO₃ at 0° C. and the reaction mixture was stirred at RT for 16 h. The product was extracted into EtOAc. The organic layer was dried over anhydrous Na₂SO₄, concentrated under reduced pressure and purified by flash column chromatography to give 2.6 g of tert-butyl (R)-(1-(1-oxo-1,3-dihydroisobenzofuran-5-yl)ethyl)carbamate (79% yield) as a white solid.

Step 4. To a solution of tert-butyl (R)-(1-(1-oxo-1,3-dihydroisobenzofuran-5-yl)ethyl)carbamate (1.6 g, 5.77 mol) in THF/H₂O (6/24 mL) was added NaOH (347 mg, 8.66 mmol) at 0° C., reaction mixture was stirred at RT for 1 h. After completion of the reaction, the reaction mixture was acidified (pH˜5) by 10% HCl solution at 0° C. and extracted with EtOAc. The combined organic layers were dried over Na₂SO₄ and concentrated to give 1.51 g of (R)-4-(1-((tert-butoxycarbonyl)amino)ethyl)-2-(hydroxymethyl)benzoic acid (88.5% yield) as a white solid.

Step 5. To a solution of (R)-4-(1-((tert-butoxycarbonyl)amino)ethyl)-2-(hydroxymethyl)benzoic acid (1.5 g, 5.068 mmol) in MeOH/EtOAc (8/8 mL) was added TMS-diazomethane (12.66 mL, 25.33 mmol) at −10° C. The reaction mixture was stirred for 30 minutes and quenched with ice water. The product was extracted into EtOAc, dried over Na₂SO₄ and concentrated to give 1.67 g of methyl (R)-4-(1-((tert-butoxycarbonyl)amino)ethyl)-2-(hydroxymethyl)benzoate (crude). The crude was forwarded to the next step without purification.

Step 6. To a stirred solution of methyl (R)-4-(1-((tert-butoxycarbonyl)amino)ethyl)-2-(hydroxymethyl)-benzoate (1.67 g, 5.405 mmol, crude) in THF (20 mL) were added PPh₃ (2.68 g, 8.10 mmol) and CBr₄ (2.12 g, 8.10 mmol) at 0° C. and the reaction mixture was stirred at RT for 1 h. The reaction was quenched with ice water and the product was extracted into EtOAc. The organic layer was dried over anhydrous Na₂SO₄, concentrated under reduced pressure and purified by flash column chromatography to give 610 mg of methyl (R)-2-(bromomethyl)-4-(1-((tert-butoxycarbonyl)amino)ethyl)benzoate (30% yield, for two steps) as a white solid.

Step 7. tert-butyl N-[(1R)-1-[2-(2,6-dioxopiperidin-3-yl)-1-oxo-2,3-dihydro-1H-isoindol-5-yl]ethyl]carbamate was synthesized using the general procedure shown in Reaction Scheme 5 and Example method 5, above (66.8% yield), and methyl 2-(bromomethyl)-4-[(1R)-1-{[(tert-butoxy)carbonyl]amino}ethyl]benzoate (50.0 mg, 0.134 mmol), 3-aminopiperidine-2,6-dione hydrochloride (1.200 eq) as starting materials.

LCMS: (ESI+) m/z 388.25 [M+H]⁺

¹H NMR (500 MHz, DMSO) δ 10.98 (s, 1H), 7.68 (d, J=7.8 Hz, 1H), 7.55-7.48 (m, 2H), 7.45 (d, J=7.9 Hz, 1H), 5.11 (dd, J=13.3, 5.1 Hz, 1H), 4.77-4.67 (m, 1H), 4.45 (dd, J=17.2, 4.6 Hz, 1H), 4.31 (dd, J=17.2, 5.1 Hz, 1H), 2.92 (ddd, J=17.2, 13.7, 5.4 Hz, 1H), 2.66-2.57 (m, 1H), 2.40 (qd, J=13.3, 4.5 Hz, 1H), 2.06-1.95 (m, 1H), 1.37 (s, 9H), 1.34 (d, J=7.0 Hz, 3H).

Step 8. 3-{5-[(1R)-1-aminoethyl]-1-oxo-2,3-dihydro-1H-isoindol-2-yl}piperidine-2,6-dione hydrochloride was synthesized using the general procedure shown in Reaction Scheme 6 and Example method 6, Procedure B, above (95% yield), and tert-butyl N-[(1R)-1-[2-(2,6-dioxopiperidin-3-yl)-1-oxo-2,3-dihydro-1H-isoindol-5-yl]ethyl]carbamate (15.0 mg, 0.039 mmol) as a starting material.

LCMS: (ESI+) m/z 287.9 [M+H]⁺

¹H NMR (500 MHz, DMSO) δ 11.00 (s, 1H), 8.44 (s, 3H), 7.82 (dd, J=7.9, 1.4 Hz, 1H), 7.74 (s, 1H), 7.65 (ddd, J=7.9, 3.1, 1.2 Hz, 1H), 5.14 (dd, J=13.3, 5.1 Hz, 1H), 4.61-4.52 (m, 1H), 4.50 (dd, J=17.5, 10.1 Hz, 1H), 4.36 (dd, J=17.5, 9.4 Hz, 1H), 2.93 (ddd, J=17.5, 13.7, 5.4 Hz, 1H), 2.67-2.58 (m, 1H), 2.43 (qd, J=13.3, 4.5 Hz, 1H), 2.06-1.98 (m, 1H), 1.55 (d, J=6.6 Hz, 3H).

### Example 10: Synthesis of N-{[2-(2,6-dioxopiperidin-3-yl)-1,3-dioxo-2,3-dihydro-1H-isoindol-5-yl]methyl}acetamide (12)

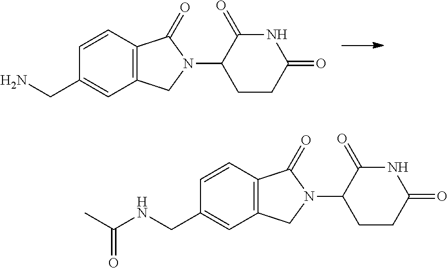 [SMILES: NCc1ccc2c(c1)CN(C1CCC(=O)NC1=O)C2=O ; CC(=O)NCc1ccc2c(c1)CN(C1CCC(=O)NC1=O)C2=O]

3-[5-(aminomethyl)-1-oxo-2,3-dihydro-1H-isoindol-2-yl]piperidine-2,6-dione hydrochloride (20.0 mg, 0.065 mmol) was dissolved in DMF (2.0 mL), and DIPEA (0.034 mL, 0.194 mmol) was added in one portion. Acetyl chloride (7 μL, 0.093 mmol) was added in one portion and the reaction mixture was stirred for 24 h at RT. DMF was removed under reduced pressure and the residue was purified by preparative HPLC to give 9.6 mg of N-{[2-(2,6-dioxopiperidin-3-yl)-1,3-dioxo-2,3-dihydro-1H-isoindol-5-yl]methyl}acetamide (47% yield).

LCMS: (ESI+) m/z 330.0 [M+H]⁺

¹H NMR (500 MHz, DMSO) δ 10.98 (s, 1H), 8.44 (t, J=5.9 Hz, 1H), 7.68 (d, J=7.8 Hz, 1H), 7.48 (s, 1H), 7.40 (d, J=7.8 Hz, 1H), 5.11 (dd, J=13.3, 5.1 Hz, 1H), 4.45 (d, J=17.3 Hz, 1H), 4.37 (d, J=6.0 Hz, 2H), 4.31 (d, J=17.3 Hz, 1H), 2.92 (ddd, J=17.4, 13.7, 5.4 Hz, 1H), 2.64-2.57 (m, 1H), 2.45-2.38 (m, 1H), 2.01 (dtd, J=12.6, 5.3, 2.2 Hz, 1H), 1.90 (s, 3H).

### Example 11: Synthesis of N-{[2-(2,6-dioxopiperidin-3-yl)-6-fluoro-1-oxo-2,3-dihydro-1H-isoindol-5-yl]methyl}acetamide (13)

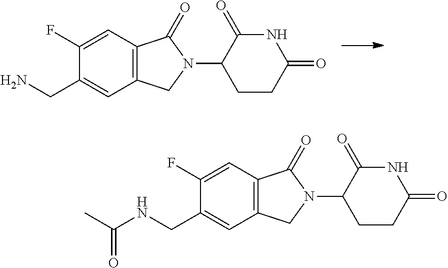 [SMILES: NCc1cc2c(cc1F)C(=O)N(C1CCC(=O)NC1=O)C2 ; CC(=O)NCc1cc2c(cc1F)C(=O)N(C1CCC(=O)NC1=O)C2]

To the mixture of 3-[5-(aminomethyl)-6-fluoro-1-oxo-2,3-dihydro-1H-isoindol-2-yl]piperidine-2,6-dione hydrochloride (10.1 mg, 0.031 mmol), DMAP (0.4 mg, 0.003 mmol), DIPEA (11 μL, 0.062 mmol) and DMF (1.0 mL) was added acetic anhydride (3 μL, 0.031 mmol). Reaction was stirred at RT for 12 h. Upon completion of the reaction, the mixture was evaporated and dry residue was purified by preparative HPLC to give 5.4 mg of N-{[2-(2,6-dioxopiperidin-3-yl)-6-fluoro-1-oxo-2,3-dihydro-1H-isoindol-5-yl]methyl}acetamide (52.1% yield) as a white solid.

LCMS: (ESI+) m/z 334.0 [M+H]⁺, (ESI−) m/z 332.0 [M−H]⁻

¹H NMR (500 MHz, DMSO) δ 10.98 (s, 1H), 8.43 (t, J=5.9 Hz, 1H), 7.54 (d, J=6.3 Hz, 1H), 7.49 (d, J=8.8 Hz, 1H), 5.10 (dd, J=13.3, 5.1 Hz, 1H), 4.43 (d, J=17.1 Hz, 1H), 4.36 (d, J=5.8 Hz, 2H), 4.30 (d, J=17.2 Hz, 1H), 2.91 (ddd, J=17.3, 13.7, 5.5 Hz, 1H), 2.62-2.56 (m, 1H), 2.43-2.37 (m, 1H), 2.01 (dtd, J=12.8, 5.4, 2.3 Hz, 1H), 1.89 (s, 3H).

### Example 12: Synthesis of N-{[2-(2,6-dioxopiperidin-3-yl)-1-oxo-2,3-dihydro-1H-isoindol-5-yl]methyl}-2-ethoxyacetamide (14)

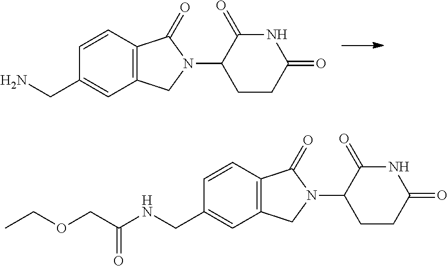 [SMILES: NCc1ccc2c(c1)CN(C1CCC(=O)NC1=O)C2=O ; CCOCC(=O)NCc1ccc2c(c1)CN(C1CCC(=O)NC1=O)C2=O]

3-[5-(aminomethyl)-1-oxo-2,3-dihydro-1H-isoindol-2-yl]piperidine-2,6-dione hydrochloride (20.0 mg, 0.065 mmol) was dissolved in DMF (2.0 mL) and DIPEA (34 μL, 0.194 mmol) was added in one portion. 2-etoxyacetyl chloride (11 IL, 0.097 mmol) was added in one portion and the reaction mixture was stirred for 24 h at RT. DMF was removed under reduced pressure and the residue was purified by preparative HPLC to give 9.9 mg N-{[2-(2,6-dioxopiperidin-3-yl)-1-oxo-2,3-dihydro-1H-isoindol-5-yl]methyl}-2-ethoxyacetamide (42% yield).

LCMS: (ESI+) m/z 360.0 [M+H]⁺

¹H NMR (500 MHz, DMSO) δ 10.98 (s, 1H), 8.39 (t, J=6.2 Hz, 1H), 7.68 (d, J=7.8 Hz, 1H), 7.48 (s, 1H), 7.41 (d, J=7.9 Hz, 1H), 5.11 (dd, J=13.3, 5.1 Hz, 1H), 4.49-4.39 (m, 3H), 4.31 (d, J=17.3 Hz, 1H), 3.91 (s, 2H), 3.52 (q, J=7.0 Hz, 2H), 2.92 (ddd, J=17.4, 13.7, 5.4 Hz, 1H), 2.65-2.57 (m, 1H), 2.40 (qd, J=13.3, 4.5 Hz, 1H), 2.05-1.97 (m, 1H), 1.17 (t, J=7.0 Hz, 3H).

### Example 13: Synthesis of 3-{5-[(butylamino)methyl]-1-oxo-2,3-dihydro-1H-isoindol-2-yl}piperidine-2,6-dione (15)

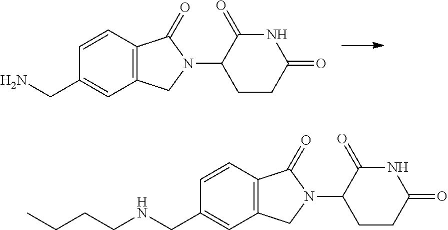 [SMILES: NCc1ccc2c(c1)CN(C1CCC(=O)NC1=O)C2=O ; CCCCNCc1ccc2c(c1)CN(C1CCC(=O)NC1=O)C2=O]

To a solution of 3-[5-(aminomethyl)-1-oxo-2,3-dihydro-1H-isoindol-2-yl]piperidine-2,6-dione hydrochloride (60.0 mg, 0.194 mmol) in DMF (1.0 mL) was added DIPEA (135 μL, 0.775 mmol) followed by iodobutane (24 μL, 0.213 mmol) and the reaction mixture was stirred at RT for 18 h. The solution was concentrated under reduced pressure, the residue was redissolved in small amount of water/DMSO and purified by preparative HPLC to give 2.8 mg 3-{5-[(butylamino)methyl]-1-oxo-2,3-dihydro-1H-isoindol-2-yl}piperidine-2,6-dione formate (3.9% yield).

LCMS: (ESI+) m/z 330.1 [M+H]⁺

¹H NMR (500 MHz, DMSO) δ 10.97 (s, 1H), 8.22 (s, 1H, formic acid), 7.66 (d, J=7.7 Hz, 1H), 7.56 (s, 1H), 7.47 (d, J=7.7 Hz, 1H), 5.10 (dd, J=13.3, 5.1 Hz, 1H), 4.44 (d, J=17.2 Hz, 1H), 4.30 (d, J=17.2 Hz, 1H), 3.82 (s, 2H), 2.96-2.86 (m, 1H), 2.63-2.56 (m, 1H), 2.45-2.37 (m, 1H), 2.00 (ddt, J=10.6, 5.2, 2.7 Hz, 2H), 1.68-1.58 (m, 1H), 1.42 (p, J=7.1 Hz, 2H), 1.35-1.27 (m, 2H), 0.86 (t, J=7.3 Hz, 3H). Exchangeable protons not visible

### Example 14: Synthesis of 3-(5-((dimethylamino)methyl)-1-oxoisoindolin-2-yl)piperidine-2,6-dione (16)

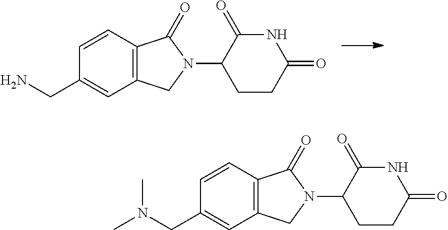 [SMILES: NCc1ccc2c(c1)CN(C1CCC(=O)NC1=O)C2=O ; CN(C)Cc1ccc2c(c1)CN(C1CCC(=O)NC1=O)C2=O]

To a solution of 3-(5-(aminomethyl)-1-oxoisoindolin-2-yl)piperidine-2,6-dione hydrochloride (70 mg, 0.226 mmol) in water/1,4-dioxane (1/1, 2 mL) was added 0.1 mL of 37% formaldehyde (6 eq). The reaction was stirred at RT for 6 h and NaBH₃CN (10 eq) was added. The reaction was then stirred at RT for 2 days and concentrated under reduced pressure. The crude product was purified by preparative TLC to give 13.0 mg of 3-(5-((dimethylamino)methyl)-1-oxoisoindolin-2-yl)piperidine-2,6-dione (19% yield).

LCMS: (ESI+) m/z) 302.0 [M+H]⁺

¹H NMR (500 MHz, DMSO) δ 11.16 (d, J=75.8 Hz, 1H), 11.00 (s, 1H), 7.86 (s, 1H), 7.80 (t, J=8.5 Hz, 1H), 7.75 (d, J=7.9 Hz, 1H), 5.13 (dd, J=13.3, 5.1 Hz, 1H), 4.43 (dt, J=19.7, 17.7 Hz, 4H), 2.97-2.87 (m, 1H), 2.72-2.69 (m, 3H), 2.69 (d, J=1.9 Hz, 3H), 2.60 (d, J=17.5 Hz, 1H), 2.42 (qd, J=13.3, 4.4 Hz, 1H), 2.01 (ddd, J=10.2, 5.1, 3.1 Hz, 1H).

### Example 15: Synthesis of 3-(5-(hydroxymethyl)-1-oxoisoindolin-2-yl)piperidine-2,6-dione (17)

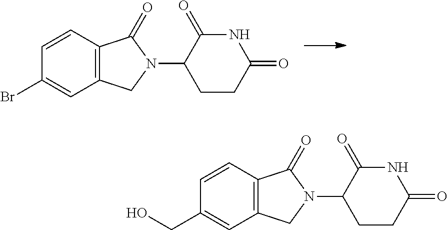 [SMILES: O=C1CCC(N2Cc3cc(Br)ccc3C2=O)C(=O)N1 ; O=C1CCC(N2Cc3cc(CO)ccc3C2=O)C(=O)N1]

3-(5-Bromo-1-oxoisoindolin-2-yl)piperidine-2,6-dione (100 mg, 0.31 mmol) was dissolved in DMF (2 mL). (Tributylstannyl)methanol (149.0 mg, 0.46 mmol, 1.5 eq) and Pd(PPh₃)₄ (35 mg, 0.031 mmol, 0.1 eq) were added and the reaction mixture was stirred at 90° C. for 18 h. The crude mixture was purified by preparative HPLC to give 8 mg of 3-(5-(hydroxymethyl)-1-oxoisoindolin-2-yl)piperidine-2,6-dione (9% yield).

LCMS: (ESI+) m/z 275.2 [M+H]⁺

¹H NMR (400 MHz, DMSO) δ 11.92 (br. s, 1H), 7.67 (d, J=7.8 Hz, 1H), 7.55 (s, 1H), 7.45 (d, J=7.9 Hz, 1H), 5.39 (s, 1H), 5.11 (dd, J=13.4, 5.2 Hz, 1H), 4.62 (d, J=4.5 Hz, 2H), 4.44 (d, J=17.2 Hz, 1H), 4.31 (d, J=17.2 Hz, 1H), 2.91 (td, J=15.6, 13.7, 5.3 Hz, 1H), 2.65-2.55 (m, 1H), 2.46-2.29 (m, 1H), 2.00 (d, J=12.0 Hz, 1H).

### Example 16: Synthesis of {3-[5-(aminomethyl)-1-oxo-2,3-dihydro-1H-isoindol-2-yl]-2,6-dioxopiperidin-1-yl}methyl 2,2-dimethylpropanoate (18)

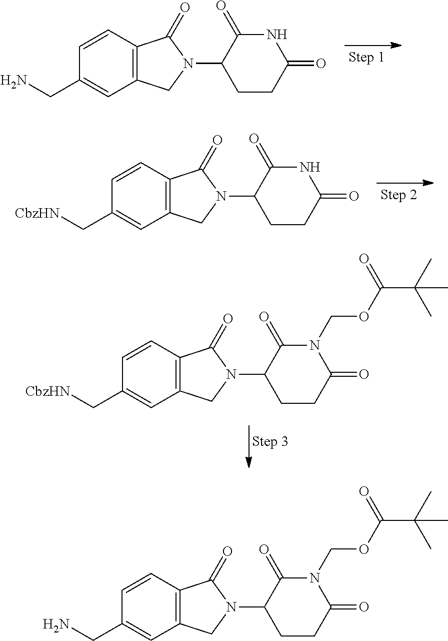 [SMILES: NCc1ccc2c(c1)CN(C1CCC(=O)NC1=O)C2=O ; O=C1CCC(N2Cc3cc(CNC(=O)OCc4ccccc4)ccc3C2=O)C(=O)N1 ; CC(C)(C)C(=O)OCN1C(=O)CCC(N2Cc3cc(CNC(=O)OCc4ccccc4)ccc3C2=O)C1=O ; CC(C)(C)C(=O)OCN1C(=O)CCC(N2Cc3cc(CN)ccc3C2=O)C1=O]

Step 1. To a suspension of 3-[5-(aminomethyl)-1-oxo-2,3-dihydro-1H-isoindol-2-yl]piperidine-2,6-dione hydrochloride (120.0 mg, 0.387 mmol) in ACN (2.0 mL) was added N-(Benzyloxycarbonyloxy)succinimide (101.4 mg, 0.407 mmol), followed by DIPEA (0.169 mL, 0.969 mmol) and the reaction mixture was stirred at RT for 2 h. The solution was concentrated under reduced pressure and the crude product was purified by reversed phase flash column chromatography to give 135.0 mg of benzyl N-{[2-(2,6-dioxopiperidin-3-yl)-1-oxo-2,3-dihydro-1H-isoindol-5-yl]methyl}carbamate (85% yield).

Step 2. In a vial were placed benzyl N-{[2-(2,6-dioxopiperidin-3-yl)-1-oxo-2,3-dihydro-1H-isoindol-5-yl]methyl}carbamate (104.0 mg, 0.255 mmol), Cs₂CO₃ (91.5 mg, 0.281 mmol), tetrabutylammonium iodide (94.3 mg, 0.255 mmol). DMF (2.5 mL) was added followed by chloromethyl pivalate (40 μL, 0.278 mmol) and the reaction mixture was stirred at RT for 18 h. The mixture was filtered through Celite, concentrated under reduced pressure and purified by reversed phase flash column chromatography and flash column chromatography to give 100.0 mg of {3-[5-({[(benzyloxy)carbonyl]amino}methyl)-1-oxo-2,3-dihydro-1H-isoindol-2-yl]-2,6-dioxopiperidin-1-yl}methyl 2,2-dimethylpropanoate (75% yield).

Step 3. To a solution of {3-[5-({[(benzyloxy)carbonyl]amino}methyl)-1-oxo-2,3-dihydro-1H-isoindol-2-yl]-2,6-dioxopiperidin-1-yl}methyl 2,2-dimethylpropanoate (100.0 mg, 0.192 mmol) in ethanol (10.0 mL) was added Pd/C (10.0 mg, 10% wt) and the reaction mixture was stirred under hydrogen atmosphere (1 bar) for 1 h. The reaction mixture was filtered, concentrated under reduced pressure and purified by preparative HPLC to give 36.0 mg of {3-[5-(aminomethyl)-1-oxo-2,3-dihydro-1H-isoindol-2-yl]-2,6-dioxopiperidin-1-yl}methyl 2,2-dimethylpropanoate (48% yield).

LCMS: (ESI+) m/z 388.1 [M+H]⁺

¹H NMR (500 MHz, DMSO) δ 8.27 (s, 2H), 7.75 (d, J=7.8 Hz, 1H), 7.65 (s, 1H), 7.56 (d, J=7.8 Hz, 1H), 5.66 (d, J=9.6 Hz, 1H), 5.62 (d, J=9.5 Hz, 1H), 5.32 (dd, J=13.4, 5.1 Hz, 1H), 4.51 (d, J=17.2 Hz, 1H), 4.30 (d, J=17.2 Hz, 1H), 4.01 (s, 2H), 3.14 (ddd, J=17.6, 13.7, 5.4 Hz, 1H), 2.86 (ddd, J=17.5, 4.1, 2.3 Hz, 1H), 2.44 (qd, J=13.4, 4.3 Hz, 2H), 2.10 (dtt, J=9.0, 5.2, 2.4 Hz, 1H), 1.13 (s, 9H).

### Example 17: Synthesis of [2-({[2-(2,6-dioxopiperidin-3-yl)-1-oxo-2,3-dihydro-1H-isoindol-5-yl]methyl}carbamoyl)phenyl]methyl acetate (19)

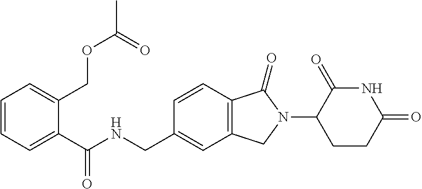 [SMILES: CC(=O)OCc1ccccc1C(=O)NCc1ccc2c(c1)CN(C1CCC(=O)NC1=O)C2=O]

[2-({[2-(2,6-dioxopiperidin-3-yl)-1-oxo-2,3-dihydro-1H-isoindol-5-yl]methyl}carbamoyl)phenyl]methyl acetate was synthesized using the general procedure shown in Reaction Scheme 1 and Example method 1, above (59% yield), and 2-(acetoxymethyl)benzoic acid (30.0 mg, 0.0.154 mmol), 3-[5-(aminomethyl)-1-oxo-2,3-dihydro-1H-isoindol-2-yl]piperidine-2,6-dione hydrochloride (1.0 eq) as a starting materials.

LCMS: (ESI+) m/z 450.15 [M+H]⁺

¹H NMR (500 MHz, DMSO) δ 10.90 (s, 1H), 8.98 (t, J=6.0 Hz, 1H), 7.63 (d, J=7.8 Hz, 1H), 7.50 (s, 1H), 7.51-7.46 (m, 1H), 7.45-7.37 (m, 3H), 7.35 (td, J=7.2, 2.1 Hz, 1H), 5.17 (s, 2H), 5.04 (dd, J=13.4, 5.1 Hz, 1H), 4.49 (d, J=6.0 Hz, 2H), 4.38 (d, J=17.3 Hz, 1H), 4.26 (d, J=17.3 Hz, 1H), 2.85 (ddd, J=17.4, 13.7, 5.4 Hz, 1H), 2.53 (ddd, J=16.9, 4.1 2.1 Hz, 1H), 2.33 (qd, J=13.2, 4.3 Hz, 1H), 1.97-1.93 (m, 1H), 1.92 (s, 3H).

### Example 18: Synthesis of N-{[2-(2,6-dioxopiperidin-3-yl)-1-oxo-2,3-dihydro-1H-isoindol-5-yl]methyl}-2-(hydroxymethyl)benzamide (20)

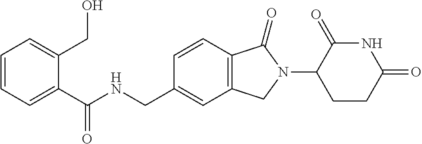 [SMILES: O=C1CCC(N2Cc3cc(CNC(=O)c4ccccc4CO)ccc3C2=O)C(=O)N1]

N-{[2-(2,6-dioxopiperidin-3-yl)-1-oxo-2,3-dihydro-1H-isoindol-5-yl]methyl}-2-(hydroxymethyl)benzamide was synthesized using the general procedure shown in Reaction Scheme 1 and Example method 1, above (3.4% yield), and 2-(Hydroxymethyl)benzoic acid (49.1 mg, 0.323 mmol), 3-[5-(aminomethyl)-1-oxo-2,3-dihydro-1H-isoindol-2-yl]piperidine-2,6-dione hydrochloride (1.0 eq) as a starting materials.

LCMS: (ESI+) m/z 408.2 [M+H]⁺

¹H NMR (500 MHz, DMSO) δ 10.99 (s, 1H), 9.02 (t, J=6.0 Hz, 1H), 7.72 (d, J=7.8 Hz, 1H), 7.59 (s, 1H), 7.60-7.55 (m, 1H), 7.54-7.49 (m, 2H), 7.48 (td, J=7.5, 1.3 Hz, 1H), 7.35 (td, J=7.5, 1.2 Hz, 1H), 5.24 (t, J=5.6 Hz, 1H), 5.13 (dd, J=13.3, 5.1 Hz, 1H), 4.63 (d, J=5.5 Hz, 2H), 4.58 (d, J=6.0 Hz, 2H), 4.48 (d, J=17.4 Hz, 1H), 4.35 (d, J=17.3 Hz, 1H), 2.93 (ddd, J=17.4, 13.6, 5.4 Hz, 1H), 2.62 (ddd, J=17.1, 4.3, 2.3 Hz, 1H), 2.41 (qd, J=13.3, 4.5 Hz, 1H), 2.07-1.99 (m, 1H).

### Example 19: Synthesis of 2-[1-({[2-(2,6-dioxopiperidin-3-yl)-1-oxo-2,3-dihydro-1H-isoindol-5-yl]methyl}carbamoyl)-2-methylpropan-2-yl]-3,5-dimethyl phenyl acetate (21)

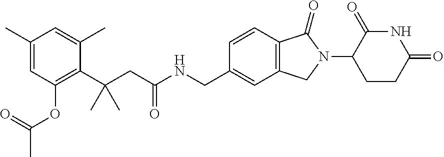 [SMILES: CC(=O)Oc1cc(C)cc(C)c1C(C)(C)CC(=O)NCc1ccc2c(c1)CN(C1CCC(=O)NC1=O)C2=O]

2-[1-({[2-(2,6-dioxopiperidin-3-yl)-1-oxo-2,3-dihydro-1H-isoindol-5-yl]methyl}carbamoyl)-2-methylpropan-2-yl]-3,5-dimethylphenyl acetate was synthesized using the general procedure shown in Reaction Scheme 1 and Example method 1, above (80% yield), and 3-(2-Acetoxy-4,6-dimethylphenyl)-3-methylbutyric acid (30.0 mg, 0.113 mmol), 3-[5-(aminomethyl)-1-oxo-2,3-dihydro-1H-isoindol-2-yl]piperidine-2,6-dione hydrochloride (1.2 eq) as a starting materials.

LCMS: (ESI+) m/z 520.0 [M+H]⁺

¹H NMR (500 MHz, DMSO) δ 10.97 (s, 1H), 8.14 (t, J=6.0 Hz, 1H), 7.60 (d, J=7.8 Hz, 1H), 7.28 (s, 1H), 7.22 (d, J=7.5 Hz, 1H), 6.80-6.77 (m, 1H), 6.62-6.59 (m, 1H), 5.10 (dd, J=13.3, 5.1 Hz, 1H), 4.39 (d, J=17.2 Hz, 1H), 4.31-4.23 (m, 3H), 2.91 (ddd, J=17.3, 13.7, 5.4 Hz, 1H), 2.64-2.51 (m, 3H), 2.47 (s, 3H), 2.45-2.35 (m, 1H), 2.26 (s, 3H), 2.17 (s, 3H), 2.00 (ddq, J=10.4, 5.3, 3.1, 2.6 Hz, 1H), 1.49 (s, 6H).

### Example 20: Synthesis of 4-(((((2-(2,6-dioxopiperidin-3-yl)-1-oxoisoindolin-5-yl)methyl)carbamoyl)oxy)methyl)phenyl acetate (22)

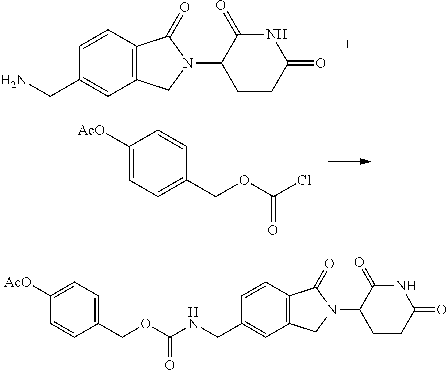

To the solution of 3-(5-(aminomethyl)-1-oxoisoindolin-2-yl)piperidine-2,6-dione hydrochloride (25 mg, 0.081 mmol) and DIPEA (3 eq) in DMF (1 mL) was added 4-(acetoxy)benzyl chloroformate (28 mg, 0.121 mmol) and the reaction mixture was stirred at RT for 18 h. The solvent was removed in vacuo and the product was purified by preparative HPLC to give 11.0 mg of 4-(((((2-(2,6-dioxopiperidin-3-yl)-1-oxoisoindolin-5-yl)methyl)carbamoyl)oxy)methyl)phenyl acetate (28% yield).

LCMS: (ESI+) m/z 466.1 [M+H]⁺

¹H NMR (500 MHz, DMSO) δ 10.98 (s, 1H), 7.95 (t, J=6.2 Hz, 1H), 7.69 (d, J=7.7 Hz, 1H), 7.46 (s, 1H), 7.44-7.38 (m, 3H), 7.13 (d, J=8.4 Hz, 2H), 5.11 (dd, J=13.3, 5.1 Hz, 1H), 5.06 (s, 2H), 4.44 (d, J=17.3 Hz, 1H), 4.36-4.27 (m, 3H), 2.92 (ddd, J=17.5, 13.7, 5.5 Hz, 1H), 2.65-2.57 (m, 1H), 2.40 (qd, J=13.2, 4.5 Hz, 1H), 2.27 (s, 3H), 2.01 (ddq, J=10.2, 5.1, 2.8, 2.1 Hz, 1H)

### Example 21: Synthesis of 1-((((2-(2,6-dioxopiperidin-3-yl)-1-oxoisoindolin-5-yl)methyl)carbamoyl)oxy)ethyl isobutyrate (23)

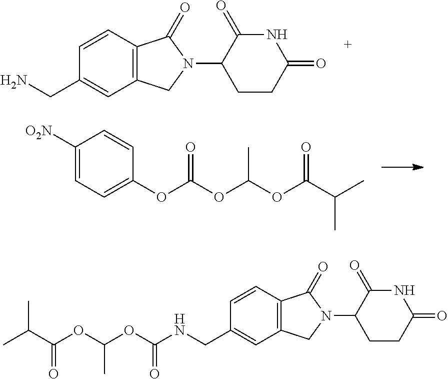

To a solution of 3-(5-(aminomethyl)-1-oxoisoindolin-2-yl)piperidine-2,6-dione hydrochloride (25 mg, 0.081 mmol) and 1-(((4-nitrophenoxy)carbonyl)oxy)ethyl isobutyrate (26.4 mg, 0.089 mmol) in DMF (1 mL) was added DIPEA (0.028 mL, 0.161 mmol) and the reaction mixture was stirred at RT for 18 h. The product was purified by preparative HPLC to give 15.8 mg of 1-((((2-(2,6-dioxopiperidin-3-yl)-1-oxoisoindolin-5-yl)methyl)carbamoyl)oxy)ethyl isobutyrate (45.4% yield).

LCMS: (ESI+) m/z) 430.1 [M−H]⁻

¹H NMR (500 MHz, DMSO) δ 10.98 (s, 1H), 8.17-8.07 (m, 1H), 7.69 (d, J=7.8 Hz, 1H), 7.47 (s, 1H), 7.40 (d, J=7.8 Hz, 1H), 6.69 (q, J=5.4 Hz, 1H), 5.11 (dd, J=13.3, 5.1 Hz, 1H), 4.44 (dd, J=17.4, 2.2 Hz, 1H), 4.37-4.25 (m, 3H), 2.92 (ddd, J=17.4, 13.7, 5.4 Hz, 1H), 2.66-2.56 (m, 1H), 2.51 (hept, J=7.0 Hz, 1H), 2.40 (qd, J=13.4, 4.6 Hz, 1H), 2.01 (ddq, J=10.4, 5.2, 3.1, 2.7 Hz, 1H), 1.42 (d, J=5.4 Hz, 3H), 1.10-1.03 (m, 6H).

### Example 22: Synthesis of (5-methyl-2-oxo-2H-1,3-dioxol-4-yl)methyl N-{[2-(2,6-dioxopiperidin-3-yl)-1-oxo-2,3-dihydro-1H-isoindol-5-yl]methyl}carbamate (24)

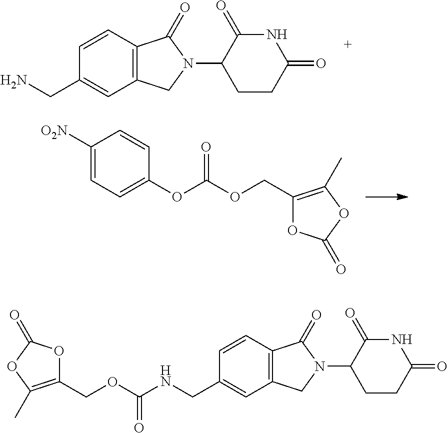

In a 10 mL vial was placed 3-[5-(aminomethyl)-1-oxo-2,3-dihydro-1H-isoindol-2-yl]piperidine-2,6-dione hydrochloride (20.0 mg, 0.065 mmol), (5-methyl-2-oxo-1,3-dioxol-4-yl)methyl 4-nitrophenyl carbonate (21.0 mg, 0.071 mmol) and DMF (1 mL). DIPEA (0.022 mL, 0.129 mmol) was added and the reaction mixture was stirred at RT for 18 h. Solvent was removed under reduced pressure and the residue was purified by preparative HPLC to give 21.4 mg of (5-methyl-2-oxo-2H-1,3-dioxol-4-yl)methyl N-{[2-(2,6-dioxopiperidin-3-yl)-1-oxo-2,3-dihydro-1H-isoindol-5-yl]methyl}carbamate (77% yield).

LCMS: (ESI+) m/z 430.3 [M+H]⁺

¹H NMR (500 MHz, DMSO) δ 10.98 (s, 1H), 8.05 (t, J=6.1 Hz, 1H), 7.69 (d, J=7.8 Hz, 1H), 7.48 (s, 1H), 7.41 (d, J=7.9 Hz, 1H), 5.11 (dd, J=13.3, 5.1 Hz, 1H), 4.91 (s, 2H), 4.45 (d, J=17.3 Hz, 1H), 4.36-4.23 (m, 3H), 2.92 (ddd, J=17.4, 13.7, 5.4 Hz, 1H), 2.66-2.57 (m, 1H), 2.40 (ddd, J=17.8, 12.8, 4.1 Hz, 1H), 2.16 (s, 3H), 2.01 (dtd, J=12.6, 5.2, 2.2 Hz, 1H).

### Example 23: Synthesis of tert-butyl (2-(((2-(2,6-dioxopiperidin-3-yl)-1-oxoisoindolin-5-yl)methyl)amino)-2-oxoethyl)carbamate (26) and 2-amino-N-((2-(2,6-dioxopiperidin-3-yl)-1-oxoisoindolin-5-yl)methyl)acetamide (25)

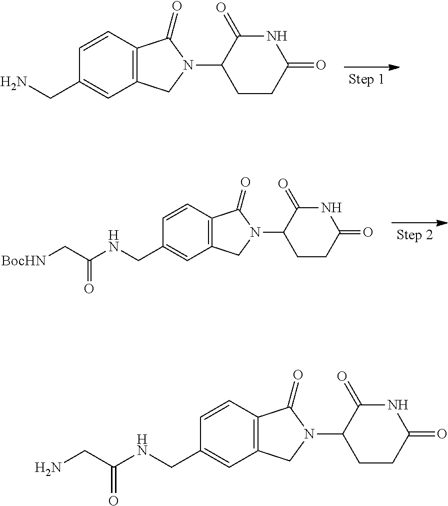 [SMILES: NCc1ccc2c(c1)CN(C1CCC(=O)NC1=O)C2=O ; CC(C)(C)OC(=O)NCC(=O)NCc1ccc2c(c1)CN(C1CCC(=O)NC1=O)C2=O ; NCC(=O)NCc1ccc2c(c1)CN(C1CCC(=O)NC1=O)C2=O]

Step 1. tert-butyl (2-(((2-(2,6-dioxopiperidin-3-yl)-1-oxoisoindolin-5-yl)methyl)amino)-2-oxoethyl)carbamate was synthesized using the general procedure shown in Reaction Scheme 1 and Example method 1, above (86% yield), and 3-(5-(aminomethyl)-1-oxoisoindolin-2-yl)piperidine-2,6-dione hydrochloride (60 mg, 0.194 mmol) and (tert-butoxycarbonyl)glycine (1.200 eq) as starting materials.

LCMS: (ESI+) m/z 331.2 [M+H—BOC]⁺; m/z 429.3 [M−H]⁻

¹H NMR (500 MHz, DMSO) δ 10.97 (s, 1H), 8.39 (t, J=6.0 Hz, 1H), 7.66 (d, J=7.8 Hz, 1H), 7.47 (s, 1H), 7.40 (d, J=7.8 Hz, 1H), 7.00 (t, J=5.9 Hz, 1H), 5.10 (dd, J=13.3, 5.1 Hz, 1H), 4.47-4.23 (m, 4H), 3.59 (d, J=6.0 Hz, 2H), 2.91 (ddd, J=17.4, 13.7, 5.4 Hz, 1H), 2.63-2.56 (m, 1H), 2.45-2.34 (m, 1H), 2.04-1.94 (m, 1H), 1.39 (s, 9H).

Step 2. 2-amino-N-((2-(2,6-dioxopiperidin-3-yl)-1-oxoisoindolin-5-yl)methyl)acetamide was synthesized using the general procedure shown in Reaction Scheme 6 and Example method 6, procedure A, above (31% yield), and tert-butyl (2-(((2-(2,6-dioxopiperidin-3-yl)-1-oxoisoindolin-5-yl)methyl)amino)-2-oxoethyl)carbamate (51.7 mg, 0.120 mmol) as starting material.

LCMS: (ESI+) m/z 331.3 [M+H]⁺

¹H NMR (500 MHz, DMSO) δ 10.95 (s, 1H), 8.91 (t, J=5.8 Hz, 1H), 7.72 (dd, J=22.7, 7.8 Hz, 1H), 7.52 (s, 1H), 7.45 (t, J=12.5 Hz, 1H), 5.11 (dd, J=13.3, 5.1 Hz, 1H), 4.53-4.20 (m, 4H), 3.60 (s, 2H), 2.91 (ddd, J=17.4, 13.7, 5.4 Hz, 1H), 2.61 (ddd, J=17.2, 4.2, 2.2 Hz, 1H), 2.40 (qd, J=13.3, 4.5 Hz, 1H), 2.01 (dtd, J=12.5, 5.2, 2.2 Hz, 1H).

### Example 24: Synthesis of tert-butyl ((S)-1-(((2-((S)-2,6-dioxopiperidin-3-yl)-1-oxoisoindolin-5-yl)methyl)amino)-3-(1H-imidazol-4-yl)-1-oxopropan-2-yl)carbamate (30), tert-butyl ((S)-1-(((2-((R)-2,6-dioxopiperidin-3-yl)-1-oxoisoindolin-5-yl)methyl)amino)-3-(1H-imidazol-4-yl)-1-oxopropan-2-yl)carbamate (29), (S)-2-amino-N-((2-((R)-2,6-dioxopiperidin-3-yl)-1-oxoisoindolin-5-yl)methyl)-3-(1H-imidazol-4-yl)propanamide (28) and (S)-2-amino-N-((2-((R)-2,6-dioxopiperidin-3-yl)-1-oxoisoindolin-5-yl)methyl)-3-(1H-imidazol-4-yl)propanamide (27)

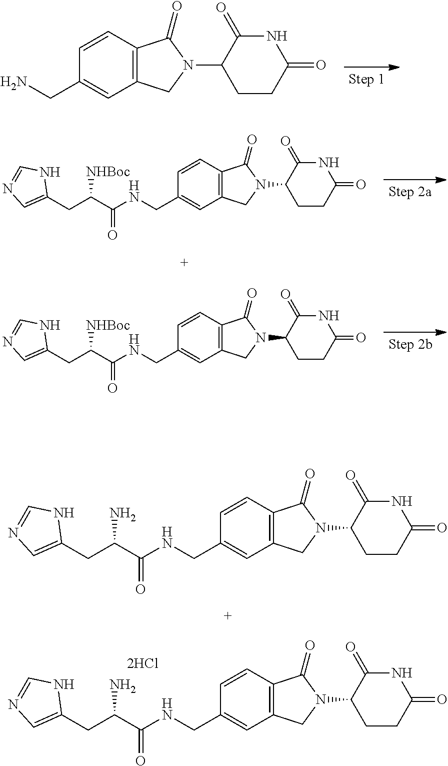

Step 1. 3-[5-(aminomethyl)-1-oxo-2,3-dihydro-1H-isoindol-2-yl]piperidine-2,6-dione hydrochloride (60.0 mg, 0.194 mmol) and Boc-His-OH (59.3 mg, 0.232 mmol) were dissolved in DMF (6 mL). DIPEA (0.074 mL, 0.426 mmol) was added followed by HATU (88.4 mg, 0.232 mmol) and the resulting solution was stirred at RT for 18 h. The solvent was removed under reduced pressure and the residue was purified by preparative HPLC to give 41 mg of tert-butyl ((S)-1-(((2-((S)-2,6-dioxopiperidin-3-yl)-1-oxoisoindolin-5-yl)methyl)amino)-3-(1H-imidazol-4-yl)-1-oxopropan-2-yl)carbamate (41% yield) of and 21.0 mg of tert-butyl ((S)-1-(((2-((R)-2,6-dioxopiperidin-3-yl)-1-oxoisoindolin-5-yl)methyl)amino)-3-(1H-imidazol-4-yl)-1-oxopropan-2-yl)carbamate (21% yield).

### tert-butyl ((S)-1-(((2-((S)-2,6-dioxopiperidin-3-yl)-1-oxoisoindolin-5-yl)methyl)amino)-3-(1H-imidazol-4-yl)-1-oxopropan-2-yl)carbamate

LCMS: (ESI+) m/z 511.6 [M+H]⁺

¹H NMR (500 MHz, DMSO) δ 13.99 (s, 2H), 10.98 (s, 1H), 8.92 (s, 1H), 8.53 (t, J=5.8 Hz, 1H), 7.66 (d, J=7.8 Hz, 1H), 7.42 (s, 1H), 7.32 (d, J=8.3 Hz, 2H), 7.22 (d, J=8.4 Hz, 1H), 5.12 (dd, J=13.3, 5.1 Hz, 1H), 4.47-4.24 (m, 5H), 3.11 (dd, J=15.0, 5.8 Hz, 1H), 2.98-2.86 (m, 2H), 2.67-2.56 (m, 1H), 2.46-2.34 (m, 1H), 2.05-1.97 (m, 1H), 1.36 (s, 9H).

### tert-butyl ((S)-1-(((2-((R)-2,6-dioxopiperidin-3-yl)-1-oxoisoindolin-5-yl)methyl)amino)-3-(1H-imidazol-4-yl)-1-oxopropan-2-yl)carbamate

LCMS: (ESI+) m/z 511.6 [M+H]⁺

¹H NMR (500 MHz, DMSO) δ 11.86 (s, 1H), 10.98 (s, 1H), 8.40 (t, J=5.7 Hz, 1H), 7.69-7.49 (m, 2H), 7.24 (s, 2H), 7.06 (d, J=7.8 Hz, 1H), 6.81 (s, 1H), 5.11 (dd, J=13.3, 5.1 Hz, 1H), 4.45-4.17 (m, 5H), 2.99-2.78 (m, 3H), 2.62 (t, J=12.2 Hz, 1H), 2.48-2.35 (m, 1H), 2.01 (ddd, J=10.4, 5.2, 2.7 Hz, 1H), 1.39 (s, 9H).

Step 2a: (S)-2-amino-N-((2-((S)-2,6-dioxopiperidin-3-yl)-1-oxoisoindolin-5-yl)methyl)-3-(1H-imidazol-4-yl)propanamide was synthesized using the general procedure shown in Reaction Scheme 6 and Example method 6, procedure B, above (100% yield), and tert-butyl ((S)-1-(((2-((S)-2,6-dioxopiperidin-3-yl)-1-oxoisoindolin-5-yl)methyl)amino)-3-(1H-imidazol-4-yl)-1-oxopropan-2-yl)carbamate (10.0 mg, 0.020 mmol) as starting material.

LCMS: (ESI+) m/z 411.0 [M+H]⁺

¹H NMR (500 MHz, DMSO) δ 14.58 (s, 1H), 14.26 (s, 1H), 10.99 (s, 1H), 9.25 (dd, J=13.1, 6.1 Hz, 1H), 9.06 (s, 1H), 8.52 (s, 3H), 7.70 (d, J=7.8 Hz, 1H), 7.51 (s, 1H), 7.42 (s, 1H), 7.29 (d, J=7.9 Hz, 1H), 5.12 (dd, J=13.3, 5.1 Hz, 1H), 4.52-4.37 (m, 3H), 3.75-3.65 (m, 1H), 3.53-3.45 (m, 1H), 3.30-3.17 (m, 2H), 2.94 (ddd, J=17.5, 13.7, 5.4 Hz, 1H), 2.66-2.57 (m, 1H), 2.47-2.35 (m, 1H), 2.02 (dtd, J=12.4, 5.1, 2.1 Hz, 1H).

Step 2b: (S)-2-amino-N-((2-((R)-2,6-dioxopiperidin-3-yl)-1-oxoisoindolin-5-yl)methyl)-3-(1H-imidazol-4-yl)propanamide was synthesized using the general procedure shown in Reaction Scheme 6 and Example method 6, procedure B, above (100% yield), and tert-butyl ((S)-1-(((2-((R)-2,6-dioxopiperidin-3-yl)-1-oxoisoindolin-5-yl)methyl)amino)-3-(1H-imidazol-4-yl)-1-oxopropan-2-yl)carbamate (10.0 mg, 0.020 mmol) as starting material.

LCMS: (ESI+) m/z 411.2 [M+H]⁺

¹H NMR (500 MHz, DMSO) δ 14.71 (s, 1H), 14.39 (s, 1H), 10.99 (s, 1H), 9.35 (dd, J=13.5, 6.1 Hz, 1H), 9.08 (d, J=0.8 Hz, 1H), 8.60 (s, 3H), 7.69 (d, J=7.8 Hz, 1H), 7.53 (s, 1H), 7.42 (s, 1H), 7.31 (d, J=8.0 Hz, 1H), 5.12 (dd, J=13.3, 5.1 Hz, 1H), 4.53-4.37 (m, 3H), 3.77-3.63 (m, 1H), 3.53-3.44 (m, 1H), 3.35-3.19 (m, 2H), 2.93 (ddd, J=17.4, 13.7, 5.4 Hz, 1H), 2.69-2.56 (m, 1H), 2.47-2.33 (m, 1H), 2.02 (ddd, J=10.2, 5.2, 3.1 Hz, 1H).

### Example 25: Synthesis of tert-butyl (1-(((2-(2,6-dioxopiperidin-3-yl)-1-oxoisoindolin-5-yl)methyl)amino)-3-methyl-1-oxobutan-2-yl)carbamate (31) and 2-amino-N-((2-(2,6-dioxopiperidin-3-yl)-1-oxoisoindolin-5-yl)methyl)-3-methylbutanamide (32)

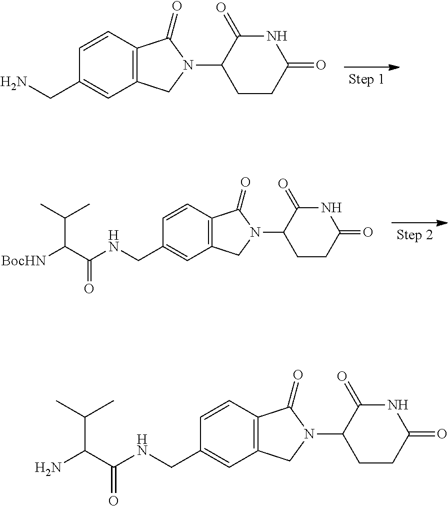 [SMILES: NCc1ccc2c(c1)CN(C1CCC(=O)NC1=O)C2=O ; CC(C)C(NC(=O)OC(C)(C)C)C(=O)NCc1ccc2c(c1)CN(C1CCC(=O)NC1=O)C2=O ; CC(C)C(N)C(=O)NCc1ccc2c(c1)CN(C1CCC(=O)NC1=O)C2=O]

Step 1. tert-butyl (1-(((2-(2,6-dioxopiperidin-3-yl)-1-oxoisoindolin-5-yl)methyl)amino)-3-methyl-1-oxobutan-2-yl)carbamate was synthesized using the general procedure shown in Reaction Scheme 1 and Example method 1, above (76% yield), and 3-(5-(aminomethyl)-1-oxoisoindolin-2-yl)piperidine-2,6-dione hydrochloride (50 mg, 0.161 mmol) and (tert-butoxycarbonyl)valine (1.200 eq) as starting materials.

LCMS: (ESI+) m/z 471.6 [M−H]⁻

¹H NMR (500 MHz, DMSO) δ 10.96 (s, 1H), 8.46 (t, J=5.7 Hz, 1H), 7.65 (d, J=7.8 Hz, 1H), 7.47 (s, 1H), 7.41 (d, J=7.8 Hz, 1H), 6.74 (d, J=8.7 Hz, 1H), 5.10 (dd, J=13.3, 5.1 Hz, 1H), 4.51-4.21 (m, 4H), 3.78 (t, J=7.9 Hz, 1H), 2.91 (ddd, J=17.4, 13.7, 5.4 Hz, 1H), 2.67-2.55 (m, 1H), 2.43-2.34 (m, 1H), 2.04-1.90 (m, 2H), 1.38 (d, J=7.4 Hz, 9H), 0.84 (s, 3H), 0.83 (s, 3H)

Step 2. 2-amino-N-((2-(2,6-dioxopiperidin-3-yl)-1-oxoisoindolin-5-yl)methyl)-3-methylbutanamide was synthesized using the general procedure shown in Reaction Scheme 6 and Example method 6, procedure A, above (92% yield), and tert-butyl (1-(((2-(2,6-dioxopiperidin-3-yl)-1-oxoisoindolin-5-yl)methyl)amino)-3-methyl-1-oxobutan-2-yl)carbamate (88.8 mg, 0.188 mmol) as starting material.

LCMS: (ESI+) m/z 372.8 [M+H]⁺

¹H NMR (500 MHz, DMSO) δ 10.98 (s, 1H), 8.90 (s, 1H), 7.71 (d, J=7.8 Hz, 1H), 7.52 (s, 1H), 7.45 (d, J=7.8 Hz, 1H), 5.11 (dd, J=13.3, 5.1 Hz, 1H), 4.53-4.24 (m, 4H), 3.53 (d, J=4.5 Hz, 1H), 3.01-2.84 (m, 1H), 2.67-2.56 (m, 1H), 2.45-2.34 (m, 1H), 2.12-2.04 (m, 1H), 2.04-1.96 (m, 1H), 0.93 (d, J=6.9 Hz, 3H), 0.91 (d, J=6.9 Hz, 3H)

### Example 26: Synthesis of tert-butyl (1-(((2-(2,6-dioxopiperidin-3-yl)-1-oxoisoindolin-5-yl)methyl)amino)-2-methyl-1-oxopropan-2-yl)carbamate (33) and 2-amino-N-((2-(2,6-dioxopiperidin-3-yl)-1-oxoisoindolin-5-yl)methyl)-2-methylpropanamide (34)

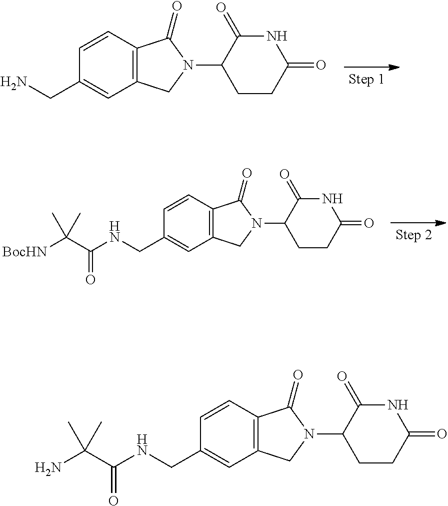 [SMILES: NCc1ccc2c(c1)CN(C1CCC(=O)NC1=O)C2=O ; CC(C)(C)OC(=O)NC(C)(C)C(=O)NCc1ccc2c(c1)CN(C1CCC(=O)NC1=O)C2=O ; CC(C)(N)C(=O)NCc1ccc2c(c1)CN(C1CCC(=O)NC1=O)C2=O]

Step 1. tert-butyl (1-(((2-(2,6-dioxopiperidin-3-yl)-1-oxoisoindolin-5-yl)methyl)amino)-2-methyl-1-oxopropan-2-yl)carbamate was synthesized using the general procedure shown in Reaction Scheme 1 and Example method 1, above (67% yield), and 3-(5-(aminomethyl)-1-oxoisoindolin-2-yl)piperidine-2,6-dione hydrochloride (30 mg, 0.097 mmol) and 2-((tert-butoxycarbonyl)amino)-2-methylpropanoic acid (1.200 eq) as starting materials.

LCMS: (ESI+) m/z 358.8 [M+H—BOC]⁺; (ESI−) m/z 457.0 [M−H]⁻

¹H NMR (500 MHz, DMSO) δ 10.96 (s, 1H), 8.19 (s, 1H), 7.61 (d, J=7.8 Hz, 1H), 7.49 (s, 1H), 7.41 (d, J=7.9 Hz, 1H), 6.92 (s, 1H), 5.10 (dd, J=13.3, 5.1 Hz, 1H), 4.34 (dt, J=53.9, 17.2 Hz, 4H), 2.91 (ddd, J=17.4, 13.7, 5.4 Hz, 1H), 2.59 (dd, J=14.5, 2.0 Hz, 1H), 2.44-2.37 (m, 1H), 1.99 (ddd, J=10.4, 5.3, 3.1 Hz, 1H), 1.37 (d, J=4.2 Hz, 6H), 1.31 (t, J=9.6 Hz, 9H).

Step 2. 2-amino-N-((2-(2,6-dioxopiperidin-3-yl)-1-oxoisoindolin-5-yl)methyl)-2-methylpropanamide was synthesized using the general procedure shown in Reaction Scheme 6 and Example method 6, procedure A, above (83% yield), and tert-butyl (1-(((2-(2,6-dioxopiperidin-3-yl)-1-oxoisoindolin-5-yl)methyl)amino)-2-methyl-1-oxopropan-2-yl)carbamate (26.1 mg, 0.057 mmol) as starting material.

LCMS: (ESI+) m/z 358.9

¹H NMR (500 MHz, DMSO) δ 10.98 (s, 1H), 8.96 (t, J=5.9 Hz, 1H), 7.70 (d, J=7.8 Hz, 1H), 7.48 (s, 1H), 7.40 (d, J=7.9 Hz, 1H), 5.10 (dd, J=13.3, 5.1 Hz, 1H), 4.49-4.26 (m, 4H), 2.91 (ddd, J=17.4, 13.7, 5.4 Hz, 1H), 2.65-2.57 (m, 1H), 2.45-2.34 (m, 1H), 2.01 (dtd, J=12.5, 5.2, 2.1 Hz, 1H), 1.50 (d, J=1.5 Hz, 6H)

### Example 27: Synthesis of tert-butyl (1-(((2-(2,6-dioxopiperidin-3-yl)-1-oxoisoindolin-5-yl)methyl)amino)-3-(1H-indol-3-yl)-1-oxopropan-2-yl)carbamate (35) and 2-amino-N-((2-(2,6-dioxopiperidin-3-yl)-1-oxoisoindolin-5-yl)methyl)-3-(1H-indol-3-yl)propanamide (37)

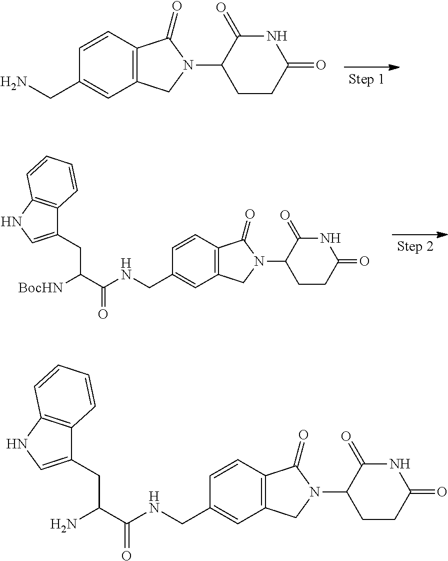 [SMILES: NCc1ccc2c(c1)CN(C1CCC(=O)NC1=O)C2=O ; CC(C)(C)OC(=O)NC(Cc1c[nH]c2ccccc12)C(=O)NCc1ccc2c(c1)CN(C1CCC(=O)NC1=O)C2=O ; NC(Cc1c[nH]c2ccccc12)C(=O)NCc1ccc2c(c1)CN(C1CCC(=O)NC1=O)C2=O]

Step 1. tert-butyl (1-(((2-(2,6-dioxopiperidin-3-yl)-1-oxoisoindolin-5-yl)methyl)amino)-3-(1H-indol-3-yl)-1-oxopropan-2-yl)carbamate was synthesized using the general procedure shown in Reaction Scheme 1 and Example method 1, above (66% yield), and 3-(5-(aminomethyl)-1-oxoisoindolin-2-yl)piperidine-2,6-dione hydrochloride (50 mg, 0.161 mmol) and (tert-butoxycarbonyl)tryptophan (1.200 eq) as starting materials.

LCMS: (ESI+) m/z 460.3 [M+H—BOC]⁺; (ESI−) m/z 558.4 [M−H]⁻

¹H NMR (500 MHz, DMSO) δ 10.97 (s, 1H), 10.82 (s, 1H), 8.49 (t, J=5.8 Hz, 1H), 7.67-7.54 (m, 2H), 7.34 (d, J=8.1 Hz, 1H), 7.28 (d, J=6.1 Hz, 2H), 7.15 (s, 1H), 7.11-7.02 (m, 1H), 6.97 (t, J=7.4 Hz, 1H), 6.87 (d, J=8.1 Hz, 1H), 5.10 (dd, J=13.3, 5.0 Hz, 1H), 4.45-4.12 (m, 5H), 3.11 (dd, J=14.4, 5.8 Hz, 1H), 2.92 (ddd, J=19.0, 13.0, 6.6 Hz, 2H), 2.65-2.56 (m, 1H), 2.44-2.34 (m, 1H), 2.04-1.95 (m, 1H), 1.34 (s, 9H)

Step 2. 2-amino-N-((2-(2,6-dioxopiperidin-3-yl)-1-oxoisoindolin-5-yl)methyl)-3-(1H-indol-3-yl)propanamide was synthesized using the general procedure shown in Reaction Scheme 6 and Example method 6, procedure A, above (63.3% yield), and tert-butyl (1-(((2-(2,6-dioxopiperidin-3-yl)-1-oxoisoindolin-5-yl)methyl)amino)-3-(1H-indol-3-yl)-1-oxopropan-2-yl)carbamate (15.0 mg, 0.027 mmol) as starting material.

LCMS: (ESI+) m/z 460.2 [M+H]⁺

¹H NMR (500 MHz, DMSO) δ 10.98 (s, 1H), 10.89 (s, 1H), 8.52 (t, J=6.0 Hz, 1H), 7.61 (d, J=8.1 Hz, 1H), 7.58 (d, J=7.9 Hz, 1H), 7.36 (d, J=8.1 Hz, 1H), 7.22 (t, J=6.0 Hz, 2H), 7.17 (d, J=2.2 Hz, 1H), 7.10-7.03 (m, 1H), 6.97 (dd, J=7.8, 7.1 Hz, 1H), 5.10 (dd, J=13.3, 5.1 Hz, 1H), 4.46-4.19 (m, 4H), 3.65 (t, J=6.6 Hz, 1H), 3.12 (dd, J=14.2, 6.0 Hz, 1H), 2.96-2.87 (m, 2H), 2.60 (d, J=2.0 Hz, 1H), 2.45-2.37 (m, 1H), 2.05-1.96 (m, 1H).

### Example 28: Synthesis of N-((2-(2,6-dioxopiperidin-3-yl)-1-oxoisoindolin-5-yl)methyl)-3-(1H-indol-3-yl)propenamide (36)

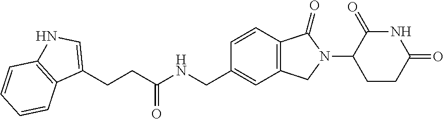 [SMILES: O=C(CCc1c[nH]c2ccccc12)NCc1ccc2c(c1)CN(C1CCC(=O)NC1=O)C2=O]

N-((2-(2,6-dioxopiperidin-3-yl)-1-oxoisoindolin-5-yl)methyl)-3-(1H-indol-3-yl)propenamide was synthesized using the general procedure shown in Reaction Scheme 1 and Example method 1, above (70% yield), and 3-(5-(aminomethyl)-1-oxoisoindolin-2-yl)piperidine-2,6-dione hydrochloride (40 mg, 0.129 mmol) and 3-Indolepropionic acid (26.9 mg, 0.142 mmol) as starting materials.

LCMS: (ESI+) m/z 445.15 [M+H]⁺

1H NMR (500 MHz, DMSO) δ 11.00 (s, 1H), 10.80 (s, 1H), 8.43 (t, J=6.0 Hz, 1H), 7.64 (d, J=7.6 Hz, 1H), 7.56 (d, J=7.9 Hz, 1H), 7.36 (dt, J=8.1, 0.9 Hz, 1H), 7.28 (d, J=9.9 Hz, 2H), 7.13 (d, J=2.2 Hz, 1H), 7.08 (ddd, J=8.1, 7.0, 1.0 Hz, 1H), 6.98 (ddd, J=7.9, 7.0, 1.0 Hz, 1H), 5.12 (dd, J=13.3, 5.1 Hz, 1H), 4.42-4.32 (m, 3H), 4.24 (d, J=17.3 Hz, 1H), 3.00 (t, J=7.4 Hz, 2H), 2.94 (ddd, J=17.4, 13.9, 5.6 Hz, 2H), 2.64 (ddd, J=17.0, 3.6, 1.9 Hz, 1H), 2.57 (t, J=7.4 Hz, 1H), 2.41 (ddd, J=26.4, 13.3, 4.2 Hz, 1H).

### Example 29: Synthesis of 2-amino-N-((2-(2,6-dioxopiperidin-3-yl)-1-oxoisoindolin-5-yl)methyl)-5-guanidinopentanamide (38)

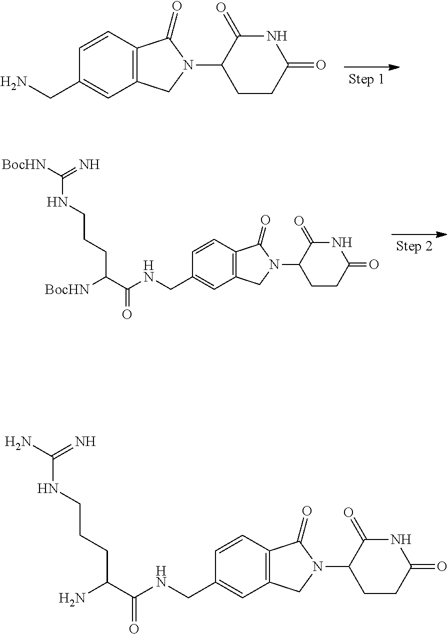 [SMILES: NCc1ccc2c(c1)CN(C1CCC(=O)NC1=O)C2=O ; CC(C)(C)OC(=O)NC(=N)NCCCC(NC(=O)OC(C)(C)C)C(=O)NCc1ccc2c(c1)CN(C1CCC(=O)NC1=O)C2=O ; N=C(N)NCCCC(N)C(=O)NCc1ccc2c(c1)CN(C1CCC(=O)NC1=O)C2=O]

Step 1. tert-butyl N-(4-{[(Z)-{[(tert-butoxy)carbonyl]amino}({[(tert-butoxy)carbonyl]imino})methyl] amino}-1-({[2-(2,6-dioxopiperidin-3-yl)-1-oxo-2,3-dihydro-1H-isoindol-5-yl]methyl}carbamoyl)butyl)carbamate was synthesized using the general procedure shown in Reaction Scheme 1 and Example method 1, above (93% yield), and 3-(5-(aminomethyl)-1-oxoisoindolin-2-yl)piperidine-2,6-dione hydrochloride (40 mg, 0.129 mmol) and (E)-N²,N^{ω},N^{ω}′-tris(tert-butoxycarbonyl)arginine (1.200 eq) as starting materials.

Step 2. 2-amino-N-((2-(2,6-dioxopiperidin-3-yl)-1-oxoisoindolin-5-yl)methyl)-5-guanidinopentanamide was synthesized using the general procedure shown in Reaction Scheme 6 and Example method 6, procedure A, above (50% yield), and tert-butyl N-(4-{[(Z)-{[(tert-butoxy)carbonyl]amino}({[(tert-butoxy)carbonyl]imino})methyl] amino}-1-({[2-(2,6-dioxopiperidin-3-yl)-1-oxo-2,3-dihydro-1H-isoindol-5-yl]methyl}carbamoyl)butyl)carbamate (87.6 mg, 0.120 mmol) as starting material.

LCMS: (ESI+) m/z 430.4 [M+H]⁺

¹H NMR (500 MHz, DMSO) δ 10.98 (s, 1H), 9.02 (q, J=5.6 Hz, 1H), 7.82 (s, 1H), 7.71 (d, J=7.8 Hz, 1H), 7.51 (s, 1H), 7.41 (t, J=14.8 Hz, 1H), 5.11 (dd, J=13.3, 5.1 Hz, 1H), 4.56-4.23 (m, 4H), 3.75 (td, J=6.4, 2.5 Hz, 1H), 3.12 (t, J=6.9 Hz, 2H), 2.92 (ddd, J=17.4, 13.7, 5.4 Hz, 1H), 2.65-2.57 (m, 1H), 2.40 (ddd, J=26.3, 13.2, 4.3 Hz, 1H), 2.01 (ddd, J=10.3, 5.2, 3.1 Hz, 1H), 1.80-1.65 (m, 2H), 1.55-1.46 (m, 2H)

### Example 30: Synthesis of (2S,3R)-2-amino-N-((2-(2,6-dioxopiperidin-3-yl)-1-oxoisoindolin-5-yl)methyl)-3-hydroxybutanamide (39)

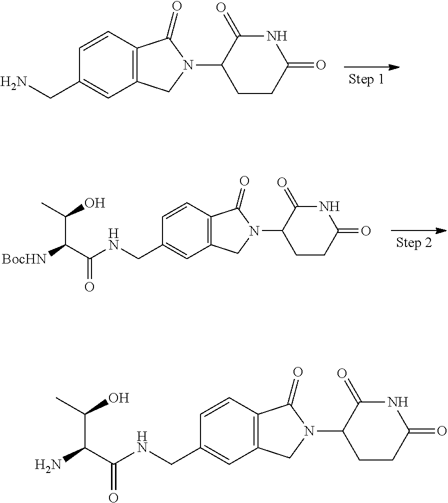 [SMILES: NCc1ccc2c(c1)CN(C1CCC(=O)NC1=O)C2=O ; C[C@@H](O)[C@H](NC(=O)OC(C)(C)C)C(=O)NCc1ccc2c(c1)CN(C1CCC(=O)NC1=O)C2=O ; C[C@@H](O)[C@H](N)C(=O)NCc1ccc2c(c1)CN(C1CCC(=O)NC1=O)C2=O]

Step 1. tert-butyl ((2S,3R)-1-(((2-(2,6-dioxopiperidin-3-yl)-1-oxoisoindolin-5-yl)methyl)amino)-3-hydroxy-1-oxobutan-2-yl)carbamate was synthesized using the general procedure shown in Reaction Scheme 1 and Example method 1, above (69% yield), and 3-(5-(aminomethyl)-1-oxoisoindolin-2-yl)piperidine-2,6-dione hydrochloride (84.8 mg, 0.274 mmol) and (tert-butoxycarbonyl)-L-threonine (1.000 eq) as starting materials.

LCMS: (ESI+) m/z 375.0 [M+H—BOC]⁺; (ESI−) m/z 473.1 [M−H]⁻

¹H NMR (500 MHz, DMSO) δ 10.63 (s, 1H), 8.11 (t, J=5.9 Hz, 1H), 7.64 (dd, J=7.8, 0.7 Hz, 1H), 7.50 (dt, J=1.7, 0.9 Hz, 1H), 7.43 (d, 1H), 6.02 (s, 1H), 5.05 (dd, J=13.0, 5.2 Hz, 1H), 4.56 (dd, J=5.7, 1.1 Hz, 1H), 4.50-4.31 (m, 4H), 4.05-3.88 (m, 2H), 2.88 (ddd, J=17.4, 13.4, 5.5 Hz, 1H), 2.64 (ddd, J=17.4, 4.6, 2.6 Hz, 1H), 2.41 (qd, J=13.0, 4.6 Hz, 1H), 2.08-2.02 (m, 1H), 1.41 (s, 9H), 1.08 (dd, J=10.8, 6.2 Hz, 3H)

Step 2. (2S,3R)-2-amino-N-((2-(2,6-dioxopiperidin-3-yl)-1-oxoisoindolin-5-yl)methyl)-3-hydroxybutanamide was synthesized using the general procedure shown in Reaction Scheme 6 and Example method 6, procedure B, above (100% yield), and tert-butyl ((2S,3R)-1-(((2-(2,6-dioxopiperidin-3-yl)-1-oxoisoindolin-5-yl)methyl)amino)-3-hydroxy-1-oxobutan-2-yl)carbamate (70.5 mg, 0.149 mmol) as starting material.

LCMS: (ESI+) m/z 375.2 [M+H]⁺; (ESI−) m/z 373.2 [M−H]⁻

¹H NMR (500 MHz, DMSO) δ 10.69-10.60 (m, 1H), 8.81 (d, J=6.9 Hz, 1H), 8.04 (s, 3H), 7.69 (d, J=7.8 Hz, 1H), 7.55 (s, 1H), 7.49-7.46 (m, 1H), 5.06 (dd, J=13.0, 5.2 Hz, 1H), 4.49 (d, J=5.8 Hz, 2H), 4.47-4.34 (m, 2H), 4.02 (p, J=6.3 Hz, 1H), 3.66 (d, J=5.9 Hz, 1H), 2.89 (ddd, J=17.3, 13.4, 5.5 Hz, 1H), 2.68-2.61 (m, 1H), 2.42 (qd, J=13.1, 4.7 Hz, 1H), 2.06 (dtd, J=13.0, 5.4, 2.6 Hz, 1H), 1.19 (dd, J=6.4, 0.7 Hz, 3H)

### Example 31: Synthesis of benzyl ((2S,3R)-1-(((2-(2,6-dioxopiperidin-3-yl)-1-oxoisoindolin-5-yl)methyl)amino)-3-hydroxy-1-oxobutan-2-yl)carbamate (40)

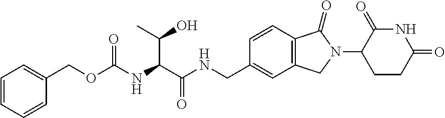 [SMILES: C[C@@H](O)[C@H](NC(=O)OCc1ccccc1)C(=O)NCc1ccc2c(c1)CN(C1CCC(=O)NC1=O)C2=O]

Benzyl (2S,3R)1-(((2-(2,6-dioxopiperidin-3-yl)-1-oxoisoindolin-5-yl)methyl)amino)-3-hydroxy-1-oxobutan-2-yl)carbamate was synthesized using the general procedure shown in Reaction Scheme 1 and Example method 1, above (64% yield), and 3-(5-(aminomethyl)-1-oxoisoindolin-2-yl)piperidine-2,6-dione hydrochloride (30 mg, 0.097 mmol) and (2S,3R)-2-(((benzyloxy)carbonyl)amino)-3-hydroxybutanoic acid (1.200 eq) as starting materials.

LCMS: (ESI+) m/z 508.4 [M+H]⁺; (ESI−) m/z 506.8

¹H NMR (500 MHz, DMSO) δ 10.97 (s, 1H), 8.47 (t, J=5.8 Hz, 1H), 7.65 (d, J=7.8 Hz, 1H), 7.48 (s, 1H), 7.46-7.23 (m, 6H), 6.95 (d, J=8.4 Hz, 1H), 5.16-4.98 (m, 3H), 4.81 (d, J=5.0 Hz, 1H), 4.47-4.21 (m, 4H), 4.06-3.83 (m, 2H), 2.98-2.83 (m, 1H), 2.58 (s, 1H), 2.38 (dd, J=13.9, 5.0 Hz, 1H), 2.05-1.94 (m, 1H), 1.06 (d, J=6.1 Hz, 3H).

### Example 32: Synthesis of benzyl (1-(((2-(2,6-dioxopiperidin-3-yl)-1-oxoisoindolin-5-yl)methyl)amino)-3-(4-hydroxyphenyl)-1-oxopropan-2-yl)carbamate (41)

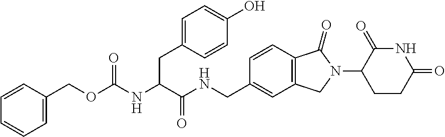 [SMILES: O=C1CCC(N2Cc3cc(CNC(=O)C(Cc4ccc(O)cc4)NC(=O)OCc4ccccc4)ccc3C2=O)C(=O)N1]

This compound was synthesized using the general procedure shown in Reaction Scheme 1 and Example method 1, above (52.7% yield), and 3-(5-(aminomethyl)-1-oxoisoindolin-2-yl)piperidine-2,6-dione hydrochloride (30 mg, 0.097 mmol) and ((benzyloxy)carbonyl)tyrosine (1.200 eq) as starting materials.

LCMS: (ESI+) m/z 570.3 [M+H]⁺; (ESI−) m/z 569.1

¹H NMR (500 MHz, DMSO) δ 10.97 (s, 1H), 9.21 (s, 1H), 8.56 (t, J=6.2 Hz, 1H), 7.63 (d, J=7.8 Hz, 1H), 7.50 (d, J=8.4 Hz, 1H), 7.38-7.22 (m, 6H), 7.05 (d, J=7.8 Hz, 2H), 6.65 (d, J=7.5 Hz, 2H), 5.10 (dd, J=13.3, 4.7 Hz, 1H), 5.03-4.93 (m, 2H), 4.49-4.18 (m, 5H), 2.91 (ddd, J=19.3, 12.8, 5.3 Hz, 2H), 2.77-2.66 (m, 1H), 2.58 (s, 1H), 2.44-2.38 (m, 1H), 2.01 (s, 1H).

### Example 33: Synthesis of N-((2-(2,6-dioxopiperidin-3-yl)-1-oxoisoindolin-5-yl)methyl)-3-(1H-indol-3-yl)propanamide (42)

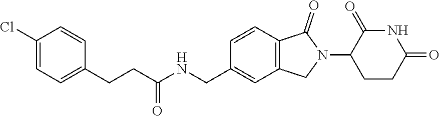 [SMILES: O=C(CCc1ccc(Cl)cc1)NCc1ccc2c(c1)CN(C1CCC(=O)NC1=O)C2=O]

This compound was synthesized using the general procedure shown in Reaction Scheme 1 and Example method 1, above (yield), and 3-(5-(aminomethyl)-1-oxoisoindolin-2-yl)piperidine-2,6-dione hydrochloride (40 mg, 0.129 mmol) and 3-(4-chlorophenyl)propionic acid (26.2 mg, 0.142 mmol) as starting materials.

LCMS: (ESI+) m/z 440.1 [M+H]⁺

1H NMR (500 MHz, DMSO) δ 10.99 (s, 1H), 8.43 (t, J=6.0 Hz, 1H), 7.65 (d, J=7.9 Hz, 1H), 7.38-7.31 (m, 2H), 7.32-7.23 (m, 4H), 5.12 (dd, J=13.3, 5.1 Hz, 1H), 4.41 (d, J=17.2 Hz, 1H), 4.37 (d, J=5.9 Hz, 2H), 4.28 (d, J=17.3 Hz, 1H), 2.94 (ddd, J=17.4, 13.7, 5.4 Hz, 1H), 2.87 (t, J=7.4 Hz, 2H), 2.68-2.59 (m, 1H), 2.55-2.45 (m, 2H), 2.41 (qd, J=13.3, 4.5 Hz, 1H), 2.03 (ddq, J=10.4, 5.2, 2.7 Hz, 1H).

### Example 34: Synthesis of (2S)—N-((2-(2,6-dioxopiperidin-3-yl)-1-oxoisoindolin-5-yl)methyl)pyrrolidine-2-carboxamide hydrochloride (43)

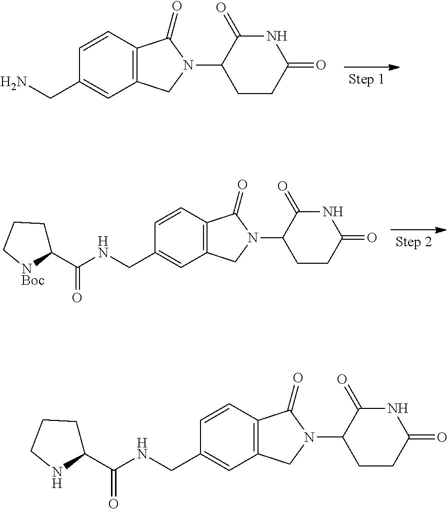

Step 1. This step was performed using the general procedure shown in Reaction Scheme 1 and Example method 1, above (56.3% yield), and 3-(5-(aminomethyl)-1-oxoisoindolin-2-yl)piperidine-2,6-dione hydrochloride (86.3 mg, 0.279 mmol) and (tert-butoxycarbonyl)-L-proline (50 mg, 0.232 mmol) as starting materials.

LCMS: (ESI+) m/z 371.0 [M+H—BOC]⁺; (ESI−) m/z 469.1 [M−H]⁻

¹H NMR (500 MHz, DMSO, T=353K) δ 10.62 (s, 1H), 8.13 (t, 1H), 7.65 (d, J=7.8 Hz, 1H), 7.49 (s, 1H), 7.42 (dd, J=7.8, 1.5 Hz, 1H), 5.05 (dd, J=13.0, 5.2 Hz, 1H), 4.48-4.32 (m, 4H), 4.18-4.12 (m, 1H), 3.42 (ddd, J=10.3, 7.6, 5.3 Hz, 1H), 3.35 (dt, J=10.1, 6.9 Hz, 1H), 2.88 (ddd, J=17.4, 13.4, 5.5 Hz, 1H), 2.64 (ddd, J=17.4, 4.6, 2.6 Hz, 1H), 2.41 (qd, J=13.1, 4.7 Hz, 1H), 2.13 (tt, J=7.9, 3.6 Hz, 1H), 2.08-2.02 (m, 1H), 1.90-1.75 (m, 3H), 1.36 (s, 9H)

Step 2. This step was done using the general procedure shown in Reaction Scheme 6 and Example method 6, procedure B, above (99.6% yield), and tert-butyl (2S)-2-(((2-(2,6-dioxopiperidin-3-yl)-1-oxoisoindolin-5-yl)methyl)carbamoyl)pyrrolidine-1-carboxylate (56.9 mg, 0.121 mmol) as starting material.

LCMS: (ESI+) m/z 371.1 [M+H]⁺

¹H NMR (500 MHz, DMSO) δ 10.98 (s, 1H), 9.61 (d, J=5.1 Hz, 1H), 9.18 (dt, J=6.5, 3.2 Hz, 1H), 8.58 (d, J=5.5 Hz, 1H), 7.71 (d, J=7.8 Hz, 1H), 7.51 (s, 1H), 7.43 (d, J=7.9 Hz, 1H), 5.11 (dd, J=13.3, 5.1 Hz, 1H), 4.47 (d, J=6.0 Hz, 2H), 4.38 (dd, J=66.2, 17.6 Hz, 2H), 4.22 (h, J=6.2 Hz, 1H), 3.22 (ddq, J=23.6, 11.8, 6.4 Hz, 2H), 2.92 (ddd, J=17.3, 13.7, 5.4 Hz, 1H), 2.65-2.52 (m, 1H), 2.45-2.29 (m, 2H), 2.01 (dtd, J=12.7, 5.3, 2.3 Hz, 1H), 1.95-1.84 (m, 3H)

### Example 35: Synthesis of 4-amino-2-(2,6-dioxopiperidin-3-yl)-5-hydroxyisoindoline-1,3-dione (44)

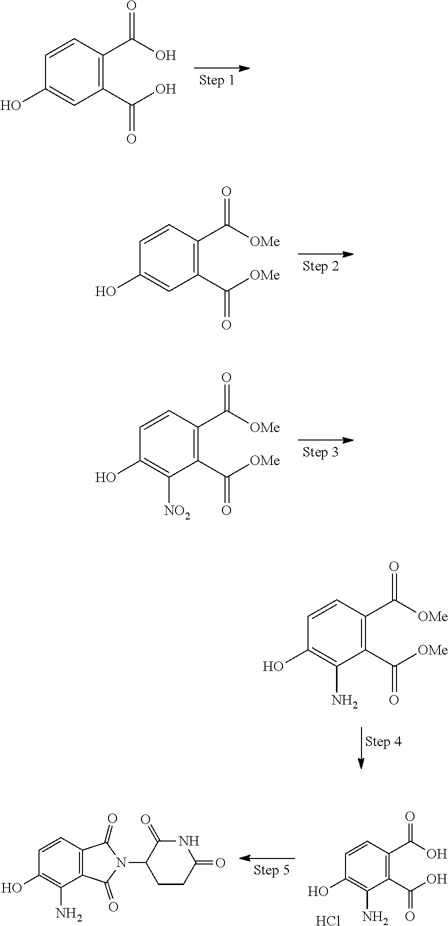

Step 1. To a solution of 4-hydroxyphthalic acid (30 g, 164.7 mmol) in dry MeOH (600 mL) was added concentrated H₂SO₄ (5 mL) and the mixture was refluxed overnight. After cooling to RT, methanol was evaporated, the mixture was diluted with DCM, washed with NaHCO₃ solution and dried over Na₂SO₄. Concentration under reduced pressure gave dimethyl 4-hydroxyphthalate in quantitative yield.

Step 2. To a solution of dimethyl 4-hydroxyphthalate (30 g, 142.7 mmol) in concentrated H₂SO₄ (300 mL), cooled to −10° C., was added dropwise 65% HNO₃ (16.5 mL) and the mixture was stirred at 0° C. for 30 minutes. The reaction mixture was poured onto ice, the product was extracted with EtOAc, washed with water, dried over Na₂SO₄ and concentrated under reduced pressure to obtain the mixture of dimethyl 4-hydroxy-3-nitrophthalate and dimethyl 4-hydroxy-5-nitrophthalate that was separated by column chromatography.

Step 3. To a solution of 4-hydroxy-3-nitrophthalate (5 g, 19.6 mmol) in dry MeOH (100 mL) under argon atmosphere was added Pd/C (5% w.t.). Flask was filled/evacuated with hydrogen three times. The solution was stirred at RT under hydrogen atmosphere (1 bar) for 12 h. After consumption of the starting material, the solvent was evaporated to afford 3.97 g of dimethyl 3-amino-4-hydroxyphthalate (90% yield).

Step 4. A mixture of dimethyl 3-amino-4-hydroxyphthalate (3.97 g, 17.6 mmol) and concentrated aq. HCl (100 mL) was refluxed for 6 h and evaporated under reduced pressure to obtain 2.84 g of 3-amino-4-hydroxyphthalic acid hydrochloride.

Step 5. A mixture of 3-amino-4-hydroxyphthalic acid hydrochloride (2.84 g, 12.1 mmol), 3-aminopiperidine-2,6-dione hydrochloride (2 g, 18.2 mmol), acetonitrile (26 mL), acetic acid (7 mL) and triethylamine (8.3 mL) was refluxed overnight. Then the reaction mixture was cooled to RT, poured into water. The precipitated solid was collected and dried to afford 1.73 g of 4-amino-2-(2,6-dioxopiperidin-3-yl)-5-hydroxyisoindoline-1,3-dione (43% yield).

LCMS: (ESI+) m/z 290.2 [M+H]⁺

¹H NMR (500 MHz, DMSO) δ 11.05 (s, 1H), 10.82 (s, 1H), 6.98 (d, J=7.7 Hz, 1H), 6.92 (d, J=7.7 Hz, 1H), 5.95 (s, 2H), 5.01 (dd, J=12.8, 5.4 Hz, 1H), 2.88 (tdd, J=15.7, 4.6, 2.9 Hz, 1H), 2.63-2.52 (m, 2H), 2.07-1.95 (m, 1H).

### Example 36: Synthesis of 3-(5-(aminomethyl)-1-oxoisoindolin-2-yl)pyrrolidine-2,5-dione (50)

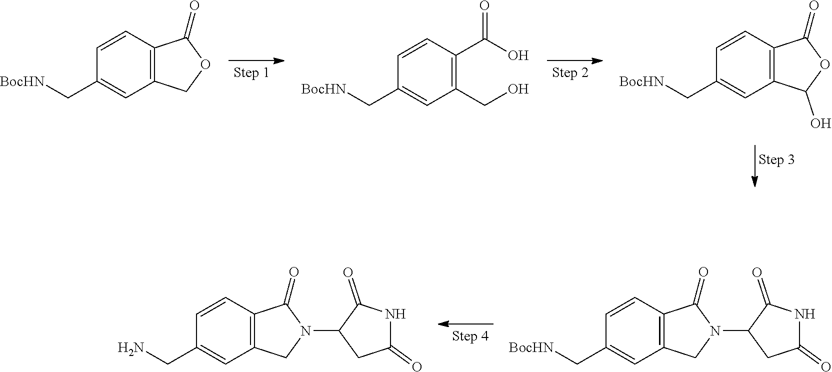 [SMILES: CC(C)(C)OC(=O)NCc1ccc2c(c1)COC2=O ; CC(C)(C)OC(=O)NCc1ccc(C(=O)O)c(CO)c1 ; CC(C)(C)OC(=O)NCc1ccc2c(c1)C(O)OC2=O ; NCc1ccc2c(c1)CN(C1CC(=O)NC1=O)C2=O ; CC(C)(C)OC(=O)NCc1ccc2c(c1)CN(C1CC(=O)NC1=O)C2=O]

Step 1. 4-(((tert-butoxycarbonyl)amino)methyl)-2-(hydroxymethyl)benzoic acid was synthesized using the general procedure shown in Reaction Scheme 2 and Example method 2, above (94% yield), and tert-butyl ((1-oxo-1,3-dihydroisobenzofuran-5-yl)methyl)carbamate (500 mg, 1.9 mmol) as a starting material.

Step 2. tert-butyl ((3-hydroxy-1-oxo-1,3-dihydroisobenzofuran-5-yl)methyl)carbamate was synthesized using the general procedure shown in Reaction Scheme 3 and Example method 3, above (81% yield), and 4-(((tert-butoxycarbonyl)amino)methyl)-2-(hydroxymethyl)benzoic acid (430 mg, 1.53 mmol) as a starting material.

Step 3. tert-butyl ((2-(2,5-dioxopyrrolidin-3-yl)-1-oxoisoindolin-5-yl)methyl)carbamate was synthesized using the general procedure shown in Reaction Scheme 4 and Example method 4, above, and tert-butyl ((3-hydroxy-1-oxo-1,3-dihydroisobenzofuran-5-yl)methyl)carbamate (50 mg, 0.18 mmol) and 3-aminopyrrolidine-2,5-dione hydrochloride (1 eq) as the starting materials.

LCMS: (ESI+) m/z 360.1 [M+H]⁺; (ESI−) m/z 358.1 [M−H]⁻

¹H NMR (500 MHz, DMSO) δ 11.49 (s, 1H), 7.65 (d, J=7.8 Hz, 1H), 7.49 (t, J=6.2 Hz, 1H), 7.44 (s, 1H), 7.40-7.33 (m, 1H), 5.22 (dd, J=9.3, 6.2 Hz, 1H), 4.47 (dd, J=130.4, 17.2 Hz, 2H), 4.23 (d, J=6.2 Hz, 2H), 3.02-2.85 (m, 2H), 1.39 (s, 9H).

Step 4. 3-(5-(aminomethyl)-1-oxoisoindolin-2-yl)pyrrolidine-2,5-dione was synthesized using the general procedure shown in Reaction Scheme 6 and Example method 6, Procedure A, above (12% yield, two steps), and tert-butyl ((2-(2,5-dioxopyrrolidin-3-yl)-1-oxoisoindolin-5-yl)methyl)carbamate (64.3 mg, 0.18 mmol) as a starting material.

LCMS: (ESI−) m/z 258.0 [M−H]⁻

¹H NMR (500 MHz, DMSO) δ 8.94 (s, 1H), 7.75 (d, J=7.8 Hz, 1H), 7.65 (s, 1H), 7.57 (dd, J=7.8, 1.5 Hz, 1H), 5.24 (dd, J=9.2, 6.3 Hz, 1H), 4.51 (dd, J=133.2, 17.4 Hz, 2H), 4.10 (s, 2H), 3.04-2.87 (m, 2H).

### Example 37: 3-(5-(aminomethyl)-1-oxoisoindolin-2-yl)piperidine-2,6-dione-5,5-d₂ (51)

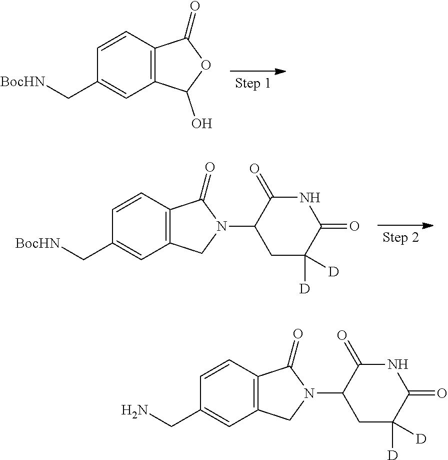

Step 1. Tert-butyl ((2-(2,6-dioxopiperidin-3-yl-5,5-d₂)-1-oxoisoindolin-5-yl)methyl)carbamate was synthesized using the general procedure shown in Reaction Scheme 4 and Example method 4, above (39% yield), and tert-butyl ((3-hydroxy-1-oxo-1,3-dihydroisobenzofuran-5-yl)methyl)carbamate (50 mg, 0.18 mmol) and 3-aminopyrrolidine-2,5-dione-3,5,5-d₃ (1 eq) as the starting materials.

LCMS: (ESI+) m/z 376.3 [M+H]⁺

Step 2. 3-(5-(aminomethyl)-1-oxoisoindolin-2-yl)piperidine-2,6-dione-5,5-d₂ was synthesized using the general procedure shown in Reaction Scheme 6 and Example method 6, Procedure A, above (15% yield), and tert-butyl ((2-(2,6-dioxopiperidin-3-yl-5,5-d2)-1-oxoisoindolin-5-yl)methyl)carbamate (27 mg, 0.073 mmol) as a starting material.

LCMS: (ESI+/ESI-) 276.0 [M+H]⁺

1H NMR (500 MHz, DMSO) δ 10.99 (s, 1H), 8.18 (d, J=45.7 Hz, 3H), 7.80 (d, J=7.8 Hz, 1H), 7.68 (s, 1H), 7.60 (dd, J=7.8, 1.5 Hz, 1H), 5.13 (dd, J=13.4, 5.2 Hz, 1H), 4.52-4.32 (m, 2H), 4.18 (q, J=5.8 Hz, 2H), 2.01 (dd, J=12.6, 5.2 Hz, 1H), 1.29-1.21 (m, 1H).

### Example 38: Synthesis of 3-[5-(aminomethyl)-3-methyl-1-oxo-2,3-dihydro-1H-isoindol-2-yl]piperidine-2,6-dione (52)

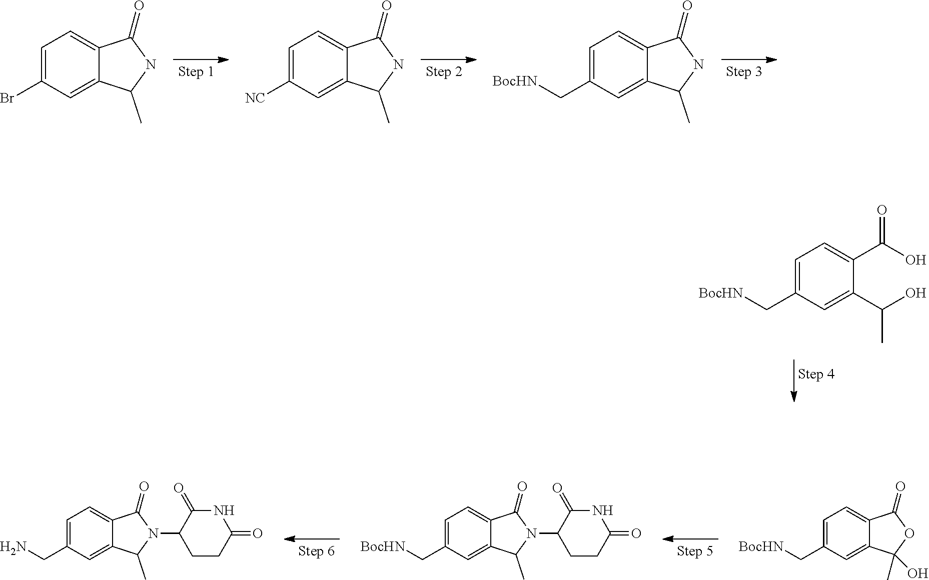

Step 1. To a solution of 5-bromo-6-methylisobenzofuran-1 (3H)-one (500 mg, 2.21 mmol) in DMF (5 mL) was added Zn(CN)₂ (648.7 mg, 5.52 mmol) followed by Pd(PPh₃)₄ (255 mg, 0.221 mmol) and the reaction mixture was heated at 100° C. for 16 h under inert atmosphere. The reaction was quenched with ice water and the product was extracted into EtOAc. The organic layer was dried over Na₂SO₄, concentrated and purified by flash column chromatography to give 314 mg of 3-methyl-1-oxo-1,3-dihydroisobenzofuran-5-carbonitrile (82% yield).

Step 2. To a solution of 3-methyl-1-oxo-1,3-dihydroisobenzofuran-5-carbonitrile (400 mg, 2.30 mmol) in ethanol (5 mL) was added Boc₂O (1.056 mL, 4.598 mmol) followed by Raney Ni (80 mg) and the reaction mixture was stirred under hydrogen atmosphere (1 bar) for 16 h. The reaction mixture was filtered, concentrated under reduced pressure. The crude was purified by flash column chromatography to give 320 mg of tert-butyl ((3-methyl-1-oxo-1,3-dihydroisobenzofuran-5-yl)methyl)carbamate (50% yield).

Step 3. 4-(((tert-butoxycarbonyl)amino)methyl)-2-(1-hydroxyethyl)benzoic acid was synthesized using the general procedure shown in Reaction Scheme 2 and Example method 2, above (99.8% yield), and tert-butyl N-[(3-methyl-1-oxo-1,3-dihydro-2-benzofuran-5-yl)methyl]carbamate (32.0 mg, 0.115 mmol) as a starting material.

Step 4. tert-butyl N-[(3-hydroxy-3-methyl-1-oxo-1,3-dihydro-2-benzofuran-5-yl)methyl]carbamate was synthesized using the general procedure shown in Reaction Scheme 3 and Example method 3, above (80.0% yield), and 4-({[(tert-butoxy)carbonyl]amino}methyl)-2-(1-hydroxyethyl)benzoic acid (33.5 mg, 0.114 mmol), as a starting material.

Step 5. tert-butyl N-{[2-(2,6-dioxopiperidin-3-yl)-3-methyl-1-oxo-2,3-dihydro-1H-isoindol-5-yl]methyl}carbamate was synthesized using the general procedure shown in Reaction Scheme 4 and Example method 4, above (4.0% yield), and tert-butyl N-[(3-hydroxy-3-methyl-1-oxo-1,3-dihydro-2-benzofuran-5-yl)methyl]carbamate (33.3 mg, 0.091 mmol) as a starting material.

Step 6. 3-[5-(aminomethyl)-3-methyl-1-oxo-2,3-dihydro-1H-isoindol-2-yl]piperidine-2,6-dione was synthesized using the general procedure shown in Reaction Scheme 6 and Example method 6, procedure B, above (100% yield), and tert-butyl N-{[2-(2,6-dioxopiperidin-3-yl)-3-methyl-1-oxo-2,3-dihydro-1H-isoindol-5-yl]methyl}carbamate (1.4 mg, 0.004 mmol) as starting material.

LCMS: (ESI+) m/z 288.1 [M+H]⁺

¹H NMR (500 MHz, DMSO) δ 10.59 (s, 1H), 8.38 (s, 3H), 7.76-7.69 (m, 2H), 7.61 (dd, J=7.7, 5.3 Hz, 1H), 4.73 (td, J=12.4, 5.2 Hz, 1H), 4.17 (s, 2H), 2.84-2.73 (m, 1H), 2.69-2.56 (m, 2H), 2.11-2.02 (m, 1H), 1.48 (dd, J=13.0, 6.7 Hz, 3H).

### Example 39: Synthesis of 2-(2,6-dioxopiperidin-3-yl)-6-fluoro-1-oxo-2,3-dihydro-1H-isoindole-5-carboxylic acid (54)

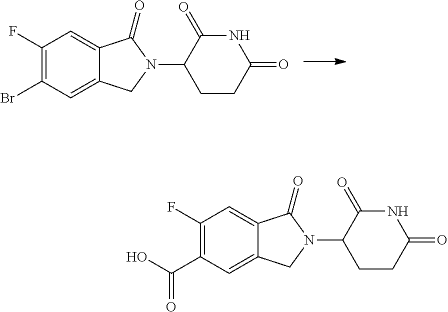 [SMILES: O=C1CCC(N2Cc3cc(Br)c(F)cc3C2=O)C(=O)N1 ; O=C1CCC(N2Cc3cc(C(=O)O)c(F)cc3C2=O)C(=O)N1]

In a vial were placed 3-(5-bromo-6-fluoro-1-oxo-2,3-dihydro-1H-isoindol-2-yl)piperidine-2,6-dione (50.0 mg, 0.147 mmol), MO(CO)₆ (0.035 mL, 0.256 mmol), DMAP (35.8 mg, 0.293 mmol), Pd₂(dba)₃ (13.4 mg, 0.015 mmol), tri-tert-butylphosphane trifluoroborate (8.5 mg, 0.029 mmol), 1,4-dioxane (2.0 mL), H₂O (0.200 mL) and DIPEA (51 μL, 0.293 mmol). Reaction was carried out at 150° C. for 30 minutes in the microwave reactor. The crude product was purified by preparative HPLC to give 13.0 mg of 2-(2,6-dioxopiperidin-3-yl)-6-fluoro-1-oxo-2,3-dihydro-1H-isoindole-5-carboxylic acid (28% yield) as a white solid.

LCMS: (ESI+) m/z 307.0 [M+H]⁺; (ESI−) m/z 304.9 [M−H]⁻

¹H NMR (500 MHz, DMSO) δ 13.58 (s, 1H), 11.01 (s, 1H), 8.08 (d, J=6.1 Hz, 1H), 7.62 (d, J=9.3 Hz, 1H), 5.14 (dd, J=13.3, 5.1 Hz, 1H), 4.50 (d, J=17.3 Hz, 1H), 4.38 (d, J=17.3 Hz, 1H), 2.91 (ddd, J=17.3, 13.7, 5.5 Hz, 1H), 2.60 (ddd, J=17.1, 5.0, 2.5 Hz, 1H), 2.41 (td, J=13.1, 4.5 Hz, 1H), 2.03 (dtd, J=12.8, 5.4, 2.3 Hz, 1H)

### Example 40: Synthesis of (S)-3-(5-(aminomethyl)-1-oxoisoindolin-2-yl)-3-methylpiperidine-2,6-dione (48)

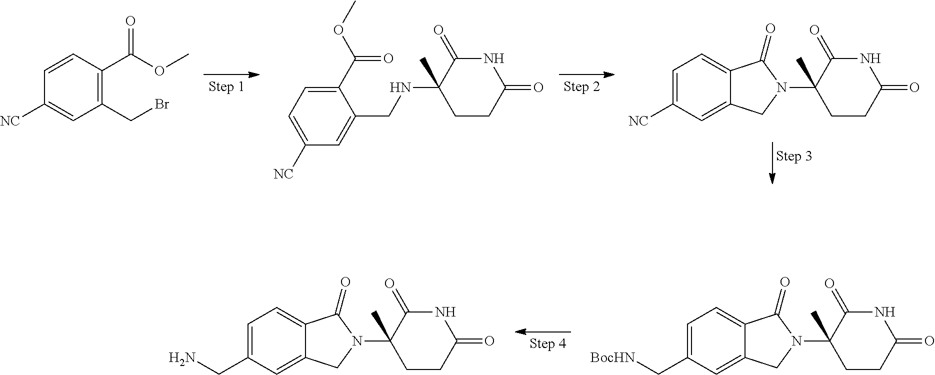 [SMILES: COC(=O)c1ccc(C#N)cc1CBr ; COC(=O)c1ccc(C#N)cc1CN[C@@]1(C)CCC(=O)NC1=O ; C[C@]1(N2Cc3cc(C#N)ccc3C2=O)CCC(=O)NC1=O ; C[C@]1(N2Cc3cc(CN)ccc3C2=O)CCC(=O)NC1=O ; CC(C)(C)OC(=O)NCc1ccc2c(c1)CN([C@@]1(C)CCC(=O)NC1=O)C2=O]

Step 1. To a solution of methyl 2-bromomethyl-4-cyanobenzoate (114.0 mg, 0.448 mmol) and (S)-3-amino-3-methylpiperidine-2,6-dione hydrobromide (100.0 mg, 0.448 mmol) in ACN (6 mL) was added DIPEA (0.390 mL, 2.242 mmol) and the reaction mixture was stirred at RT for 18 h. The volatiles were removed under reduced pressure and the residue was purified by preparative HPLC to give 63.0 mg of methyl (S)-4-cyano-2-(((3-methyl-2,6-dioxopiperidin-3-yl)amino)methyl)benzoate (47% yield).

Step 2. To a suspension of (S)-4-cyano-2-(((3-methyl-2,6-dioxopiperidin-3-yl)amino)methyl)benzoate (67.0 mg, 0.212 mmol) in dry toluene (6 mL) was added bis(trimethylaluminum)-1,4-diazabicyclo[2.2.2]octane adduct (5.4 mg, 0.021 mmol) and the reaction mixture was refluxed for 12 h. The volatiles were removed under reduced pressure and the residue was purified by preparative HPLC to give 45 mg of (S)-2-(3-methyl-2,6-dioxopiperidin-3-yl)-1-oxoisoindoline-5-carbonitrile (74% yield).

Step 3. To a solution of (S)-2-(3-methyl-2,6-dioxopiperidin-3-yl)-1-oxoisoindoline-5-carbonitrile (25.0 mg, 0.088 mmol) and Boc₂O (38.5 mg, 0.176 mmol) in a mixture of DMF (1.5 mL) and THE (2.5 mL) was added Raney Nickel (33 mg) and the reaction mixture was stirred under hydrogen (1 bar) for 24 h. The reaction mixture was filtered, concentrated under reduced pressure and the residue was purified by preparative HPLC to give 18.2 mg of tert-butyl (S)-((2-(3-methyl-2,6-dioxopiperidin-3-yl)-1-oxoisoindolin-5-yl)methyl)carbamate (53% yield).

Step 4. (S)-3-(5-(aminomethyl)-1-oxoisoindolin-2-yl)-3-methylpiperidine-2,6-dione was synthesized using the general procedure shown in Reaction Scheme 6 and Example method 6, Procedure A, above (90% yield), and tert-butyl (S)-((2-(3-methyl-2,6-dioxopiperidin-3-yl)-1-oxoisoindolin-5-yl)methyl)carbamate (18.2 mg, 0.047 mmol) as a starting material.

LCMS: (ESI+) m/z 288.1 [M+H]⁺

¹H NMR (500 MHz, DMSO) δ 10.88 (s, 1H), 7.67 (d, J=7.8 Hz, 1H), 7.64 (s, 1H), 7.55 (d, J=7.8 Hz, 1H), 7.16 (s, 2H), 4.73 (d, J=17.5 Hz, 1H), 4.67 (d, J=17.5 Hz, 1H), 4.06 (s, 2H), 2.84-2.62 (m, 2H), 2.58-2.53 (m, 1H), 1.92 (dq, J=12.9, 5.1, 4.2 Hz, 1H), 1.71 (s, 3H).

### Example 41: Synthesis of (R)-3-(5-(aminomethyl)-1-oxoisoindolin-2-yl)-3-methylpiperidine-2,6-dione (49)

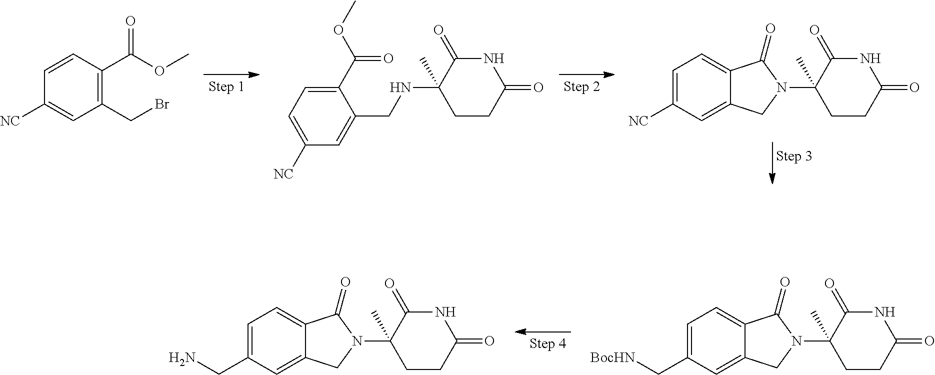 [SMILES: COC(=O)c1ccc(C#N)cc1CBr ; COC(=O)c1ccc(C#N)cc1CN[C@]1(C)CCC(=O)NC1=O ; C[C@@]1(N2Cc3cc(C#N)ccc3C2=O)CCC(=O)NC1=O ; C[C@@]1(N2Cc3cc(CN)ccc3C2=O)CCC(=O)NC1=O ; CC(C)(C)OC(=O)NCc1ccc2c(c1)CN([C@]1(C)CCC(=O)NC1=O)C2=O]

Step 1. To a solution of methyl 2-bromomethyl-4-cyanobenzoate (57.0 mg, 0.224 mmol) and (R)-3-amino-3-methylpiperidine-2,6-dione hydrobromide (50.0 mg, 0.224 mmol) in ACN (3 mL) was added DIPEA (0.195 mL, 1.121 mmol) and the reaction mixture was stirred at RT for 18 h. The volatiles were removed under reduced pressure and the residue was purified by preparative HPLC to give 37.0 mg of methyl (R)-4-cyano-2-(((3-methyl-2,6-dioxopiperidin-3-yl)amino)methyl)benzoate (52% yield).

Step 2. To a suspension of (R)-4-cyano-2-(((3-methyl-2,6-dioxopiperidin-3-yl)amino)methyl)benzoate (37.0 mg, 0.117 mmol) in dry toluene (3 mL) was added bis(trimethylaluminum)-1,4-diazabicyclo[2.2.2]octane adduct (3.0 mg, 0.012 mmol) and the reaction mixture was refluxed for 12 h. The volatiles were removed under reduced pressure and the residue was purified by preparative HPLC to give 17 mg of (R)-2-(3-methyl-2,6-dioxopiperidin-3-yl)-1-oxoisoindoline-5-carbonitrile (51% yield).

Step 3. To a solution of (R)-2-(3-methyl-2,6-dioxopiperidin-3-yl)-1-oxoisoindoline-5-carbonitrile (15.0 mg, 0.053 mmol) and Boc₂O (23.1 mg, 0.106 mmol) in a mixture of DMF (1.0 mL) and THE (1.5 mL) was added Raney Nickel (20 mg) and the reaction mixture was stirred under hydrogen (1 bar) for 24 h. The reaction mixture was filtered, concentrated under reduced pressure and the residue was purified by preparative HPLC to give 11.1 mg of tert-butyl (R)-((2-(3-methyl-2,6-dioxopiperidin-3-yl)-1-oxoisoindolin-5-yl)methyl)carbamate (54% yield).

Step 4. (R)-3-(5-(aminomethyl)-1-oxoisoindolin-2-yl)-3-methylpiperidine-2,6-dione was synthesized using the general procedure shown in Reaction Scheme 6 and Example method 6, Procedure A, above (72% yield), and tert-butyl (R)-((2-(3-methyl-2,6-dioxopiperidin-3-yl)-1-oxoisoindolin-5-yl)methyl)carbamate (11.1 mg, 0.029 mmol) as a starting material.

LCMS: (ESI+) m/z 288.1 [M+H]⁺

¹H NMR (500 MHz, DMSO) δ 10.87 (s, 1H), 7.66 (d, J=7.8 Hz, 1H), 7.64 (s, 1H), 7.54 (d, J=7.8 Hz, 1H), 7.02 (br s, 2H), 4.72 (d, J=17.5 Hz, 1H), 4.66 (d, J=17.5 Hz, 1H), 4.04 (s, 2H), 2.82-2.64 (m, 2H), 2.59-2.51 (m, 1H), 1.92 (dq, J=12.9, 5.2, 4.2 Hz, 1H), 1.71 (s, 3H).

### Examples 42-50: Degradation Assays, Cell Viability Assays, and Cell Survival Assays

TABLE 2
Reference compounds ID and chemical structures.
Reference Compounds

| Compound ID | Structure |
|---|---|
| Thalidomide | 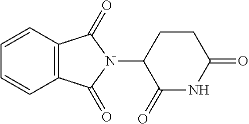 [SMILES: O=C1CCC(N2C(=O)c3ccccc3C2=O)C(=O)N1] |
| Pomalidomide | 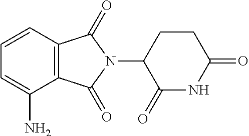 [SMILES: Nc1cccc2c1C(=O)N(C1CCC(=O)NC1=O)C2=O] |
| Lenalidomide | 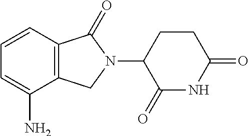 [SMILES: Nc1cccc2c1CN(C1CCC(=O)NC1=O)C2=O] |
| CC-122 | 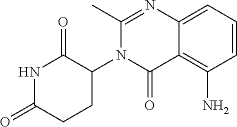 [SMILES: Cc1nc2cccc(N)c2c(=O)n1C1CCC(=O)NC1=O] |
| CC-220 | 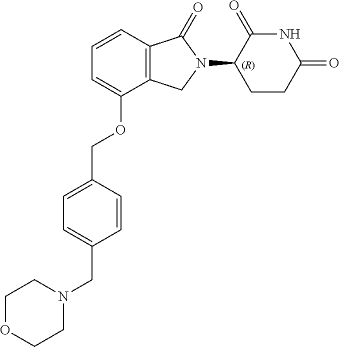 |
| CC-885 | 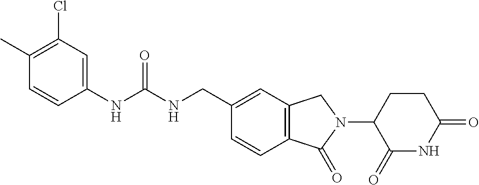 [SMILES: Cc1ccc(NC(=O)NCc2ccc3c(c2)CN(C2CCC(=O)NC2=O)C3=O)cc1Cl] |
| CC-90009 | 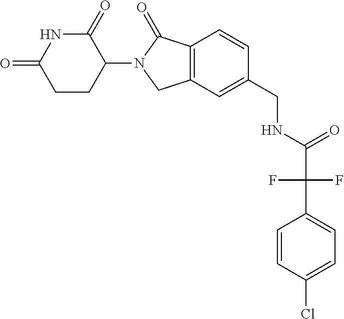 [SMILES: O=C1CCC(N2Cc3cc(CNC(=O)C(F)(F)c4ccc(Cl)cc4)ccc3C2=O)C(=O)N1] |
| 100 | 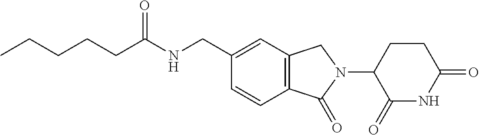 [SMILES: CCCCCC(=O)NCc1ccc2c(c1)CN(C1CCC(=O)NC1=O)C2=O] |

### Example 42: Fluorescence Polarization (FP) Assays

CRBN-DDB1 protein complex was mixed with Cy5-labelled thalidomide and a compound to be tested (the “test compound”). The test solution contained 50 mM Tris pH=7.0, 200 mM NaCl, 0.02% v/v Tween-20, 2 mM DTT, 5 nM Cy5-labelled thalidomide (the tracer), 25 nM CRBN-DDB1 protein, 2% v/v DMSO. The test solution was added to a 384-well assay plate. The plate was spun-down (1 min, 1000 rpm, 22° C.) and then shaken using a VibroTurbulator for 10 min at room temperature (20-25° C.), with the frequency set to level 3. The assay plate with protein and the tracer was incubated for 60 min at room temperature (20-25° C.) prior to read-out with a plate reader. Read-out (fluorescence polarization) was performed by a Pherastar plate reader, using a Cy5 FP Filterset (590 nm/675 nm).

The FP experiment was carried out with various concentrations of the test compounds in order to measure Kᵢ values.

The Kᵢ values of competitive inhibitors were calculated using the equation based on the IC₅₀ values of relationship between compound concentration and measured fluorescence polarization, the K_{d} value of the Cy5-T and CRBN/DDB1 complex, and the concentrations of the protein and the tracer in the displacement assay (as described by Z. Nikolovska-Coleska et al., Analytical Biochemistry 332 (2004) 261-273).

### Fluorescence Polarization (FP) Assay—Results

Compounds are categorized based on their affinity to CRBN defined as Ki. As reported in Table 3 below, the compounds of the present invention interact with CRBN-DDB1 protein within similar affinity range as reported for reference compounds.

CRBN binding Ki [μM] is indicated as follows:

$$A < 0.5 μM$$

$$0.5 μM \leq B \leq 1 μM$$

TABLE 3
Fluorescence Polarization (FP) Assay

| | CRBN binding Ki |
|---|---|
| Compound ID | [μM] |
| 1 | A |
| 12 | B |
| 14 | A |
| 30 | B |
| 29 | B |
| 28 | A |
| 27 | A |
| 24 | A |
| 31 | A |
| 35 | A |
| 11 | A |
| 10 | A |
| 32 | A |
| 41 | A |
| 40 | A |
| 23 | A |
| 22 | A |
| 9 | A |
| 8 | A |
| 33 | A |
| 17 | A |
| 37 | A |
| 34 | A |
| 43 | A |
| 39 | A |
| 16 | A |
| 26 | A |
| 38 | A |
| 25 | A |
| 21 | A |
| 19 | A |
| 20 | A |
| 15 | A |
| 3 | B |
| 2 | A |
| 36 | A |
| 42 | A |
| 6 | A |
| 5 | B |
| 4 | B |
| 7 | B |
| 13 | A |

### Example 43: SALL4 Degradation Assay—Kelly Cell Line

The effect of various compounds of the invention and various reference compounds on SALL4 degradation in the Kelly cell line was investigated, using the degradation assay protocol below.

Kelly cells were maintained in RPMI-1640 medium, supplemented with penicillin/streptomycin and 10% Fetal Bovine Serum (FBS). Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The results for the 24 h treatment with 100 nM compounds are shown in Table 4 below.

TABLE 4
% of SALL4 protein reduction after the treatment of Kelly cells
with compounds of the invention and the reference compound
thalidomide. Average from n ≥ 2 experiments is shown.

| Compound | % of protein reduction |
|---|---|
| 2 | >80% |
| 21 | >80% |
| 23 | >80% |
| 27 | >80% |
| 1 | >80% |
| 44 | >80% |
| Thalidomide | 53% |
| Lenalidomide | 65% |

Thalidomide, Lenalidomide, 1 and 44 were also tested at concentrations of 0.01-1 μM for 24 h. As illustrated in FIG. 1, the compounds of the invention induce potent degradation of SALL4 (>50%) at low concentration (0.01 μM), while lenalidomide and thalidomide have lower activity.

As illustrated in Table 4 and FIG. 1, the compounds of the invention induce degradation of SALL4 protein in the Kelly (neuroblastoma) cell line at lower concentration than the reference compounds. The compounds of the present invention may therefore be useful as anti-cancer drug candidates.

### Example 44: SALL4 Degradation in the Kelly Cell Line—Time Course

The time course of s SALL4 degradation in the Kelly cell line upon treatment with various compounds of the invention and various reference compounds was also analyzed.

Kelly cells were maintained in RPMI-1640 medium, supplemented with penicillin/streptomycin and 10% Fetal Bovine Serum (FBS). Cells were seeded on 6- or 12-well plates, and the compounds to be tested were added at the desired concentration range. Final DMSO concentration was 0.25%. After incubation (37° C., 5% CO₂) for a specified period of time, cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The compounds tested in this assay were Lenalidomide, 1 and 44 at the concentration of 0.1 μM for 3, 6, 12, 24, 48 and 72 h. The results are shown in FIG. 2. As shown in this Figure, the compounds of the present invention degraded SALL4 more rapidly and effectively than lenalidomide, which suggests that the inventive compounds could be administered at lower doses than the reference compounds.

### Example 45: GSPT1 Degradation Assay—Hep3B Cell Line

The effect of various compounds of the invention and various reference compounds on GSPT1 degradation in the Hep3B cell line was investigated, using the degradation assay protocol below.

Hep3B cells were maintained in EMEM medium, supplemented with penicillin/streptomycin and 10% Fetal Bovine Serum (FBS). Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process. Densitometry values were normalized to the loading control and calculated as [%] of the DMSO control.

The results for the 24 h treatment with 1 uM and 10 uM compounds are shown in Table 5 below.

TABLE 5
% of GSPT1 protein reduction after the treatment
of HEP3B cells with compounds of the invention

% of protein reduction

| Compound | 1 uM | 10 uM |
|---|---|---|
| 54 | | A |
| 7 | | B |
| 5 | | A |
| 52 | | A |
| 2 | | A |
| 3 | | A |
| 20 | | B |
| 19 | | B |
| 53 | B | A |
| 27 | A | |
| 28 | A | |
| 1 | A | A |
| 44 | B | |

A represents 80-100% of GSPT1 protein reduction, B represents 50-79% GSPT1 protein reduction

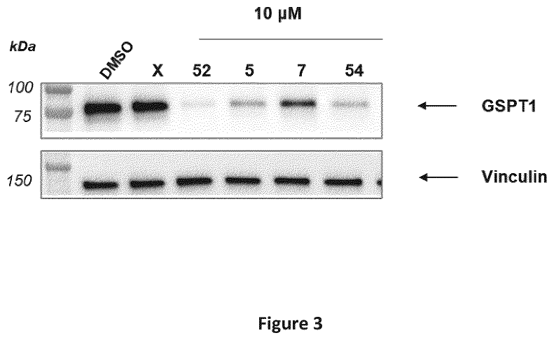

As illustrated in Table 5 and FIG. 3, the compounds of the invention induce degradation of GSPT1 protein in the Hep3B cell line. The compounds of the present invention may therefore be useful as anti-cancer drug candidates.

### Example 46: Ikaros Degradation Assay—H929 Cell Line

The effect of various compounds of the invention and various reference compounds on Ikaros degradation in the H929 cell line was investigated, using the degradation assay protocol above.

H929 cells were maintained in RPMI-1640 medium, supplemented with penicillin/streptomycin and 10% Fetal Bovine Serum (FBS). Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The results for the 24 h treatment with 10 μM or 20 μM compounds are shown in Table 6 below.

TABLE 6
% of IKZF1 protein reduction after the treatment of H929 cells
with compounds of the invention and the reference compounds

% of protein reduction

| Compound | 10 uM | 20 uM |
|---|---|---|
| 54 | C | |
| 7 | C | |
| 4 | C | |
| 5 | C | |
| 52 | C | |
| 2 | C | |
| 3 | C | |
| 25 | | C |
| 26 | | C |
| 16 | C | |
| 39 | | C |
| 43 | | C |
| 34 | | C |
| 37 | | C |
| 17 | C | |
| 33 | | C |
| 8 | | C |
| 9 | | C |
| 23 | | C |
| 32 | | C |
| 10 | C | |
| 11 | | C |
| 31 | | C |
| 24 | | C |
| 27 | C | |
| 28 | C | |
| 29 | | C |
| 30 | | C |
| 1 | C | C |
| 44 | C | |
| Pomalidomide | 86% | 91% |
| Lenalidomide | 66% | 70% |
| CC-885 | >99% | >99% |
| CC-90009 | 33% | — |
| 100 | 52% | — |

C represents 0-24% of IKZF1 protein reduction

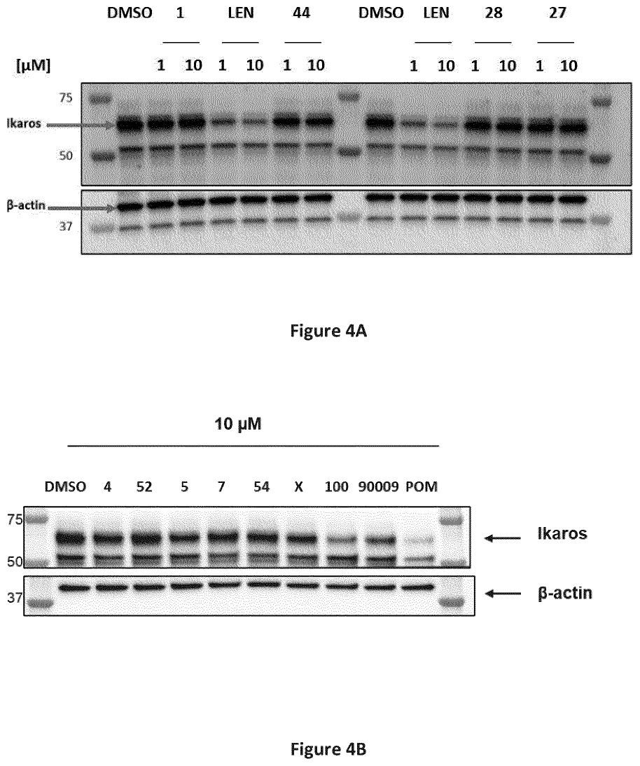

Lenalidomide, 1, 44, 28, and 27 were also tested at the concentrations 1 and 10 μM for 24 h. The results are shown in FIG. 4A.

Compounds 4, 52, 5, 7, and 54 of the present invention were also tested at 10 μM concentration for 24 h together with the reference compounds 100, CC-90009 and Pomalidomide. The results are shown in FIG. 4B.

As illustrated with the examples, the compounds of the present invention were less potent than the reference compounds against Ikaros (IKZF1).

### Example 47: Aiolos Degradation Assay—H929 Cell Line

The effect of various compounds of the invention and various reference compounds on Aiolos degradation in the H929 cell line was investigated, using the degradation assay protocol below.

H929 cells were maintained in RPMI-1640 medium, supplemented with penicillin/streptomycin and 10% Fetal Bovine Serum (FBS). Cells were seeded on 6- or 12-well plates, and the compounds to be tested were added at the desired concentration range. Final DMSO concentration was 0.25%. After 24 h incubation (37° C., 5% CO₂), cells were harvested, washed and cell lysates were prepared using RIPA lysis buffer. The amount of protein was determined via BCA assay, and the appropriate quantity was then loaded on the precast gel for the protein separation. After primary and secondary Ab staining, the membranes were washed and signals developed. The densitometry analysis was implemented to obtain the numeric values used later in the protein level evaluation process.

The results for the 24 h treatment with 20 μM compounds are shown in Table 7 below.

TABLE 7
% of IKZF3 protein reduction after the treatment of H929 cells
with compounds of the invention and the reference compounds

| Compound | % of protein reduction |
|---|---|
| 27 | C |
| 28 | C |
| 29 | C |
| 1 | C |
| Pomalidomide | 87% |
| Lenalidomide | 57% |
| 100 | 52% |

C represents 0-24% of IKZF1 protein reduction

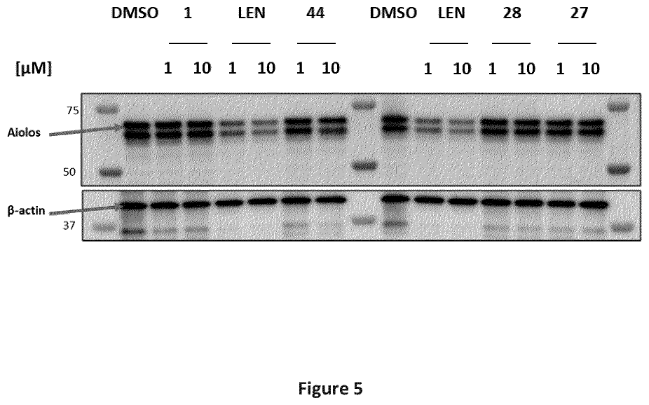

Lenalidomide, 1, 44, 28 and 27 were also tested at concentrations 1 and 10 μM for 24 h. Densitometry values were normalized to the loading control and calculated as [%] of the DMSO control. The results are shown in FIG. 5. As illustrated with the examples, the compounds of the present invention were less potent than the reference compounds against Aiolos (IKZF3).

The compounds of the present invention have a unique degradation profile, as they induce potent degradation of some proteins such as oncogenic SALL4 and GSPT1 proteins (FIGS. 1-3), but are inactive or less potent against Ikaros and Aiolos (FIGS. 4-5).

### Example 48: Cell Viability in Hep3B, Kelly, H929, KG-1 and SNU-398 Cell Lines

The effect of various compounds of the invention and various reference compounds on cell viability in various cell lines was investigated, using the Cell Viability—CTG Assay Protocol below. Hep3B, Kelly, H929, KG-1 and SNU-398 cells were maintained in respective cell medium (see Table 8, below). The cells were seeded on the 96-well or 384-white plates, and the compounds to be tested were added at the desired concentration range. Compounds are diluted in DMSO, and the DMSO concentration is kept constant at 0.25% v/v across the assay plate. After 72 h incubation (37° C., 5% CO₂), CellTiter-Glo® Reagent (Promega/G7570) was added to the wells. Plates are shaken for 4 minutes and incubated for 8 minutes in the dark prior to reading luminescence (LU) by using the CLARIOstar Multimode Plate Reader. The signal is proportional to the amount of ATP, which is directly proportional to the number of cells present in the culture.

Luminescence (RLU) values were normalized to DMSO control. Dose response is assessed by non-linear regression and IC50 calculation with the average of technical duplicate of percentage inhibition. IC50 values are reported as absolute IC50 values, being the concentration of test compound at the intersection of the concentration-response curves with T/C=50%. For calculation of mean IC50 values the geometric mean is used.

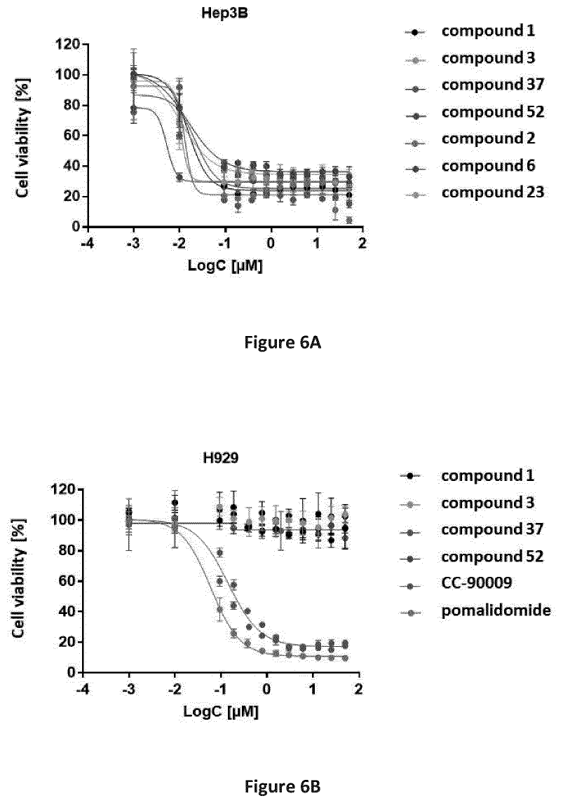

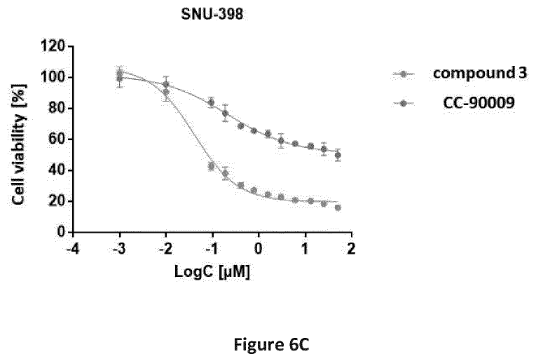

The compounds tested in KG-1, Kelly, and Hep3B cells assay are listed in Table 9. The compounds were tested at the range of concentrations 0.001-50 μM for 72 h. Absolute IC50 values are displayed in Table 9. Dose response plot in Hep3B cells for representative compounds 1, 2, 3, 6, 23, 37, and 52 is shown in FIG. 6A. As shown in this Figure and in Table 9, the compounds of the present invention exhibit potent anticancer activity in KG-1, Kelly, and Hep3B cells derived from leukemia, neuroblastoma and hepatocellular carcinoma, respectively.

The compounds tested in H929 cells assay are listed in Table 10. The compounds were tested at the range of concentrations 0.001-50 μM for 72 h. Luminescence (RLU) values were normalized to DMSO control. Absolute IC50 values are displayed in Table 10. Dose response plot for representative compounds 1, 3, 37, and 52 of the invention and reference compounds CC-90009 and pomalidomide are shown in FIG. 6B. As shown in this Figure, in H929 cell line compounds of the present invention exhibited no to minor activity, while reference compounds potently inhibited growth of the cells.

The compounds tested in SNU-398 cells assay were compound 3 of the invention and a reference clinical stage compound CC-90009 at the range of concentrations 0.001-50 μM for 72 h. Luminescence (RLU) values were normalized to DMSO control. The results are shown in FIG. 6C. As shown in this Figure, in SNU-398 cell line derived from hepatocellular carcinoma the growth of the cells was potently inhibited by the compound of the present invention (IC50=82 nM), while CC-90009 displayed minor activity (IC50>9.9 μM).

TABLE 8
Cell lines and culture media.

| | | Growth | |
|---|---|---|---|
| Cell line | Source (ref#) | property | Culture medium |
| Hep3B | ATCC (HB-8064) | Adherent | 89% Eagle's Minimum Essential Medium (EMEM) |
| | | | 10% Fetal Bovine Serum, qualified, heat inactivated |
| | | | 1% Penicillin/Streptomycin |
| Kelly | DSMZ (ACC355) | Adherent | 89% RPMI 1640 Medium |
| | | | 10% Fetal Bovine Serum, qualified, heat inactivated |
| | | | 1% Penicillin/Streptomycin |
| H929 | ATCC (CRL-9068) | non-adherent | 89% RPMI 1640 (ATCC modification) |
| | | | 10% Fetal Bovine Serum, qualified, heat inactivated |
| | | | 1% Penicillin/Streptomycin |
| | | | 0.05 mM |
| | | | β-Mercaptoethanol |
| KG-1 | DMSZ (ACC14) | non-adherent | 89% RPMI 1640 Medium |
| | | | 10% Fetal Bovine Serum, qualified, heat inactivated |
| | | | 1% Penicillin/Streptomycin |
| SNU-398 | ATCC, CRL-2233 | Adherent | 89% RPMI 1640 Medium (Gibco, 21875-034) |
| | | | 10% Fetal Bovine Serum, qualified, heat inactivated |
| | | | (Gibco, 10500-064) |
| | | | 1% Penicillin/Streptomycin (Biowest, L0022-100) |

TABLE 9
Cell viability following treatment with compounds of the invention

| | KG-1 Geomean | Kelly Geomean | Hep3B Geomean |
|---|---|---|---|
| | absolute IC50 | absolute IC50 | absolute IC50 |
| | by molecule, | by molecule, | by molecule, |
| | 72 H | 72 H | 72 H |
| Compound | [μM] | [nM] | [μM] |
| 1 | A | B | A |
| 28 | B | B | B |
| 27 | B | B | B |
| 24 | A | C | A |
| 31 | C | C | B |
| 23 | | | A |
| 22 | A | B | A |
| 37 | B | B | A |
| 43 | C | | B |
| 39 | C | C | B |
| 26 | | | A |
| 38 | | | B |
| 25 | C | C | B |
| 21 | C | C | A |
| 3 | A | A | A |
| 2 | B | B | A |
| 36 | A | A | A |
| 42 | A | A | A |
| 6 | | | A |
| 52 | | | A |
| 5 | | | B |

A represents IC50 ≤ 100 nM, B represents 1 μM ≥ IC50 > 100 nM, C represents 5 μM ≥ IC50 > 1 μM

TABLE 10
Viability of H929 cells following treatment with compounds
of the invention and the reference compounds

| | Geomean absolute IC50 |
|---|---|
| | by molecule, 72 H |
| Compound | [uM] |
| 1 | Inactive |
| 28 | Inactive |
| 27 | Inactive |
| 24 | Inactive |
| 31 | Inactive |
| 23 | Inactive |
| 22 | C |
| 37 | Inactive |
| 43 | Inactive |
| 39 | Inactive |
| 26 | Inactive |
| 38 | Inactive |
| 25 | Inactive |
| 21 | Inactive |
| 3 | Inactive |
| 2 | C |
| 6 | Inactive |
| 52 | Inactive |
| 5 | Inactive |
| Lenalidomide | 0.433 |
| Pomalidomide | 0.061 |
| CC-122 | 0.072 |
| CC-220 | 0.004 |
| CC-90009 | 0.189 |

Inactive represents IC50 > 50 μM, C represents 50 μM ≥ IC50 > 5 μM

### Example 49: Cell Viability, Additional Cell Lines

Tumor cells are grown at 37° C. in a humidified atmosphere with 5% CO2 in RPMI 1640 medium, supplemented with 10% (v/v) fetal calf serum and 50 μg/ml gentamicin for up to 20 passages, and are passaged once or twice weekly.

Cells are harvested from exponential phase cultures, counted and plated in 96 well flat-bottom microtiter plates at a cell density depending on the cell line's growth rate (4,000-20,000 cells/well depending on the cell line's growth rate, up to 60,000 for hematological cancer cell lines) in RPMI 1640 medium supplemented with 10% (v/v) fetal calf serum and 50 μg/ml gentamicin (140 μl/well). Cultures are incubated at 37° C. and 5% CO2 in a humidified atmosphere. After 24 h, 10 μl of test compounds or control medium are added and left on the cells for another 72 h. Compounds are serially diluted in DMSO, transferred in cell culture medium, and added to the assay plates by using a Tecan Freedom EVO 200 robotic platform. The DMSO concentration is kept constant at 0.3% v/v across the assay plate. Viability of cells is quantified by the CellTiter-Glo® cell viability assay (Promega G8462). After incubation of cells, 100 μl of CellTiter-Glo® One Solution Assay reagent are added to each well. Plates are shaken for 2 minutes to induce cell lysis and incubated for 20 minutes prior to reading luminescence (LU) by using the EnVision® Xcite multilabel plate reader (Perkin Elmer).

For single agent efficacy evaluation, sigmoidal concentration-response curves are fitted to the data points (test-versus-control, T/C values) obtained for each tumor model using 4 parameter non-linear curve fit (Charles River DRS Datawarehouse Software). IC50 values are reported as absolute IC50 values, being the concentration of test compound at the intersection of the concentration-response curves with T/C=50%. For calculation of mean IC50 values the geometric mean is used. Results are presented as heat maps (individual IC50 values relative to the geometric mean IC50 value) over all tumor models as tested.

TABLE 11
Cell lines sensitive to 1

| | Test/Control | |
|---|---|---|
| Cell line | viability at 9.5 μM* | Absolute IC50** |
| HEP-3B | A | B |
| KG-1 | A | A |
| KG-1a | A | A |
| UOC-M1 | A | A |
| 22Rv1 | B | C |
| MOLT-3 | B | C |
| MOLT-4 | B | C |
| MOLM-13 | B | C |
| MOLM-1 | A | A |
| MOLM-6 | B | B |
| MOLP-2 | B | B |

*Test/Control viability: A represents Test/Control viability ≤ 30%, B ≤ 60%
**Absolute IC50: A represents IC50 ≤ 100 nM, B - 100 nM < IC50 ≤ 1 μM, C - IC50 ≥ 30 μM

TABLE 12
Cell lines resistant to 1, but sensitive to CC-90009

Absolute IC50, μM

| Cell line | 1 | CC-90009 |
|---|---|---|
| EOL-1 | >30 | 0.002 |
| HL-60 | >30 | 0.003 |
| Kasumi-1 | >30 | 0.035 |
| NOMO-1 | >30 | 0.04 |
| OCI-AML2 | >30 | 0.018 |
| PL-21 | >30 | 0.01 |
| SU-DHL-1 | >30 | 0.153 |
| TMD8 | >30 | 0.264 |
| U-937 | >30 | 0.078 |
| IM-9 | >30 | 0.085 |
| LP-1 | >30 | 0.028 |
| NCI-H929 | >30 | 0.399 |
| OPM-2 | >30 | 0.074 |
| U-266 | >30 | 0.056 |

The compounds of the present invention potently inhibit growth of several cancer types: hepatocellular carcinoma (HEP3B, SNU-398), neuroblastoma (Kelly), leukemia (KG-1, KG-1a, UOC-M1, MOLT-3, MOLT-4, MOLM-13, MOLM-1, MOLM-6) prostate cancer (22Rv1), multiple myeloma (MOLP-2).

Simultaneously, the compounds of the present invention do not exhibit activity towards H929 and other cell lines listed in the table “Cell lines resistant to 1, but sensitive to CC-90009” showing differentiation with the prior art compounds, as exemplified by a clinical-stage compound CC-90009. This surprising effect corresponds to clinical attractiveness of the compounds, due to their enhanced selectivity that will likely correspond to a therapeutic window in particular cancer types, such as HCC.

### Example 50: Cell Survival

The effect of various compounds of the invention and various reference compounds on cell survival in Kelly and Hep3B cell lines was investigated, using the Cell Survival—Clonogenic Assay Protocol.

To determine the ability of a single cell to form a colony (defined as a cluster of at least 50 cells), Kelly and Hep3B cells were maintained in RPM11640 (Kelly) or EMEM (Hep3B) medium, supplemented with penicillin/streptomycin and 10% FBS. The cells were counted and seeded on the 6-well plates, at the density of 1×10³ cells per well, the compounds to be tested were added at the desired concentration range, and the cells were cultured at 37° C./5% CO₂. After colony formation (9-10 days), the cells were washed and treated with 6.0% glutaraldehyde and 0.5% crystal violet mix for 30 min., followed by rinsing with water and drying at room temperature (RT).

The compounds tested in this assay were Lenalidomide and 1 at the range of concentrations 0.1-10 μM. The crystal violet staining was performed after 9-10 days of culture. The results are shown in FIG. 8. As shown in this Figure, in the SALL4 expressing Kelly and Hep3B cell lines, the survival of the cells was in most cases inhibited by the compounds of the present invention, while lenalidomide or other market-known compounds presented no activity.

### ADDITIONAL DESCRIPTION

Also described herein are compounds for use in accordance with the following clauses:

Clause 1. A compound for use in a method of treating cancer, the method comprising administering the compound to a subject in need thereof, wherein the compound is:
(i) a compound of formula (I) or a pharmaceutically acceptable salt, ester, optically active isomer, racemate, solvate, amino acid conjugate, or prodrug thereof:

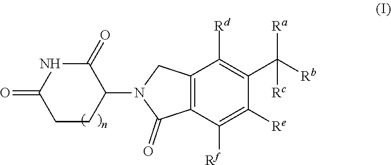

wherein
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
each of R^{d}, R^{e} and R^{f} is independently selected from H, deuterium, X, C₁-C₄ alkyl, and NH₂;
n is 1 or 2;
X is selected from F, Cl, Br and I;
R¹ is selected from H and C₁-C₄ alkyl,
R² is selected from H, C₁-C₄ alkyl, and —COR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is unsubstituted C₁-C₄ alkyl; or C₁-C₁₀ alkyl substituted with one or more R⁴, wherein each R⁴ is independently selected from NH₂, NHCOR⁵, NHCOOR⁵, OR⁵, unsubstituted 5-membered heterocyclyl, unsubstituted 6-membered heterocyclyl, 5-membered heteroaryl, and 6-membered heteroaryl;
R⁶ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl; and
R⁶ is unsubstituted cyclopentyl or cyclohexyl; or cyclopentyl or cyclohexyl substituted with one or more NH₂;
wherein, when R^{a}, R^{b}, R¹ and R² are each H, then n is 1;
or
(ii) a compound of formula (II) or a pharmaceutically acceptable salt, ester, optically active isomer, racemate, solvate, amino acid conjugate, or prodrug thereof:

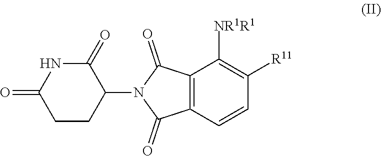

wherein:
each R¹ is independently selected from H and C₁-C₄ alkyl;
R¹¹ is OH or OR⁵; and
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

Clause 2. A pharmaceutical composition for use in a method of treating cancer, the method comprising administering the pharmaceutical composition to a subject in need thereof, wherein the pharmaceutical composition comprises:
(i) a compound of formula (I) or a pharmaceutically acceptable salt, ester, optically active isomer, racemate, solvate, amino acid conjugate, or prodrug thereof:

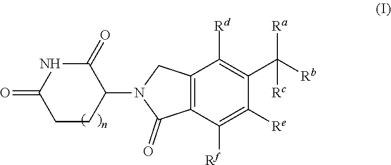

wherein
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
each of R^{d}, R*and R^{f} is independently selected from H, deuterium, X, C₁-C₄ alkyl, and NH₂;
n is 1 or 2;
X is selected from F, Cl, Br and I;
R¹ is selected from H and C₁-C₄ alkyl,
R² is selected from H, C₁-C₄ alkyl, and —COR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is unsubstituted C₁-C₄ alkyl; or C₁-C₁₀ alkyl substituted with one or more R⁴, wherein each R⁴ is independently selected from NH₂, NHCOR⁵, NHCOOR⁵, OR⁵, unsubstituted 5-membered heterocyclyl, unsubstituted 6-membered heterocyclyl, 5-membered heteroaryl, and 6-membered heteroaryl;
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl; and
R⁶ is unsubstituted cyclopentyl or cyclohexyl; or cyclopentyl or cyclohexyl substituted with one or more NH₂;
wherein, when R^{a}, R^{b}, R¹ and R² are each H, then n is 1;
or
(ii) a compound of formula (II) or a pharmaceutically acceptable salt, ester, optically active isomer, racemate, solvate, amino acid conjugate, or prodrug thereof:

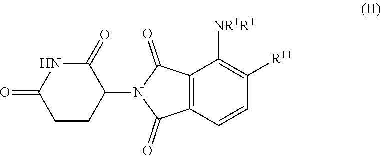

wherein:
each R¹ is independently selected from H and C₁-C₄ alkyl;
R¹¹ is OH or OR⁵;
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

Clause 3. The compound or composition for use of Clause 1 or Clause 2, wherein the compound of formula (I) is
a compound of formula (Ia) or a pharmaceutically acceptable salt, ester, optically active isomer, racemate, solvate, amino acid conjugate, or prodrug thereof:

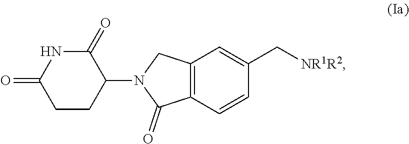

wherein;
R¹ is selected from H and C₁-C₄ alkyl;
R² is selected from H and —COR³;
R³ is unsubstituted C₁-C₄ alkyl; or C₁-C₁₀ alkyl substituted with one or more R⁴, wherein each R⁴ is independently selected from NH₂, NHCOR⁵, NHCOOR⁵, OR⁵, unsubstituted 5-membered heterocyclyl, unsubstituted 6-membered heterocyclyl, 5-membered heteroaryl, and 6-membered heteroaryl; and
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

Clause 4. The compound or composition for use of any preceding Clause, wherein R¹¹ is OH.

Clause 5. The compound or composition for use of any preceding Clause, wherein NR¹R¹ is NH₂.

Clause 6. The compound or composition for use of any preceding Clause, wherein the cancer is hepatocellular carcinoma, neuroblastoma, acute myeloid leukemia (AML), acute promyelocytic leukemia (APL), breast cancer, prostate cancer, bladder cancer, kidney cancer, muscle cancer, ovary cancer, skin cancer, pancreas cancer, breast cancer, colon cancer, hematological cancer, cancer of a connective tissue, placenta cancer, bone cancer, uterus cancer, cervical cancer, choriocarcinoma, endometrial cancer, gastric cancer, or lung cancer.

Clause 7. The compound or composition for use of any preceding Clause, wherein the cancer is hepatocellular carcinoma.

Clause 8. The compound or composition for use of any one of Clauses 1-6, wherein the cancer is neuroblastoma.

Clause 9. The compound or composition for use of any preceding Clause, wherein the method of treating cancer further comprises administering a second cancer therapy to the subject.

Clause 10. The compound or composition for use of Clause 9, wherein the second cancer therapy is chemotherapy or radiotherapy.

Clause 11. The compound or composition for use of Clause 9 or Clause 10, wherein:
(a) the cancer is chronic myeloid leukemia (CML) and the second cancer therapy is chemotherapy with imatinib, dasatinib or nilotinib;
(b) the cancer is endometrial cancer and the second cancer therapy is chemotherapy with carboplatin;
(c) the cancer is glioblastoma and the second cancer therapy is chemotherapy with temozolomide (TMZ);
(d) the cancer is lung cancer and the second cancer therapy is chemotherapy with cisplatin;
(e) the cancer is lung cancer and the second cancer therapy is chemotherapy with Erlotinib;
(f) the cancer is lung cancer and the second cancer therapy is chemotherapy with entinostat;
(g) the cancer is lung cancer and the second cancer therapy is chemotherapy with cisplatin, carboplatin or paclitaxel;
(h) the cancer is myelodysplastic syndrome (MDS)/acute myeloid leukemia (AML) and the second cancer therapy is chemotherapy with doxorubicin; or
(i) the cancer is nasopharyngeal carcinoma and the second cancer therapy is radiotherapy.

Clause 12. The compound or composition for use of any preceding Clause, wherein the compound is selected from

| Compound ID | Structure |
|---|---|
| 1 | 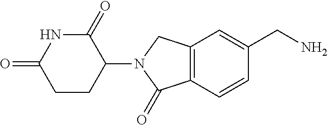 [SMILES: NCc1ccc2c(c1)CN(C1CCC(=O)NC1=O)C2=O] |
| 12 | 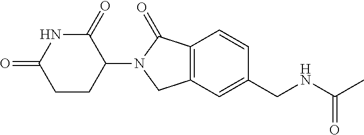 [SMILES: CC(=O)NCc1ccc2c(c1)CN(C1CCC(=O)NC1=O)C2=O] |
| 28 | 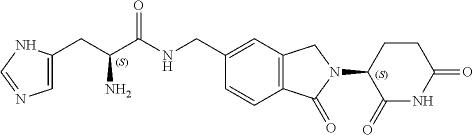 |
| 14 | 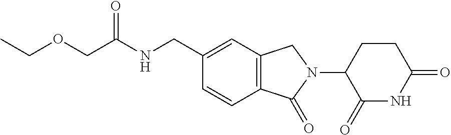 [SMILES: CCOCC(=O)NCc1ccc2c(c1)CN(C1CCC(=O)NC1=O)C2=O] |
| 27 | 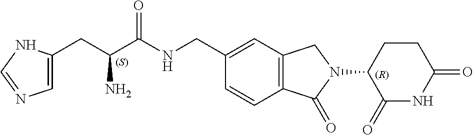 |
| 30 | 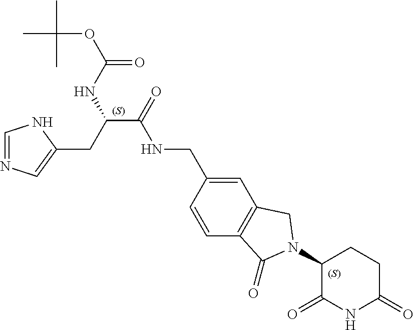 |
| 44 | 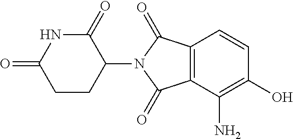 [SMILES: Nc1c(O)ccc2c1C(=O)N(C1CCC(=O)NC1=O)C2=O] |
| 29 | 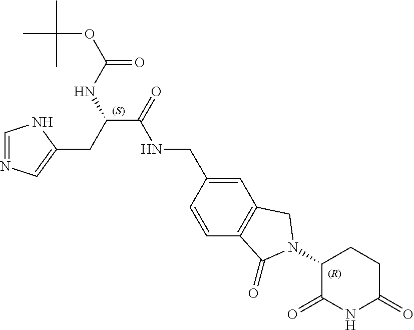 |
| 24 | 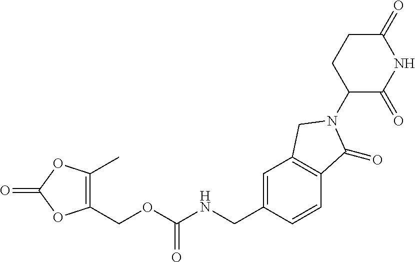 [SMILES: Cc1oc(=O)oc1COC(=O)NCc1ccc2c(c1)CN(C1CCC(=O)NC1=O)C2=O] |

and pharmaceutically acceptable salts, esters, optically active isomers, racemates, solvates, amino acid conjugates, or prodrugs thereof.

Clause 13. The compound or composition for use of Clause 12, wherein the compound:
(a) is selected from compounds 12, 14, 30 and 29,
(b) is selected from compounds 1, 28, 27 and 24,
(c) is selected from compounds 1, 44, 28, 27, and 24,
(d) is selected from compounds 44, 28, 27 and 24,
(e) is selected from compounds 1, 44, 28 and 27,
(f) is selected from compounds 44 and 1, or
(g) is compound 1.

Clause 14. A compound for use in a method of modulating levels of a target protein in a subject, the method comprising administering the compound to the subject, wherein the compound is a compound of formula (III) or a pharmaceutically acceptable salt, ester, optically active isomer, racemate, solvate, amino acid conjugate, or prodrug thereof:

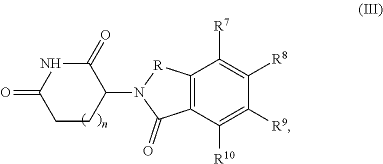

wherein;
R is C═O or CH₂;
R⁷ is selected from H, deuterium, X, C₁-C₄ alkyl, and NR¹R¹,
R⁸ is selected from OH, OR⁵, and CR^{a}R^{b}R^{c},
wherein
   when R is C═O and R⁸ is OH, then R⁷ is selected from NR¹R¹, deuterium, X, and C₁-C₄ alkyl;
R⁹ and R¹⁰ are independently selected from H, deuterium, X, C₁-C₄ alkyl, and NH₂;
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
n is 1 or 2;
X is selected from F, Cl, Br and I;
R¹ is selected from H and C₁-C₄ alkyl,
R² is selected from H, C₁-C₄ alkyl, and —COR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or when R⁸ is CR^{a}R^{b}R^{c} and R^{c} is NR¹R² then R¹ and R^{a}, together with the carbon and nitrogen atoms to which they are attached, form a 5 or 6-membered heterocycle;
R³ is unsubstituted C₁-C₁₀ alkyl; or C₁-C₁₀ alkyl substituted with one or more R⁴, wherein each R⁴ is independently selected from NH₂, NHCOR⁵, NHCOOR⁵, OR⁵, unsubstituted 5-membered heterocyclyl, unsubstituted 6-membered heterocyclyl, 5-membered heteroaryl, and 6-membered heteroaryl;
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl; and
R⁶ is unsubstituted cyclopentyl or cyclohexyl; or cyclopentyl or cyclohexyl substituted with one or more NH₂.

Clause 15. A pharmaceutical composition for use in a method of modulating levels of a target protein in a subject, the method comprising administering the pharmaceutical composition to the subject, wherein the pharmaceutical composition comprises a compound of formula (III) or a pharmaceutically acceptable salt, ester, optically active isomer, racemate, solvate, amino acid conjugate, or prodrug thereof:

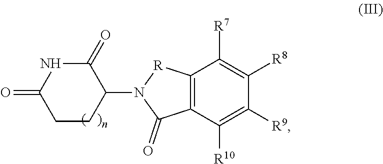

wherein;
R is C═O or CH₂;
R⁷ is selected from H, deuterium, X, C₁-C₄ alkyl, and NR¹R¹,
R⁸ is selected from OH, OR⁵, and CR^{a}R^{b}R^{c},
wherein
   when R is C═O and R⁸ is OH, then R⁷ is selected from NR¹R¹, deuterium, X, and C₁-C₄ alkyl;
R⁹ and R¹⁰ are independently selected from H, deuterium, X, C₁-C₄ alkyl, and NH₂;
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
n is 1 or 2;
X is selected from F, Cl, Br and I;
R¹ is selected from H and C₁-C₄ alkyl,
R² is selected from H, C₁-C₄ alkyl, and —COR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or when R⁸ is CR^{a}R^{b}R^{c} and R^{c} is NR¹R² then R¹ and R^{a}, together with the carbon and nitrogen atoms to which they are attached, form a 5 or 6-membered heterocycle;
R³ is unsubstituted C₁-C₁₀ alkyl; or C₁-C₁₀ alkyl substituted with one or more R⁴, wherein each R⁴ is independently selected from NH₂, NHCOR⁵, NHCOOR⁵, OR⁵, unsubstituted 5-membered heterocyclyl, unsubstituted 6-membered heterocyclyl, 5-membered heteroaryl, and 6-membered heteroaryl;
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl; and
R⁶ is unsubstituted cyclopentyl or cyclohexyl; or cyclopentyl or cyclohexyl substituted with one or more NH₂.

Clause 16. An in vitro method of modulating levels of a target protein in cells, comprising administering to the cells a compound of formula (III) or a pharmaceutically acceptable salt, ester, optically active isomer, racemate, solvate, amino acid conjugate, or prodrug thereof:

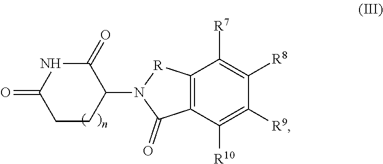

wherein;
R is C═O or CH₂;
R⁷ is selected from H, deuterium, X, C₁-C₄ alkyl, and NR¹R¹,
R⁸ is selected from OH, OR⁵, and CR^{a}R^{b}R^{c},
wherein
   when R is C═O and R⁸ is OH, then R⁷ is selected from NR¹R¹, deuterium, X, and C₁-C₄ alkyl;
R⁹ and R¹⁰ are independently selected from H, deuterium, X, C₁-C₄ alkyl, and NH₂;
R^{a} is selected from H, deuterium, and C₁-C₄ alkyl;
R^{b} is selected from H, deuterium, and C₁-C₄ alkyl;
R^{c} is selected from NR¹R², OH, OR⁶, CH₂X, CHX₂, and CX₃;
n is 1 or 2;
X is selected from F, Cl, Br and I;
R¹ is selected from H and C₁-C₄ alkyl,
R² is selected from H, C₁-C₄ alkyl, and —COR³,
or alternatively R¹ and R², together with the nitrogen atom to which they are attached, form an 5 or 6-membered heterocycle, wherein the heterocycle is unsubstituted or wherein one or more carbon atoms of the heterocycle form part of a carbonyl group; or when R⁸ is CR^{a}R^{b}R^{c} and R^{c} is NR¹R² then R¹ and R^{a}, together with the carbon and nitrogen atoms to which they are attached, form a 5 or 6-membered heterocycle;
R³ is unsubstituted C₁-C₁₀ alkyl; or C₁-C₁₀ alkyl substituted with one or more R⁴, wherein each R⁴ is independently selected from NH₂, NHCOR⁵, NHCOOR⁵, OR⁵, unsubstituted 5-membered heterocyclyl, unsubstituted 6-membered heterocyclyl, 5-membered heteroaryl, and 6-membered heteroaryl;
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl; and
R⁶ is unsubstituted cyclopentyl or cyclohexyl; or cyclopentyl or cyclohexyl substituted with one or more NH₂.

Clause 17. The compound or composition for use of Clause 14 or Clause 15, or the method of clause 16, wherein the target protein is SALL4.

Clause 18. The compound for use, composition for use, or method of any one of Clauses 14-17 wherein, when R^{a}, R^{b}, R¹ and R² are each H, then n is 1.

Clause 19. The compound for use, composition for use, or method of any one of Clauses 14-18, wherein the compound is a compound of formula (IIIa) or a pharmaceutically acceptable salt, ester, optically active isomer, racemate, solvate, amino acid conjugate, or prodrug thereof:

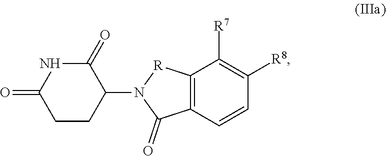

wherein;
R is C═O or CH₂;
R⁷ is selected from H and NR¹R¹,
R⁸ is selected from OH, OR⁶, and CH₂NR¹R²,
wherein
   when R is C═O and R⁸ is OH, then R⁷ is NR¹R¹;
R¹ is H or C₁-C₄ alkyl;
R² is H or —COR³;
R³ is unsubstituted C₁-C₁₀ alkyl; or C₁-C₁₀ alkyl substituted with one or more R⁴, wherein each R⁴ is independently selected from NH₂, NHCOR⁵, NHCOOR⁵, OR⁵, 5-membered heterocyclyl, 6-membered heterocyclyl, 5-membered heteroaryl, and 6-membered heteroaryl; and
R⁵ is unsubstituted C₁-C₆ alkyl; or C₁-C₆ alkyl substituted with one or more substituents independently selected from 5-membered heterocyclyl, 6-membered heterocyclyl, 5-membered heteroaryl and 6-membered heteroaryl.

Clause 20. The compound for use, composition for use, or method of any one of Clauses 14-19, wherein R is CH₂.

Clause 21. The compound for use, composition for use, or method of Clause 20, wherein R⁷ is H, and R⁸ is CH₂NR¹R².

Clause 22. The compound or composition for use, method of any one of Clauses 14-19, wherein R is C═O.

Clause 23. The compound for use, composition for use, or method of Clause 22, wherein R⁷ is NR¹R¹, and R⁸ is OH or OR⁵.

Clause 24. The compound for use, composition for use, or method of any preceding Clause, wherein R¹ is H.

Clause 25. The compound for use, composition for use, or method of any preceding Clause, wherein R² is —COR³.

Clause 26. The compound for use, composition for use, or method of any preceding Clause, wherein R³ is unsubstituted C₁-C₁₀ alkyl.

Clause 27. The compound for use, composition for use, or method of any one of Clauses 1-25, wherein R³ is C₁-C₁₀ alkyl substituted with one or more R⁴, wherein each R⁴ is independently selected from NH₂, NHCOR⁵, NHCOOR⁵, OR⁵, 5-membered heterocyclyl, 6-membered heterocyclyl, 5-membered heteroaryl, and 6-membered heteroaryl.

Clause 28. The compound for use, composition for use, or method of Clause 27, wherein R³ is C₁-C₁₀ alkyl substituted with one or more R⁴, wherein each R⁴ is independently selected from NH₂, NHCOR⁵ and NHCOOR⁵.

Clause 29. The compound for use, composition for use, or method of any one of Clauses 14-17, wherein the compound is selected from

| Compound ID | Structure |
|---|---|
| 1 | 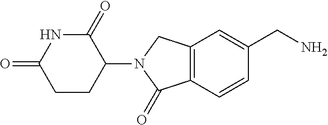 [SMILES: NCc1ccc2c(c1)CN(C1CCC(=O)NC1=O)C2=O] |
| 12 | 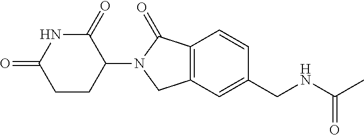 [SMILES: CC(=O)NCc1ccc2c(c1)CN(C1CCC(=O)NC1=O)C2=O] |
| 28 | 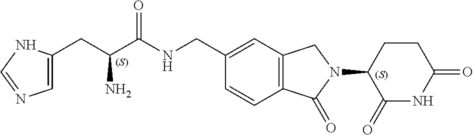 |
| 14 | 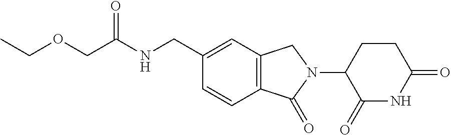 [SMILES: CCOCC(=O)NCc1ccc2c(c1)CN(C1CCC(=O)NC1=O)C2=O] |
| 27 | 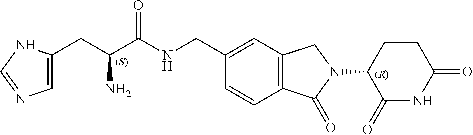 |
| 30 | 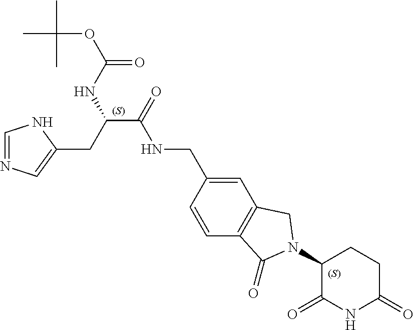 |
| 29 | 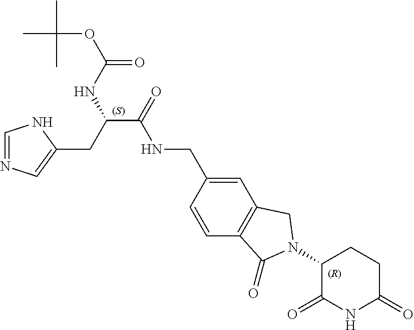 |
| 44 | 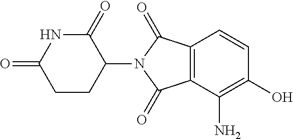 [SMILES: Nc1c(O)ccc2c1C(=O)N(C1CCC(=O)NC1=O)C2=O] |
| 24 | 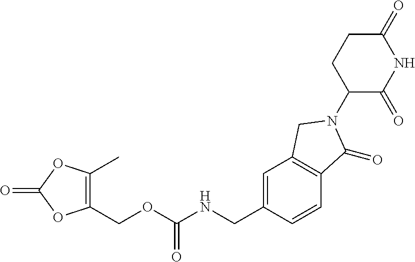 [SMILES: Cc1oc(=O)oc1COC(=O)NCc1ccc2c(c1)CN(C1CCC(=O)NC1=O)C2=O] |

and pharmaceutically acceptable salts, esters, optically active isomers, racemates, solvates, amino acid conjugates, or prodrugs thereof.

Clause 30. The compound for use, composition for use, or method of Clause 29, wherein the compound:
(a) is selected from compounds 12, 14, 44, 30 and 29,
(b) is selected from compounds 1, 28, 27 and 24,
(c) is selected from compounds 1, 44, 28, 27, and 24,
(d) is selected from compounds 44, 28, 27 and 24,
(e) is selected from compounds 1, 44, 28 and 27,
(f) is compound 1.

1. A compound of formula (Ia), (Ib) or (Ic): 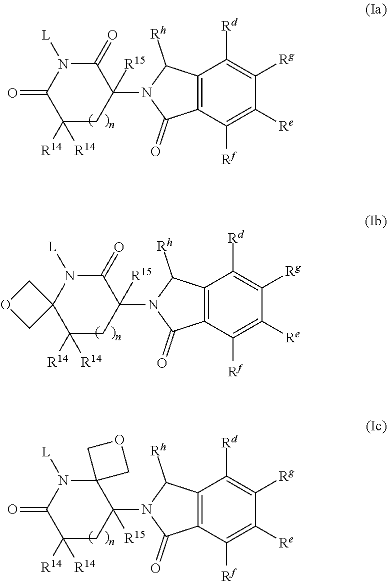
or a pharmaceutically acceptable salt, ester, optically active isomer, racemate, solvate, amino acid conjugate, or prodrug thereof, wherein
L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″ or P(O)(OR″)(OR″);
each R″ is independently selected from hydrogen, alkyl, cycloalkyl, alkenyl, cycloalkenyl, aryl, heteroaryl, or benzyl;
each R¹⁴ is independently selected from deuterium or hydrogen;
R¹⁵ is selected from hydrogen, deuterium or C₁-C₄ alkyl;
R^{g} is CR^{a}R^{b}R^{c},
R^{h} is selected from H or C₁-C₄ alkyl;
R^{a} is selected from H, deuterium, or C₁-C₄ alkyl;
R^{b} is selected from H, deuterium, or C₁-C₄ alkyl;
R^{c} is selected from NR¹R², OH, OR⁶, CH₂X, CHX₂, or CX₃;
each of R^{d}, R^{e} and R^{f} is independently selected from H, deuterium, X, C₁-C₄ alkyl, or NH₂;
n is 0, 1 or 2;
X is selected from F, Cl, Br or I;
R¹ is selected from H or C₁-C₃ alkyl;
R² is selected from H, C₁-C₃ alkyl, —COR³, or —COOR³;
or R¹ and R², together with the nitrogen atom to which they are attached, form a 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is selected from:
unsubstituted C₁-C₄ alkyl;
C₁-C₁₀ alkyl substituted with one or more R⁴, wherein each R⁴ is independently selected from —NH₂, —NHC(NH)NH₂, —NHCOR⁵, —NHCOOR⁵, —OH, —OR⁵, —OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, and 6-membered aryl substituted with one or more substituents independently selected from C₁-C₄ alkyl, —OH, —CH₂—OH, —OCO(C₁-C₄ alkyl) or CH₂OCO(C₁-C₄ alkyl); and wherein R⁴ is not X;
C₂-C₁₀ alkyl substituted with a halophenyl group;
6-membered aryl substituted with one or more substituents independently selected from CH₂—OH or CH₂OCO(C₁-C₄ alkyl);
unsubstituted 5- or 6-membered heterocyclyl;
R⁵ is unsubstituted C₁-C₆ alkyl; or C₁-C₆ alkyl substituted with one or more substituents independently selected from 5-membered heterocyclyl, 6-membered heterocyclyl, 5-membered aryl, 6-membered aryl, 5-membered heteroaryl or 6-membered heteroaryl; and
R⁶ is unsubstituted cyclopentyl or cyclohexyl; or cyclopentyl or cyclohexyl substituted with one or more NH₂;
wherein, in formula (Ia):
when R^{a}, R^{b}, R¹ and R² are each H, then n is 0 or 1;
when R^{a}, R^{b}, R^{d}, R^{e}, R^{f}, R^{h}, R¹, R², R¹⁴ and L are each H, and R¹⁵ is H or C₁-C₄ alkyl, then n is 0;
when R^{a}, R^{b} and R¹ are each H and R² is —COR³, then n is 0 or 1; and
when R^{a}, R^{b}, R^{d}, R^{e}, R^{f} and R¹ are each H and R³ is unsubstituted C₁-C₄ alkyl, then n is 0.

2. A compound of formula (II): 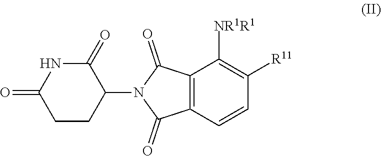
or a pharmaceutically acceptable salt, ester, optically active isomer, racemate, solvate, amino acid conjugate, or prodrug thereof,
wherein:
each R¹ is independently selected from H or C₁-C₄ alkyl;
R¹¹ is OH or OR^{5a}; and
R^{5a} is unsubstituted C₁-C₆ alkyl; or C₁-C₆ alkyl substituted with one or more substituents independently selected from 5-membered heterocyclyl, 6-membered heterocyclyl, 5-membered heteroaryl or 6-membered heteroaryl.

3. The compound of claim 1, wherein R³ is selected from:
unsubstituted C₁-C₄ alkyl;
C₁-C₁₀ alkyl substituted with one or more R⁴, wherein each R⁴ is independently selected from —NH₂, —NHC(NH)NH₂, —NHCOR⁵, —NHCOOR⁵, —OH, —OR⁵, —OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, or 6-membered aryl substituted with one or more substituents independently selected from C₁-C₄ alkyl, —OH, —CH₂—OH, —OCO(C₁-C₄ alkyl) or —CH₂OCO(C₁-C₄ alkyl); and wherein R⁴ is not X;
6-membered aryl substituted with one or more substituents independently selected from CH₂—OH or CH₂OCO(C₁-C₄ alkyl); and
unsubstituted 5- or 6-membered heterocyclyl.

4. The compound of claim 1, wherein R³ is selected from:
unsubstituted C₁-C₄ alkyl;
C₁-C₁₀ alkyl substituted with one or more R⁴, wherein each R⁴ is independently selected from —NH₂, —NHC(NH)NH₂, —NHCOR⁵, —NHCOOR⁵, —OH, —OR⁵, —OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, or 6-membered aryl substituted with one or more substituents independently selected from C₁-C₄ alkyl, —OH, —CH₂—OH, —OCO(C₁-C₄ alkyl) or —CH₂OCO(C₁-C₄ alkyl); wherein R⁴ is not X, and wherein when the C₁-C₁₀ alkyl is substituted with indole, the C₁-C₁₀ alkyl is further substituted with at least one additional R⁴;
6-membered aryl substituted with one or more substituents independently selected from CH₂—OH or CH₂OCO(C₁-C₄ alkyl); and
unsubstituted 5- or 6-membered heterocyclyl.

5. The compound of claim 2, wherein R¹¹ is OH.

6. The compound of claim 1, wherein NR¹R¹ is NH₂.

7. The compound of claim 1, wherein R^{h} is H

8. The compound of claim 1, wherein R^{h} is methyl.

9. The compound of claim 1, wherein R^{a} and R^{b} are each H.

10. The compound of claim 1, wherein R^{a} and R^{b} are each deuterium.

11. The compound of claim 1, wherein R^{a} is H and R^{b} is methyl.

12. The compound of claim 1, wherein R^{c} is selected from NHR² and OH.

13. The compound of claim 1, wherein the compound is selected from:

| Compound | |
|---|---|
| ID | Structure |
| 2 | 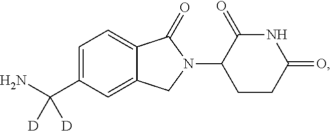 [SMILES: [2H]C([2H])(N)c1ccc2c(c1)CN(C1CCC(=O)NC1=O)C2=O] |
| 3 | 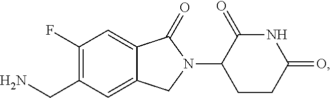 [SMILES: NCc1cc2c(cc1F)C(=O)N(C1CCC(=O)NC1=O)C2] |
| 4 | 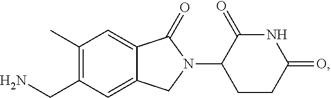 [SMILES: Cc1cc2c(cc1CN)CN(C1CCC(=O)NC1=O)C2=O] |
| 5 | 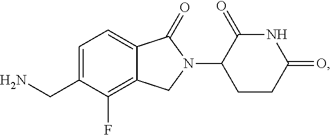 [SMILES: NCc1ccc2c(c1F)CN(C1CCC(=O)NC1=O)C2=O] |
| 6 | 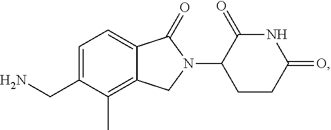 [SMILES: Cc1c(CN)ccc2c1CN(C1CCC(=O)NC1=O)C2=O] |
| 7 | 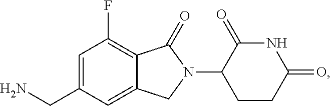 [SMILES: NCc1cc(F)c2c(c1)CN(C1CCC(=O)NC1=O)C2=O] |
| 8 | 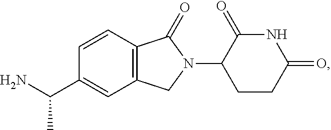 [SMILES: C[C@H](N)c1ccc2c(c1)CN(C1CCC(=O)NC1=O)C2=O] |
| 9 | 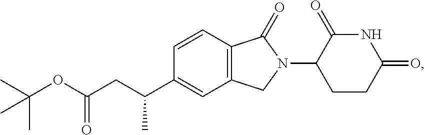 [SMILES: C[C@H](CC(=O)OC(C)(C)C)c1ccc2c(c1)CN(C1CCC(=O)NC1=O)C2=O] |
| 10 | 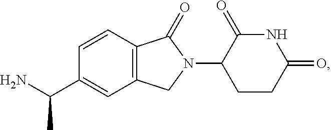 [SMILES: C[C@@H](N)c1ccc2c(c1)CN(C1CCC(=O)NC1=O)C2=O] |
| 11 | 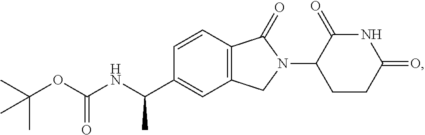 [SMILES: C[C@@H](NC(=O)OC(C)(C)C)c1ccc2c(c1)CN(C1CCC(=O)NC1=O)C2=O] |
| 13 | 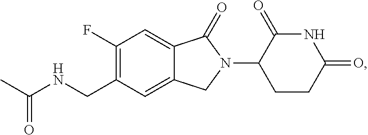 [SMILES: CC(=O)NCc1cc2c(cc1F)C(=O)N(C1CCC(=O)NC1=O)C2] |
| 14 | 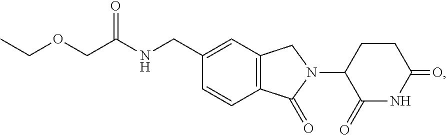 [SMILES: CCOCC(=O)NCc1ccc2c(c1)CN(C1CCC(=O)NC1=O)C2=O] |
| 16 | 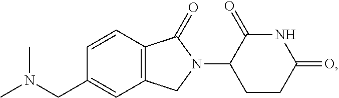 [SMILES: CN(C)Cc1ccc2c(c1)CN(C1CCC(=O)NC1=O)C2=O] |
| 17 | 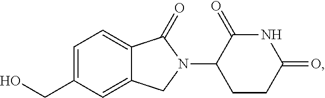 [SMILES: O=C1CCC(N2Cc3cc(CO)ccc3C2=O)C(=O)N1] |
| 18 | 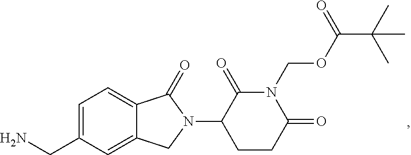 [SMILES: CC(C)(C)C(=O)OCN1C(=O)CCC(N2Cc3cc(CN)ccc3C2=O)C1=O] |
| 19 | 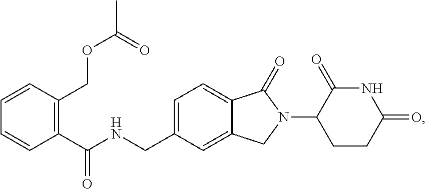 [SMILES: CC(=O)OCc1ccccc1C(=O)NCc1ccc2c(c1)CN(C1CCC(=O)NC1=O)C2=O] |
| 20 | 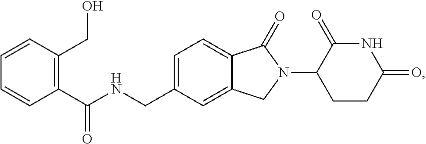 [SMILES: O=C1CCC(N2Cc3cc(CNC(=O)c4ccccc4CO)ccc3C2=O)C(=O)N1] |
| 21 | 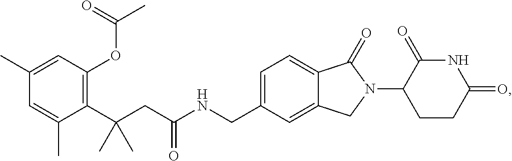 [SMILES: CC(=O)Oc1cc(C)cc(C)c1C(C)(C)CC(=O)NCc1ccc2c(c1)CN(C1CCC(=O)NC1=O)C2=O] |
| 22 | 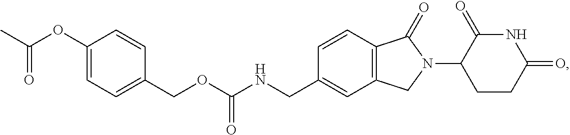 [SMILES: CC(=O)Oc1ccc(COC(=O)NCc2ccc3c(c2)CN(C2CCC(=O)NC2=O)C3=O)cc1] |
| 23 | 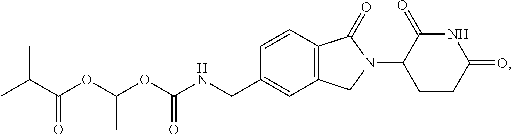 [SMILES: CC(OC(=O)NCc1ccc2c(c1)CN(C1CCC(=O)NC1=O)C2=O)OC(=O)C(C)C] |
| 24 | 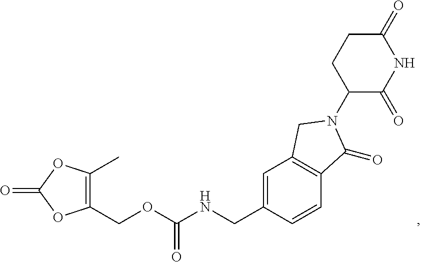 [SMILES: Cc1oc(=O)oc1COC(=O)NCc1ccc2c(c1)CN(C1CCC(=O)NC1=O)C2=O] |
| 25 | 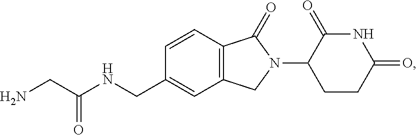 [SMILES: NCC(=O)NCc1ccc2c(c1)CN(C1CCC(=O)NC1=O)C2=O] |
| 26 | 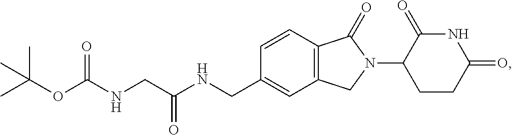 [SMILES: CC(C)(C)OC(=O)NCC(=O)NCc1ccc2c(c1)CN(C1CCC(=O)NC1=O)C2=O] |
| 27 | 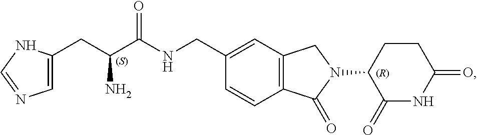 |
| 28 | 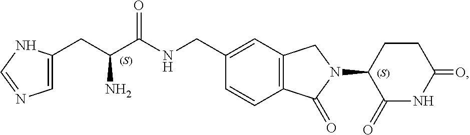 |
| 29 | 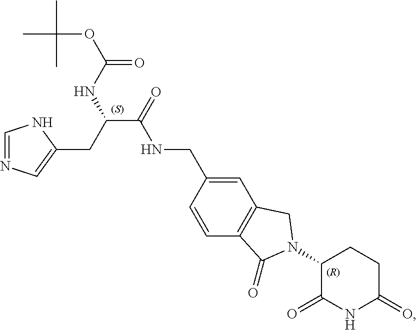 |
| 30 | 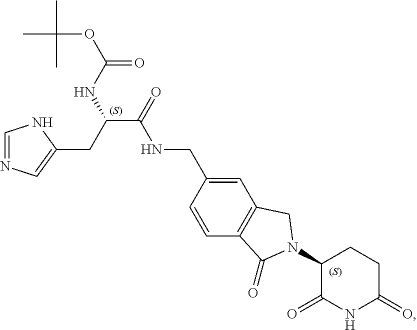 |
| 31 | 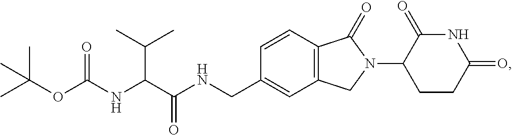 [SMILES: CC(C)C(NC(=O)OC(C)(C)C)C(=O)NCc1ccc2c(c1)CN(C1CCC(=O)NC1=O)C2=O] |
| 32 | 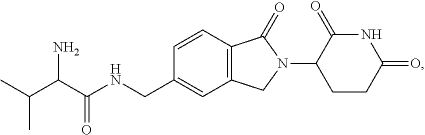 [SMILES: CC(C)C(N)C(=O)NCc1ccc2c(c1)CN(C1CCC(=O)NC1=O)C2=O] |
| 33 | 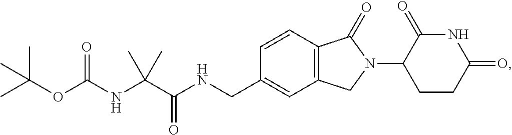 [SMILES: CC(C)(C)OC(=O)NC(C)(C)C(=O)NCc1ccc2c(c1)CN(C1CCC(=O)NC1=O)C2=O] |
| 34 | 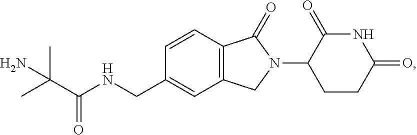 [SMILES: CC(C)(N)C(=O)NCc1ccc2c(c1)CN(C1CCC(=O)NC1=O)C2=O] |
| 35 | 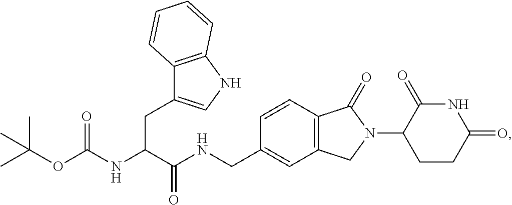 [SMILES: CC(C)(C)OC(=O)NC(Cc1c[nH]c2ccccc12)C(=O)NCc1ccc2c(c1)CN(C1CCC(=O)NC1=O)C2=O] |
| 37 | 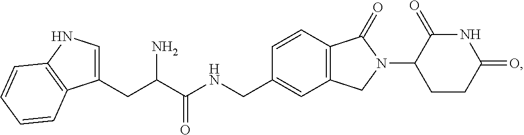 [SMILES: NC(Cc1c[nH]c2ccccc12)C(=O)NCc1ccc2c(c1)CN(C1CCC(=O)NC1=O)C2=O] |
| 38 | 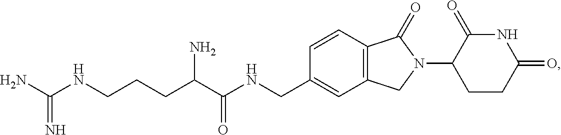 [SMILES: N=C(N)NCCCC(N)C(=O)NCc1ccc2c(c1)CN(C1CCC(=O)NC1=O)C2=O] |
| 39 | 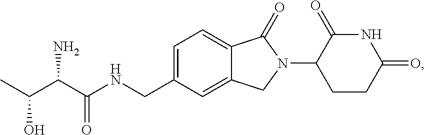 [SMILES: C[C@@H](O)[C@H](N)C(=O)NCc1ccc2c(c1)CN(C1CCC(=O)NC1=O)C2=O] |
| 40 | 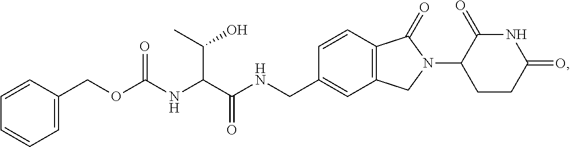 [SMILES: C[C@H](O)C(NC(=O)OCc1ccccc1)C(=O)NCc1ccc2c(c1)CN(C1CCC(=O)NC1=O)C2=O] |
| 41 | 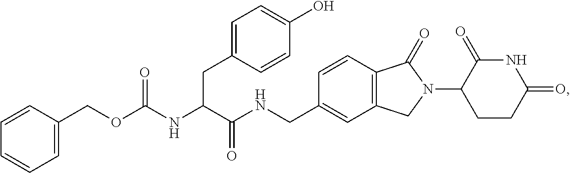 [SMILES: O=C1CCC(N2Cc3cc(CNC(=O)C(Cc4ccc(O)cc4)NC(=O)OCc4ccccc4)ccc3C2=O)C(=O)N1] |
| 43 | 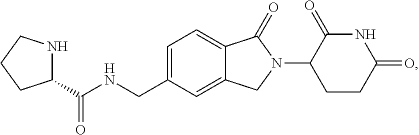 [SMILES: O=C1CCC(N2Cc3cc(CNC(=O)[C@@H]4CCCN4)ccc3C2=O)C(=O)N1] |
| 50 | 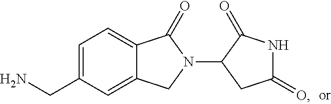 [SMILES: NCc1ccc2c(c1)CN(C1CC(=O)NC1=O)C2=O] |
| 52 | 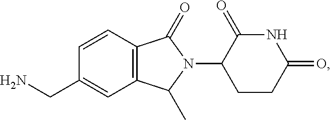 [SMILES: CC1c2cc(CN)ccc2C(=O)N1C1CCC(=O)NC1=O] |

or a pharmaceutically acceptable salt, ester, optically active isomer, racemate, solvate, amino acid conjugate, or prodrug thereof.

14. The compound of claim 1, wherein R^{c} is NHR².

15. The compound of claim 1, wherein R² is selected from H, —COR³, or —COOR³

16. The compound of claim 1, wherein R³ is C₁-C₁₀ alkyl substituted with one or more R⁴.

17. The compound of claim 1, wherein each R⁴ is independently selected from NH₂, OCOR⁵, substituted or unsubstituted dioxolyl, indole, or 6-membered aryl substituted with one or more —OCO(C₁-C₄ alkyl); wherein R⁴ is not X.

18. The compound of claim 1, wherein the compound is selected from:

| | |
|---|---|
| 51 | 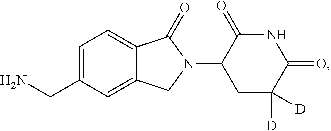 [SMILES: [2H]C1([2H])CC(N2Cc3cc(CN)ccc3C2=O)C(=O)NC1=O] |
| 6 | 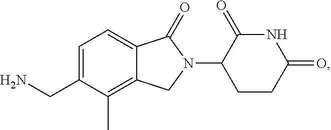 [SMILES: Cc1c(CN)ccc2c1CN(C1CCC(=O)NC1=O)C2=O] |
| 2 | 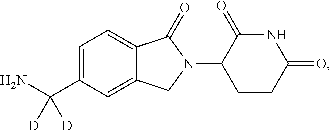 [SMILES: [2H]C([2H])(N)c1ccc2c(c1)CN(C1CCC(=O)NC1=O)C2=O] |
| 23 | 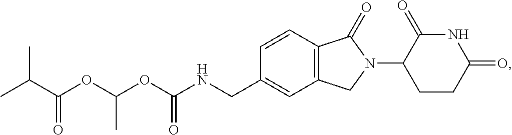 [SMILES: CC(OC(=O)NCc1ccc2c(c1)CN(C1CCC(=O)NC1=O)C2=O)OC(=O)C(C)C] |
| 22 | 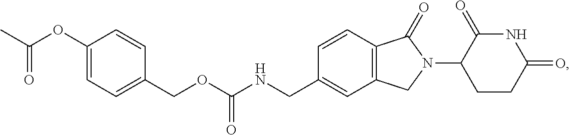 [SMILES: CC(=O)Oc1ccc(COC(=O)NCc2ccc3c(c2)CN(C2CCC(=O)NC2=O)C3=O)cc1] |
| 52 | 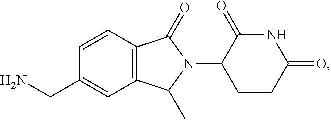 [SMILES: CC1c2cc(CN)ccc2C(=O)N1C1CCC(=O)NC1=O] |
| 3 | 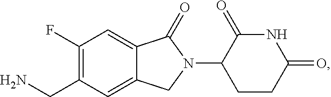 [SMILES: NCc1cc2c(cc1F)C(=O)N(C1CCC(=O)NC1=O)C2] |
| 37 | 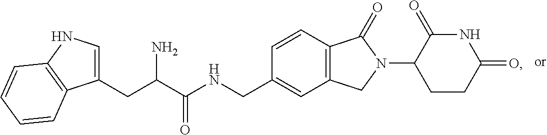 [SMILES: NC(Cc1c[nH]c2ccccc12)C(=O)NCc1ccc2c(c1)CN(C1CCC(=O)NC1=O)C2=O] |
| 24 | 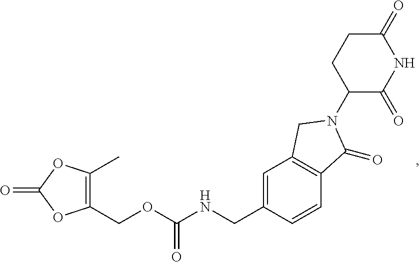 [SMILES: Cc1oc(=O)oc1COC(=O)NCc1ccc2c(c1)CN(C1CCC(=O)NC1=O)C2=O] |

or a pharmaceutically acceptable salt, ester, optically active isomer, racemate, solvate, amino acid conjugate, or prodrug thereof.

19. A pharmaceutical composition comprising a compound of claim 1.

20. A method of treating cancer comprising administering a compound to a subject in need thereof, wherein the compound is:
(i) a compound of formula (Ia), (Ib) or (Ic): 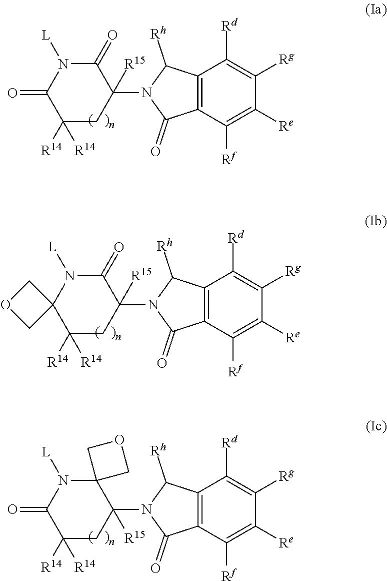
or a pharmaceutically acceptable salt, ester, optically active isomer, racemate, solvate, amino acid conjugate, or prodrug thereof,
wherein
L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″ or P(O)(OR″)(OR″);
each R″ is independently selected from hydrogen, alkyl, cycloalkyl, alkenyl, cycloalkenyl, aryl, heteroaryl, or benzyl;
each R¹⁴ is independently selected from deuterium or hydrogen;
R¹⁵ is selected from hydrogen, deuterium or C₁-C₄ alkyl;
R^{g} is selected from —COOH or —CR^{a}R^{b}R^{c},
R^{h} is selected from H or C₁-C₄ alkyl;
R^{a} is selected from H, deuterium, or C₁-C₄ alkyl;
R^{b} is selected from H, deuterium, or C₁-C₄ alkyl;
R^{c} is selected from NR¹R², OH, OR⁶, CH₂X, CHX₂, or CX₃;
each of R^{d}, R^{e} and R^{f} is independently selected from H, deuterium, X, C₁-C₄ alkyl, or NH₂;
n is 0, 1 or 2;
X is selected from F, Cl, Br or I;
R¹ is selected from H or C₁-C₄ alkyl,
R² is selected from H, C₁-C₄ alkyl, —COR³, or —COOR³,
or R¹ and R², together with the nitrogen atom to which they are attached, form a 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is selected from:
unsubstituted C₁-C₄ alkyl;
C₁-C₁₀ alkyl substituted with one or more R⁴, wherein each R⁴ is independently selected from —NH₂, —NHC(NH)NH₂, —NHCOR⁵, —NHCOOR⁵, —OH, —OR⁵, —OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, or 6-membered aryl substituted with one or more substituents independently selected from C₁-C₄ alkyl, —OH, —CH₂—OH, —OCO(C₁-C₄ alkyl) or —CH₂OCO(C₁-C₄ alkyl); and wherein R⁴ is not X;
C₂-C₁₀ alkyl substituted with a halophenyl group;
6-membered aryl substituted with one or more substituents independently selected from CH₂—OH or CH₂OCO(C₁-C₄ alkyl); and
unsubstituted 5- or 6-membered heterocyclyl
R⁵ is unsubstituted C₁-C₆ alkyl; or C₁-C₆ alkyl substituted with one or more substituents independently selected from 5-membered heterocyclyl, 6-membered heterocyclyl, 5-membered aryl, 6-membered aryl, 5-membered heteroaryl, or 6-membered heteroaryl; and
R⁶ is unsubstituted cyclopentyl or cyclohexyl; or cyclopentyl or cyclohexyl substituted with one or more NH₂;
wherein when R^{a}, R^{b} and R¹ are each H and R² is H or —COR³, then n is 0 or 1;
or
(ii) a compound of formula (II): 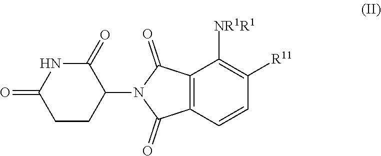
or a pharmaceutically acceptable salt, ester, optically active isomer, racemate, solvate, amino acid conjugate, or prodrug thereof,
wherein:
each R¹ is independently selected from H or C₁-C₄ alkyl;
R¹¹ is OH or OR^{5a}; and
R^{5a} is unsubstituted C₁-C₆ alkyl; or C₁-C₆ alkyl substituted with one or more substituents independently selected from 5-membered heterocyclyl, 6-membered heterocyclyl, 5-membered heteroaryl or 6-membered heteroaryl.

21. A method of treating cancer comprising administering a pharmaceutical composition to a subject in need thereof, wherein the pharmaceutical composition comprises:
(i) a compound of formula (Ia), (Ib) or (Ic): 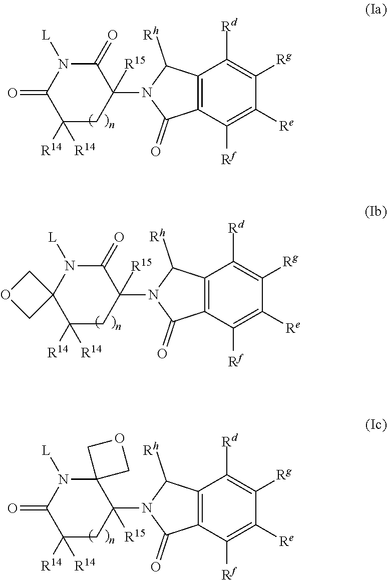
or a pharmaceutically acceptable salt, ester, optically active isomer, racemate, solvate, amino acid conjugate, or prodrug thereof,
wherein
L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, —S(O)₂R″ or —P(O)(OR″)(OR″);
each R″ is independently selected from hydrogen, alkyl, cycloalkyl, alkenyl, cycloalkenyl, aryl, heteroaryl, or benzyl;
each R¹⁴ is independently selected from deuterium or hydrogen;
R¹⁵ is selected from hydrogen, deuterium or C₁-C₄ alkyl;
R^{g} is selected from —COOH or CR^{a}R^{b}R^{c},
R^{h} is selected from H or C₁-C₄ alkyl;
R^{a} is selected from H, deuterium, or C₁-C₄ alkyl;
R^{b} is selected from H, deuterium, or C₁-C₄ alkyl;
R^{c} is selected from NR¹R², OH, OR⁶, CH₂X, CHX₂, or CX₃;
each of R^{d}, R^{e} and R^{f} is independently selected from H, deuterium, X, C₁-C₄ alkyl, or NH₂;
n is 0, 1 or 2;
X is selected from F, Cl, Br or I;
R¹ is selected from H or C₁-C₄ alkyl,
R² is selected from H, C₁-C₄ alkyl, —COR³, or —COOR³,
or R¹ and R², together with the nitrogen atom to which they are attached, form an 5 or 6-membered heterocycle, wherein the heterocycle is unsubstituted or wherein one or more carbon atoms of the heterocycle form part of a carbonyl group; or R¹ and R^{a}, together with the carbon and nitrogen atoms to which they are attached, form a 5 or 6-membered heterocycle;
R³ is selected from:
unsubstituted C₁-C₄ alkyl;
C₁-C₁₀ alkyl substituted with one or more R⁴, wherein each R⁴ is independently selected from NH₂, NHC(NH)NH₂, NHCOR⁵, NHCOOR⁵, OH, OR⁵, OCOR, unsubstituted 5-membered heterocyclyl, substituted or unsubstituted dioxolyl, unsubstituted 6-membered heterocyclyl, 5-membered heteroaryl, 6-membered heteroaryl, indole, or 6-membered aryl substituted with one or more substituents independently selected from C₁-C₄ alkyl, —OH, —CH₂—OH, —OCO(C₁-C₄ alkyl) or —CH₂OCO(C₁-C₄ alkyl); and wherein R⁴ is not X;
C₂-C₁₀ alkyl substituted with a halophenyl group;
6-membered aryl substituted with one or more substituents independently selected from CH₂—OH or CH₂OCO(C₁-C₄ alkyl); and
unsubstituted 5- or 6-membered heterocyclyl;
R⁵ is unsubstituted C₁-C₆ alkyl; or C₁-C₆ alkyl substituted with one or more substituents independently selected from 5-membered heterocyclyl, 6-membered heterocyclyl, 5-membered aryl, 6-membered aryl, 5-membered heteroaryl or 6-membered heteroaryl; and
R⁶ is unsubstituted cyclopentyl or cyclohexyl; or cyclopentyl or cyclohexyl substituted with one or more NH₂;
wherein when R^{a}, R^{b} and R¹ are each H and R² is H or —COR³, then n is 0 or 1;
or
(ii) a compound of formula (II): 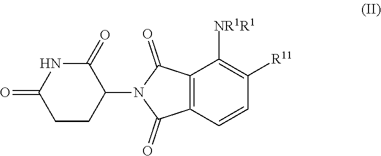
or a pharmaceutically acceptable salt, ester, optically active isomer, racemate, solvate, amino acid conjugate, or prodrug thereof,
wherein:
each R¹ is independently selected from H or C₁-C₄ alkyl;
R¹¹ is OH or OR⁵a; and
R^{5a} is unsubstituted C₁-C₆ alkyl; or C₁-C₆ alkyl substituted with one or more substituents independently selected from 5-membered heterocyclyl, 6-membered heterocyclyl, 5-membered heteroaryl, or 6-membered heteroaryl.

22. The method of claim 20, wherein R³ is selected from:
unsubstituted C₁-C₄ alkyl;
C₁-C₁₀ alkyl substituted with one or more R⁴, wherein each R⁴ is independently selected from —NH₂, —NHC(NH)NH₂, —NHCOR⁵, —NHCOOR⁵, —OH, —OR⁵, —OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, or 6-membered aryl substituted with one or more substituents independently selected from C₁-C₄ alkyl, —OH, —CH₂—OH, —OCO(C₁-C₄ alkyl) or —CH₂OCO(C₁-C₄ alkyl); and wherein R⁴ is not X;
6-membered aryl substituted with one or more substituents independently selected from CH₂—OH or CH₂OCO(C₁-C₄ alkyl); and
unsubstituted 5- or 6-membered heterocyclyl.

23. The method of claim 20, wherein R³ is selected from:
unsubstituted C₁-C₄ alkyl;
C₁-C₁₀ alkyl substituted with one or more R⁴, wherein each R⁴ is independently selected from —NH₂, —NHC(NH)NH₂, —NHCOR⁵, —NHCOOR⁵, —OH, —OR⁵, —OCOR⁵, unsubstituted 5-membered heterocyclyl, substituted or unsubstituted dioxolyl, unsubstituted 6-membered heterocyclyl, 5-membered heteroaryl, 6-membered heteroaryl, indole, or 6-membered aryl substituted with one or more substituents independently selected from C₁-C₄ alkyl, —OH, —CH₂—OH, —OCO(C₁-C₄ alkyl) or —CH₂OCO(C₁-C₄ alkyl); wherein R⁴ is not X, and wherein when the C₁-C₁₀ alkyl is substituted with indole, the C₁-C₁₀ alkyl is further substituted with at least one additional R⁴;
6-membered aryl substituted with one or more substituents independently selected from CH₂—OH or CH₂OCO(C₁-C₄ alkyl); and
unsubstituted 5- or 6-membered heterocyclyl.

24. The method of claim 20, wherein R¹¹ is OH.

25. The method of claim 20, wherein NR¹R¹ is NH₂.

26. The method of claim 20, wherein R^{h} is H

27. The method of claim 20, wherein R^{a} and R^{b} are each H.

28. The method of claim 20, wherein R^{a} and R^{b} are each deuterium.

29. The method of claim 20, wherein R^{a} is H and R^{b} is methyl.

30. The method of claim 20, wherein R^{c} is selected from NHR² or OH.

31. The method of claim 20, wherein the compound is selected from:

| Compound | |
|---|---|
| ID | Structure |
| 1 | 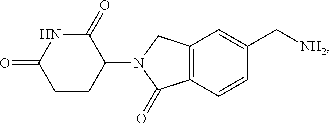 [SMILES: NCc1ccc2c(c1)CN(C1CCC(=O)NC1=O)C2=O] |
| 2 | 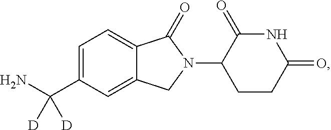 [SMILES: [2H]C([2H])(N)c1ccc2c(c1)CN(C1CCC(=O)NC1=O)C2=O] |
| 3 | 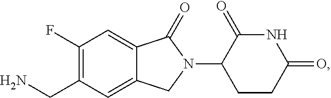 [SMILES: NCc1cc2c(cc1F)C(=O)N(C1CCC(=O)NC1=O)C2] |
| 4 | 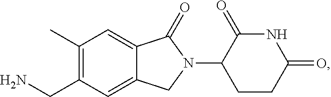 [SMILES: Cc1cc2c(cc1CN)CN(C1CCC(=O)NC1=O)C2=O] |
| 5 | 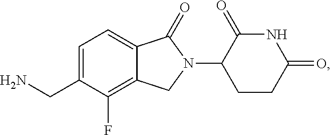 [SMILES: NCc1ccc2c(c1F)CN(C1CCC(=O)NC1=O)C2=O] |
| 6 | 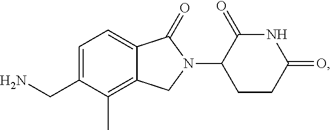 [SMILES: Cc1c(CN)ccc2c1CN(C1CCC(=O)NC1=O)C2=O] |
| 7 | 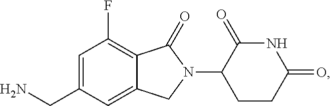 [SMILES: NCc1cc(F)c2c(c1)CN(C1CCC(=O)NC1=O)C2=O] |
| 8 | 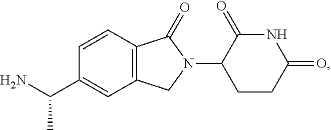 [SMILES: C[C@H](N)c1ccc2c(c1)CN(C1CCC(=O)NC1=O)C2=O] |
| 9 | 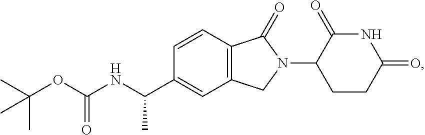 [SMILES: C[C@H](NC(=O)OC(C)(C)C)c1ccc2c(c1)CN(C1CCC(=O)NC1=O)C2=O] |
| 10 | 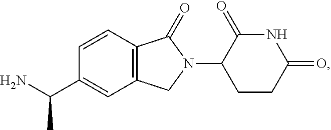 [SMILES: C[C@@H](N)c1ccc2c(c1)CN(C1CCC(=O)NC1=O)C2=O] |
| 11 | 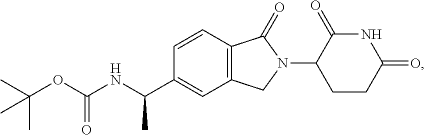 [SMILES: C[C@@H](NC(=O)OC(C)(C)C)c1ccc2c(c1)CN(C1CCC(=O)NC1=O)C2=O] |
| 12 | 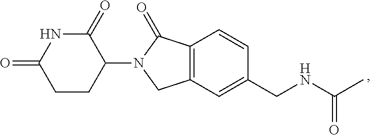 [SMILES: CC(=O)NCc1ccc2c(c1)CN(C1CCC(=O)NC1=O)C2=O] |
| 13 | 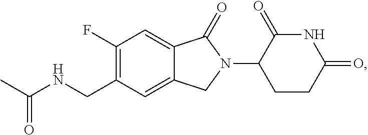 [SMILES: CC(=O)NCc1cc2c(cc1F)C(=O)N(C1CCC(=O)NC1=O)C2] |
| 14 | 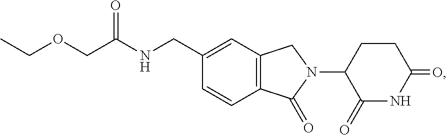 [SMILES: CCOCC(=O)NCc1ccc2c(c1)CN(C1CCC(=O)NC1=O)C2=O] |
| 15 | 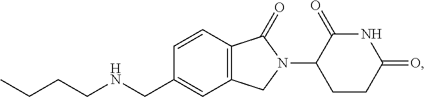 [SMILES: CCCCNCc1ccc2c(c1)CN(C1CCC(=O)NC1=O)C2=O] |
| 16 | 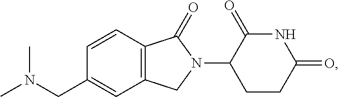 [SMILES: CN(C)Cc1ccc2c(c1)CN(C1CCC(=O)NC1=O)C2=O] |
| 17 | 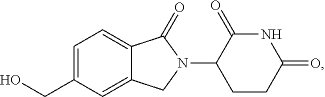 [SMILES: O=C1CCC(N2Cc3cc(CO)ccc3C2=O)C(=O)N1] |
| 18 | 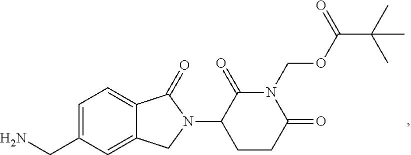 [SMILES: CC(C)(C)C(=O)OCN1C(=O)CCC(N2Cc3cc(CN)ccc3C2=O)C1=O] |
| 19 | 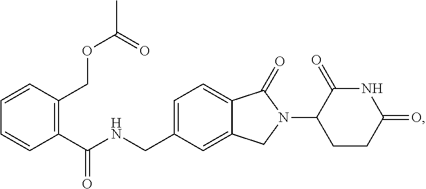 [SMILES: CC(=O)OCc1ccccc1C(=O)NCc1ccc2c(c1)CN(C1CCC(=O)NC1=O)C2=O] |
| 20 | 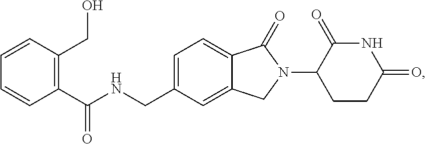 [SMILES: O=C1CCC(N2Cc3cc(CNC(=O)c4ccccc4CO)ccc3C2=O)C(=O)N1] |
| 21 | 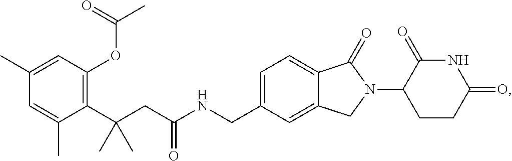 [SMILES: CC(=O)Oc1cc(C)cc(C)c1C(C)(C)CC(=O)NCc1ccc2c(c1)CN(C1CCC(=O)NC1=O)C2=O] |
| 22 | 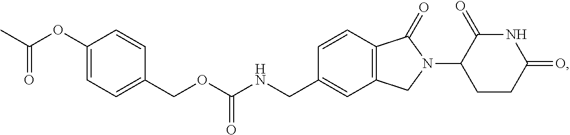 [SMILES: CC(=O)Oc1ccc(COC(=O)NCc2ccc3c(c2)CN(C2CCC(=O)NC2=O)C3=O)cc1] |
| 23 | 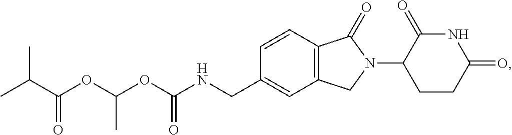 [SMILES: CC(OC(=O)NCc1ccc2c(c1)CN(C1CCC(=O)NC1=O)C2=O)OC(=O)C(C)C] |
| 24 | 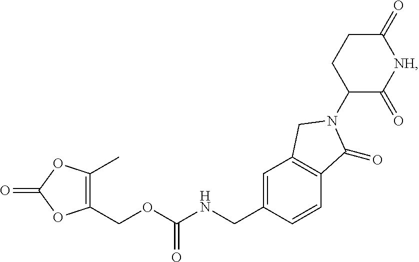 [SMILES: Cc1oc(=O)oc1COC(=O)NCc1ccc2c(c1)CN(C1CCC(=O)NC1=O)C2=O] |
| 25 | 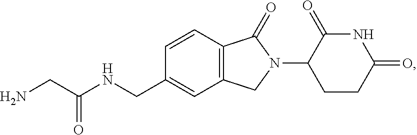 [SMILES: NCC(=O)NCc1ccc2c(c1)CN(C1CCC(=O)NC1=O)C2=O] |
| 26 | 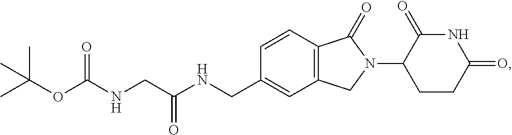 [SMILES: CC(C)(C)OC(=O)NCC(=O)NCc1ccc2c(c1)CN(C1CCC(=O)NC1=O)C2=O] |
| 27 | 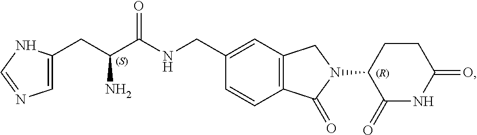 |
| 28 | 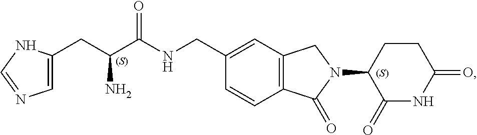 |
| 29 | 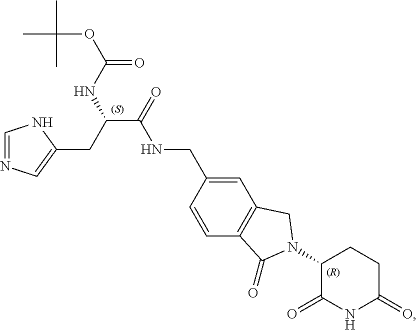 |
| 30 | 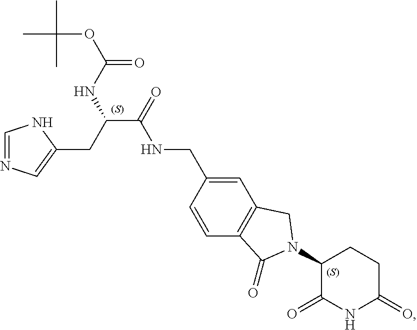 |
| 31 | 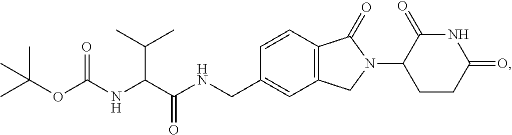 [SMILES: CC(C)C(NC(=O)OC(C)(C)C)C(=O)NCc1ccc2c(c1)CN(C1CCC(=O)NC1=O)C2=O] |
| 32 | 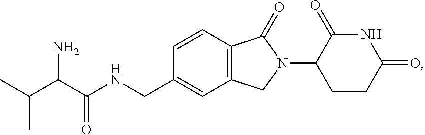 [SMILES: CC(C)C(N)C(=O)NCc1ccc2c(c1)CN(C1CCC(=O)NC1=O)C2=O] |
| 33 | 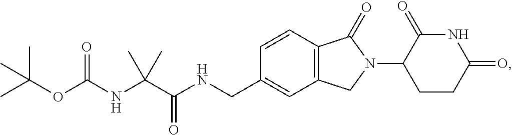 [SMILES: CC(C)(C)OC(=O)NC(C)(C)C(=O)NCc1ccc2c(c1)CN(C1CCC(=O)NC1=O)C2=O] |
| 34 | 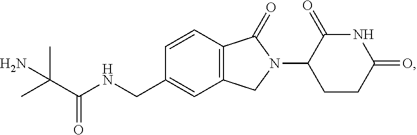 [SMILES: CC(C)(N)C(=O)NCc1ccc2c(c1)CN(C1CCC(=O)NC1=O)C2=O] |
| 35 | 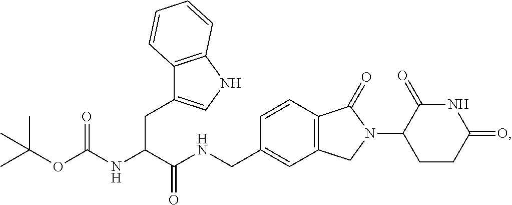 [SMILES: CC(C)(C)OC(=O)NC(Cc1c[nH]c2ccccc12)C(=O)NCc1ccc2c(c1)CN(C1CCC(=O)NC1=O)C2=O] |
| 36 | 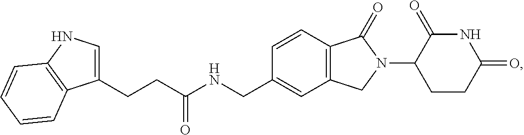 [SMILES: O=C(CCc1c[nH]c2ccccc12)NCc1ccc2c(c1)CN(C1CCC(=O)NC1=O)C2=O] |
| 37 | 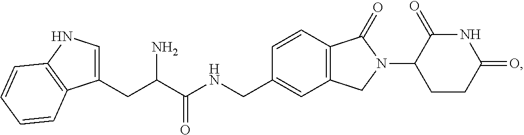 [SMILES: NC(Cc1c[nH]c2ccccc12)C(=O)NCc1ccc2c(c1)CN(C1CCC(=O)NC1=O)C2=O] |
| 38 | 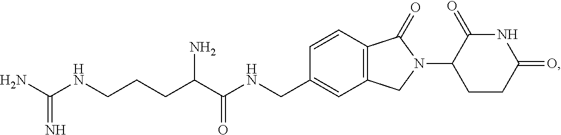 [SMILES: N=C(N)NCCCC(N)C(=O)NCc1ccc2c(c1)CN(C1CCC(=O)NC1=O)C2=O] |
| 39 | 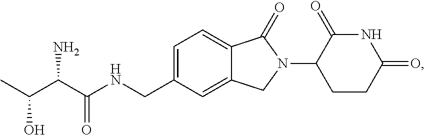 [SMILES: C[C@@H](O)[C@H](N)C(=O)NCc1ccc2c(c1)CN(C1CCC(=O)NC1=O)C2=O] |
| 40 | 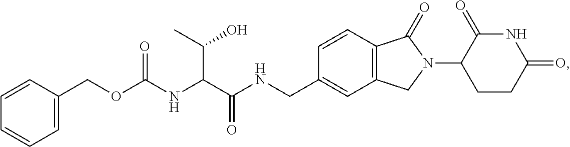 [SMILES: C[C@H](O)C(NC(=O)OCc1ccccc1)C(=O)NCc1ccc2c(c1)CN(C1CCC(=O)NC1=O)C2=O] |
| 41 | 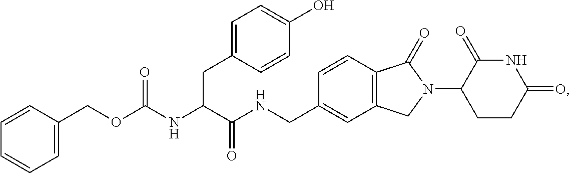 [SMILES: O=C1CCC(N2Cc3cc(CNC(=O)C(Cc4ccc(O)cc4)NC(=O)OCc4ccccc4)ccc3C2=O)C(=O)N1] |
| 42 | 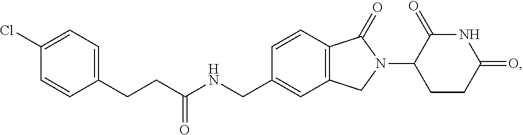 [SMILES: O=C(CCc1ccc(Cl)cc1)NCc1ccc2c(c1)CN(C1CCC(=O)NC1=O)C2=O] |
| 43 | 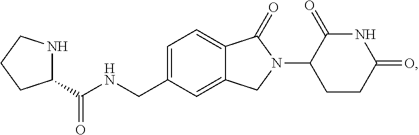 [SMILES: O=C1CCC(N2Cc3cc(CNC(=O)[C@@H]4CCCN4)ccc3C2=O)C(=O)N1] |
| 44 | 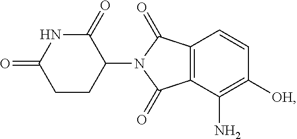 [SMILES: Nc1c(O)ccc2c1C(=O)N(C1CCC(=O)NC1=O)C2=O] |
| 50 | 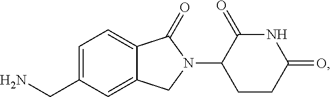 [SMILES: NCc1ccc2c(c1)CN(C1CCC(=O)NC1=O)C2=O] |
| 51 | 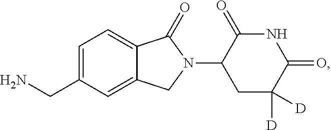 [SMILES: [2H]C1([2H])CC(N2Cc3cc(CN)ccc3C2=O)C(=O)NC1=O] |
| 52 | 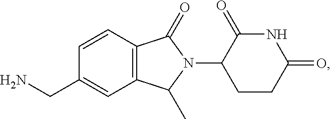 [SMILES: CC1c2cc(CN)ccc2C(=O)N1C1CCC(=O)NC1=O] |
| 53 | 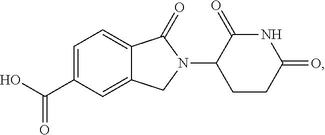 [SMILES: O=C1CCC(N2Cc3cc(C(=O)O)ccc3C2=O)C(=O)N1] |
| 54 | 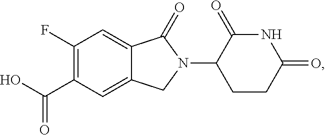 [SMILES: O=C1CCC(N2Cc3cc(C(=O)O)c(F)cc3C2=O)C(=O)N1] |
| 48 | 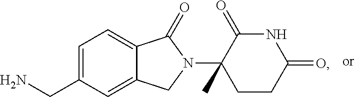 [SMILES: C[C@]1(N2Cc3cc(CN)ccc3C2=O)CCC(=O)NC1=O] |
| 49 | 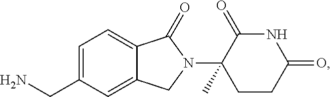 [SMILES: C[C@@]1(N2Cc3cc(CN)ccc3C2=O)CCC(=O)NC1=O] |

or a pharmaceutically acceptable salt, ester, optically active isomer, racemate, solvate, amino acid conjugate, or prodrug thereof.

32. The method of claim 20, wherein R^{c} is NHR².

33. The method of claim 20, wherein R² is selected from H, —COR³, or —COOR³.

34. The method of claim 20, wherein R³ is C₁-C₁₀ alkyl substituted with one or more R⁴.

35. The method of claim 20, wherein each R⁴ is independently selected from NH₂, OCOR⁵, indole or 6-membered aryl substituted with one or more —OCO(C₁-C₄ alkyl); wherein R⁴ is not X.

36. The method of claim 20, wherein the compound is selected from:

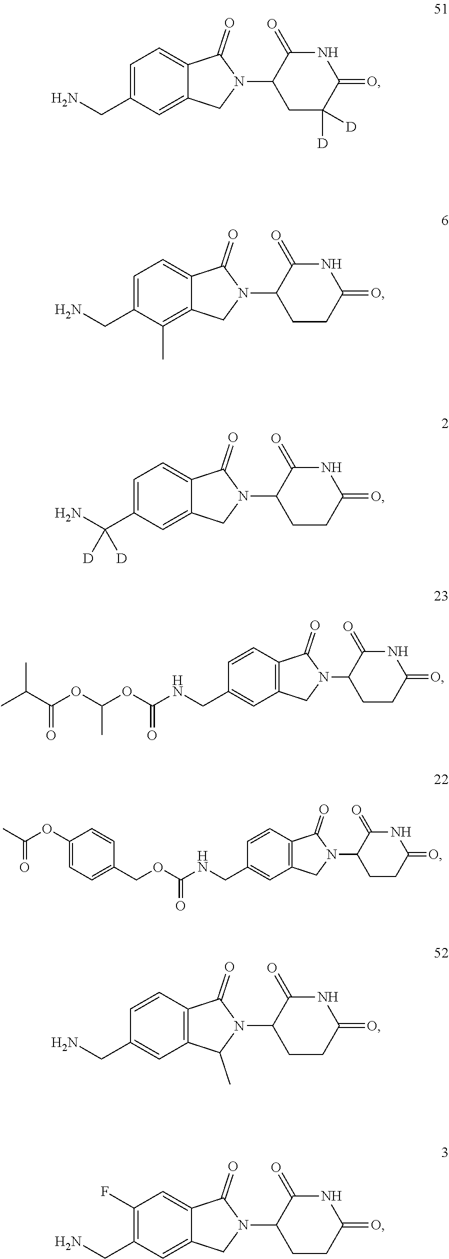

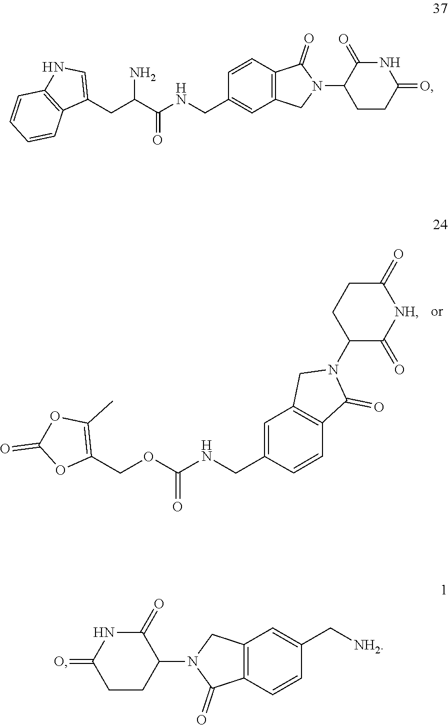 [SMILES: NC(Cc1c[nH]c2ccccc12)C(=O)NCc1ccc2c(c1)CN(C1CCC(=O)NC1=O)C2=O ; Cc1oc(=O)oc1COC(=O)NCc1ccc2c(c1)CN(C1CCC(=O)NC1=O)C2=O ; NCc1ccc2c(c1)CN(C1CCC(=O)NC1=O)C2=O]

37. The method of claim 20, wherein the cancer is associated with SALL4, GSPT1, or a combination thereof.

38. The method of claim 20, wherein the cancer is hepatocellular carcinoma, neuroblastoma, leukemia, acute myeloid leukemia (AML), acute promyelocytic leukemia (APL), multiple myeloma, breast cancer, prostate cancer, bladder cancer, kidney cancer, muscle cancer, ovary cancer, skin cancer, pancreas cancer, breast cancer, colon cancer, hematological cancer, cancer of a connective tissue, placenta cancer, bone cancer, uterus cancer, cervical cancer, choriocarcinoma, endometrial cancer, gastric cancer, or lung cancer.

39. The method of claim 38, wherein the cancer is hepatocellular carcinoma, neuroblastoma, leukemia, prostate cancer, or multiple myeloma.

40. The method of claim 38, wherein the cancer is hepatocellular carcinoma.

41. The method of claim 31, wherein the compound is:
(a) selected from compounds 6, 3, 36, 42, 26, 23, 24, 1, 52, 28, 27, 37, 39, 38, or 5; or
(b) selected from compounds 6, 3, 36, 42, 26, 23, 24, 1, or 52.

42. The method of claim 38, wherein the cancer is neuroblastoma.

43. The method of claim 42, wherein the compound is

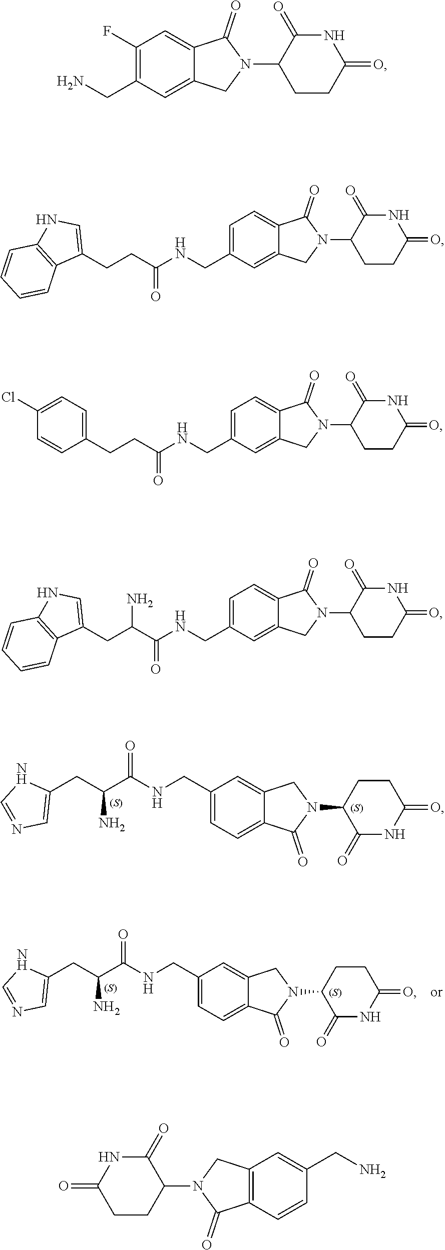

44. The method of claim 38, wherein the cancer is leukemia.

45. The method of claim 44, wherein the compound is

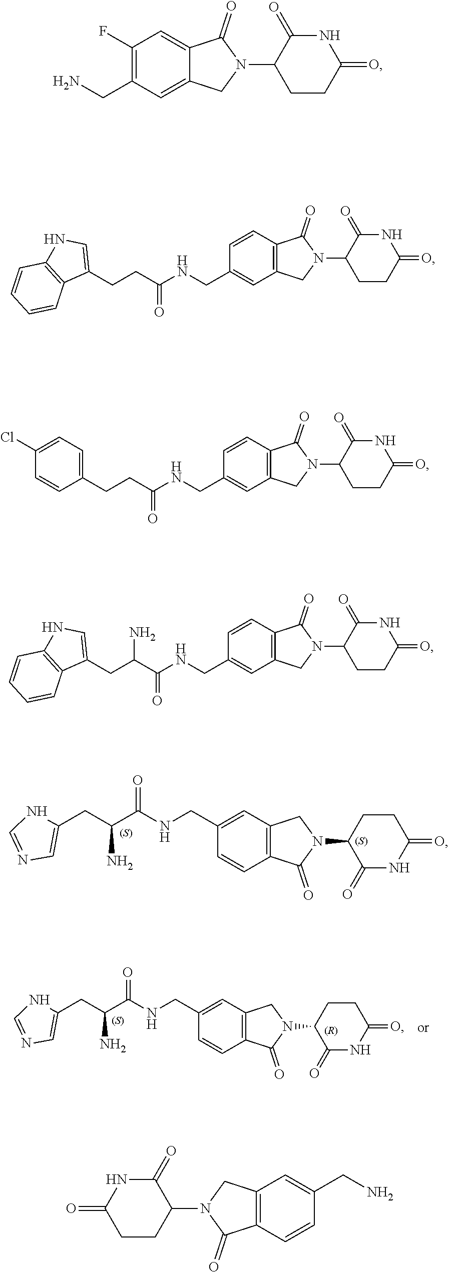

46. The method of claim 20, wherein the method further comprises administering a second cancer therapy to the subject.

47. The method of claim 46, wherein the second cancer therapy is chemotherapy, radiotherapy or immunotherapy.

48. The method of claim 46, wherein the second agent is selected from a therapeutic antibody that specifically binds to a cancer antigen, a hematopoietic growth factor, a cytokine, anti-cancer agent, an antibiotic, a cox-2 inhibitor, an immunomodulatory agent, an immunosuppressive agent, a corticosteroid or a pharmacologically active mutant or derivative thereof.

49. The compound of claim 20, wherein the method comprises oral administration of the compound or the pharmaceutical composition to the subject.

50. The compound of claim 1, wherein the compound is of formula (Ia).

51. The compound of claim 1, wherein the compound is of formula (Ib).

52. The compound of claim 1, wherein the compound is of formula (Ic).

53. The compound of claim 1, wherein the compound is of formula (Ia) or formula (Ic).

54. The compound of claim 2, wherein the compound is of formula (II).

55. The compound of claim 1, wherein L is selected from hydrogen, alkyl, alkenyl, benzyl, aryl, heteroaryl, haloalkyl, haloalkenyl, —CH₂OC(O)^{t}Bu, —CH₂C(O)OR″, —C(O)R″, —C(O)OR″, —C(O)NH₂, —C(O)NHR″, —C(O)NR″₂, —OR″, —NR″₂, or —S(O)₂R″.

56. The compound of claim 55, wherein L is alkyl, benzyl, —CH₂OC(O)Me, or —CH₂OC(O)^{t}Bu-.

57. The compound of claim 55, wherein L is hydrogen.

58. The compound of claim 1, wherein n is 1.

59. The compound of claim 1, wherein n is 0.

60. The compound of claim 1, wherein each R¹⁴ is deuterium.

61. The compound of claim 1, wherein each R¹⁴ is hydrogen.

62. The compound of claim 1, wherein R¹⁵ is deuterium.

63. The compound of claim 1, wherein R¹⁵ is hydrogen.

64. The compound of claim 1, wherein R^{e} is X.

65. The compound of claim 1, wherein R¹ is selected from H or methyl.

66. The compound of claim 1, wherein R² is selected from H, methyl, —COR³, or —COOR³.

67. The method of claim 20, wherein administration of the compound or pharmaceutical composition to a subject reduces levels of a target protein in the subject.

68. The method of claim 67, wherein the target protein is selected from SALL-4 or GSPT1.

69. The method of claim 67, wherein administration of the compound or pharmaceutical composition to the subject induces minimal reduction or substantially no reduction of IKZF1 or IKZF3 protein levels.
